(19) United States
(12) Patent Application Publication
(10) Pub. No.: US 2005/0004163 A1
(43) Pub. Date: Jan. 6, 2005

(54) 3-(diarylmethylene)-8-azabicyclo[3.2.1]octane derivatives

(76) Inventors: Steven J. Coats, Quakertown, PA (US); John R. Carson, Norristown, PA (US); Lou Anne Neilson, Sellersville, PA (US); Philip M. Pitis, North Wales, PA (US); Mark J. Schulz, Skippack, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/767,712

(22) Filed: Jan. 29, 2004

Related U.S. Application Data
(63) Continuation-in-part of application No. 10/360,859, filed on Feb. 7, 2003, now abandoned.
Application No. 10/360,859 is a continuation of application No. 09/791,246, filed on Feb. 22, 2001, now Pat. No. 6,552,036.
(60) Provisional application No. 60/186,778, filed on Mar. 3, 2000.

(51) Int. Cl. C07D 451/02; A61K 31/46

(52) U.S. Cl. 514/304; 546/124; 546/125

(57) ABSTRACT

This invention is directed to 3-(diarylmethylene)-8-azabicyclo[3.2.1]octane derivatives useful as δ-opioid or μ-opioid receptor modulators. Depending on their agonist or antagonist effect, the compounds are useful analgesics, immunosuppressants, antiinflammatory agents, agents for the treatment of neurological and psychiatric conditions, medicaments for drug and alcohol abuse, agents for treating gastritis and diarrhea, cardiovascular agents and agents for the treatment of respiratory diseases.

### CROSS REFERENCE TO RELATED APPLICATIONS

This application is a continuation in part of U.S. patent application Ser. No. 10/360,859 filed on Feb. 7, 2003, which application is fully incorporated herein by reference, and which applicaiton is a continuation of U.S. patent application Ser. No. 09/791,246, filed Feb. 22, 2001 which application is fully incorporated herein by reference, and which application claims the benefit of U.S. Provisional Application No. 60/186,778 filed Mar. 3, 2000, also fully incorporatated herein by reference.

### FIELD OF THE INVENTION

The present invention is directed to compounds useful as delta-opioid and mu-opioid receptor modulators. More particularly, the present invention is directed to 3-(diarylmethylene)-8-azabicyclo[3.2.1]octane derivatives useful as delta-opioid or mu-opioid receptor modulators.

### BACKGROUND OF THE INVENTION

WO 97/23466 describes compounds as having an analgesic effect of a general and one preferred formula:

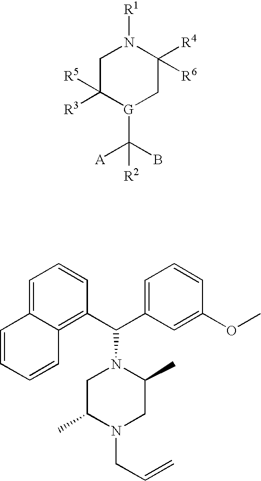

WO 98/28270 describes compounds as having an analgesic effect of a general and one preferred formula:

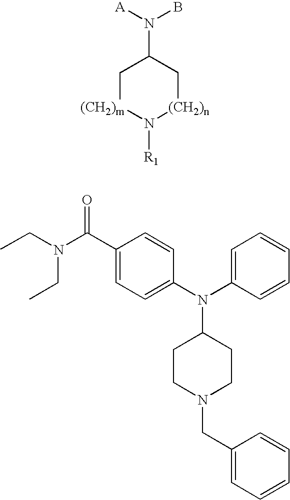

WO 98/28275 describes compounds as having an analgesic effect of a general and one preferred formula:

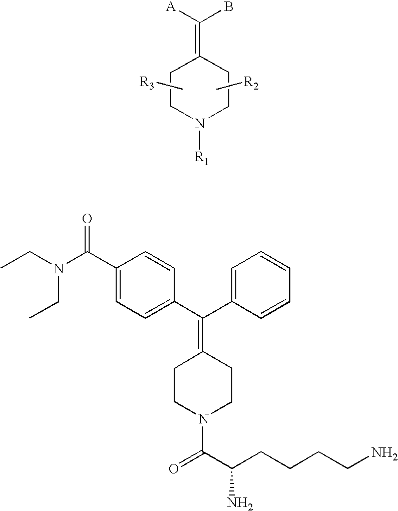

Amide derivatives of 3-aminotropane have been prepared and described as having potential pharmacological activity (Gutkowska, B., et al., Acta Pol. Pharm., 1984, 41(6), 613-617), of the formula:

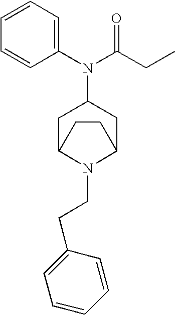

WO 93/15062 describes compounds as delta-opioid (δ-opioid) and mu-opioid (μ-opioid) receptor agonists of (approximately) the general formula:

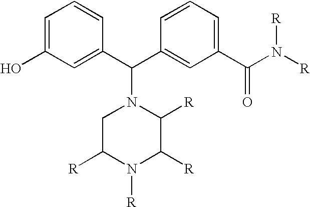

The synthesis and binding affinities for 4-Diarylaminotropane compounds as δ-opioid agonists have been described (Boyd, R. E., Carson, J. R., Codd, E. E., Gauthier, A. D., Neilson, L. A and Zhang, S-P., Biorg. Med. Chem. Lett., 2000,10: 1109-1111) of the general formula: 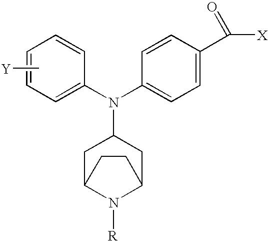
wherein R is hydrogen, methyl, propyl, hexyl, 2-ethylbutyl, allyl, 3,3-dimethallyl, cyclohexylmethyl, phenethyl, phenylpropyl, 2,2-diphenylethyl, 3,4-dimethoxyphenethyl, 4-fluorophenethyl, 2-furylmethyl, 3,4-methylenedioxybenzyl, cyano and X is N,N-dimethylamino, N,N-diethylamino, N,N-dipropylamino, N-methyl-N-ethylamino, N-methyl-N-propylamino, N-methyl-N-phenylamino, N-ethyl-N-(4-methyl)benzylamino, N-butyl-N-ethylamino, N-butyl-N-propylamino, [N-ethyl-N-(2-methyl)allyl]amino, hydroxy, O-t-butyl and 1-pyrrolidinyl; and, Y is hydrogen, methoxy and methylthio.

Other selective 4-[(8-alkyl-8-azabicyclo[3.2.1]octyl-3-yl)-3-arylanilino]-N,N-diethylbenzamide δ-opioid ligands have also been described (Thomas, J. B., Atkinson, R. N., Rothman, R. B., Burgess, J. P., Mascarella, S. W., Dersch, C. M., Xu, H. and Carroll, F. I., Biorg. Med. Chem. Lett., 2000,10: 1281-1284).

The present invention is directed to compounds useful as delta-opioid and mu-opioid receptor modulators. More particularly, the present invention is directed to delta-opioid and mu-opioid receptor modulators.

Thus the present invention to provides 3-(diarylmethylene)-8-azabicyclo[3.2.1]octane derivatives useful as δ-opioid or 1′-opioid receptor modulators. The present invention also provides δ-opioid and μ-opioid receptor selective agonists as analgesics having reduced side-effects. The present invention also provides δ-opioid and μ-opioid receptor selective antagonists as immunosuppressants, antiinflammatory agents, agents for the treatment of neurological and psychiatric conditions, medicaments for drug and alcohol abuse, agents for treating gastritis and diarrhea, cardiovascular agents and agents for the treatment of respiratory diseases, having reduced side-effects. The present invention also provides a useful pharmaceutical composition comprising a compound of the present invention useful as a δ-opioid or μ-opioid receptor modulator. The present invention also provides a useful pharmaceutical composition comprising a δ-opioid or μ-opioid receptor modulator compound of Formula (I) in combination with a μ-opioid receptor modulator or a δ-opioid or μ-opioid receptor modulator compound of Formula (I) wherein the combination has a synergistic therapeutic effect.

### SUMMARY OF THE INVENTION

The present invention provides an opioid receptor modulator compound selected from the group consisting of a δ-opioid and a μ-opioid receptor modulator compound of Formula (Ia): 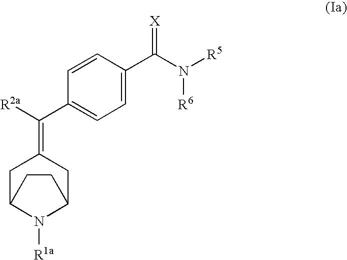
wherein:
R^{1a} is a substituent selected from the group consisting of hydrogen, C₁₋₆alkyl, —CH₂—(C₂₋₈alkenyl), cycloalkyl(C₁₋₄)alkyl, heterocyclyl(C₁₋₈)alkyl, aryl(C₁₋₈)alkyl, aryl(C₂₋₈)alkynyl, heteroaryl(C₁₋₈)alkyl, (R¹¹)₂—N—(C₁₋₈)alkyl, R¹¹—O—(C₁₋₈)alkyl-, R¹¹—S—(C₁₋₈)alkyl, R¹¹—SO—(C₁₋₈)alkyl, and R¹¹—SO₂—(C₁₋₈)alkyl; wherein heterocyclyl is optionally substituted with one to three substituents independently selected from the group consisting of C₁₋₆alkyl, C₁₋₆alkoxy, C₁₋₆alkoxycarbonyl, C₁₋₆alkylcarbonylamino, C₁₋₆alkylthio, C₁₋₆alkylsulfonyl, halogen, and oxo; and wherein aryl and heteroaryl are optionally substituted with one to three substituents independently selected from the group consisting of C₁₋₆alkyl, C₂₋₆alkenyl, C₁₋₆alkoxy, amino, C₁₋₆alkylamino, di(C₁₋₆alkyl)amino, C₁₋₆alkylcarbonyl, C₁₋₆alkoxycarbonylamino, C₁₋₆alkylthio, C₁₋₆alkylsulfonyl, heterocyclyl, cyano, halogen, hydroxy, trifluoromethyl and trifluoromethoxy; wherein R¹¹ is hydrogen, C₁₋₈alkyl or aryl;
R^{2a} is a substituent selected from hydrogen, halogen, cyano, [1,3]-benzodioxolyl, quinolinyl, tetrazolyl, or aryl; wherein aryl is substituted with one to three substituents independently selected from the group consisting of C₁₋₆alkyl, carboxy, amino and carboxy, nitro, di(C₁₋₆alkyl)aminocarbonyl, (C₁₋₆alkyl)aminocarbonyl, aminocarbonyl, aminosulfonyl, or tetrazolyl; and wherein alkyl is substituted with one to three substituents selected from amino, hydroxy, or carboxy;
X is selected from O or S.
R⁵ and R are independently selected from hydrogen or C₁₋₈alkyl;
   The present invention is directed to compounds having Formula (Ib): 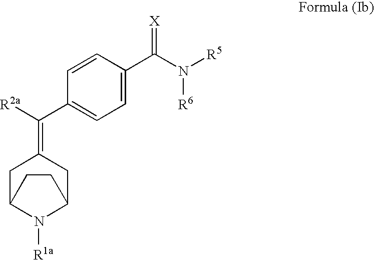
      wherein:
R^{1b} is a substituent selected from the group consisting of (1-benzyl-1-amino)ethyl, 1-benzyl-1-(t-butoxycarbonylamino)ethyl, 2-(4-alkoxycarbonylpiperazin-1-yl)eth-1-yl, 3-dimethylaminocarbonyl-3,3-diphenylprop-1-yl, 3-cyano-3,3-diphenylprop-1-yl, tetrazolyl(C₁₋₃)alkyl, quinolinyl(C₁₋₃)alkyl, aryl(C₁₋₄)alkyl, aryl(C₁₋₄)alkylcarbonyl, heteroarylcarbonyl, (halo-arylcarbonyl)heteroarylcarbonyl(C₁₋₃)alkyl, (C₁₋₄)alkoxycarbonyl, cyano, cyano(C₁₋₃)alkyl, formyl, and aminoiminomethyl; wherein aryl and heteroaryl are substituted with one to three substituents independently selected from the group consisting of C₁₋₆alkylcarbonylamino, carboxy, and nitro;
R^{2b} is a substituent selected from aryl or heteroaryl; wherein aryl and monocyclic heteroaryl are optionally substituted with C₁₋₆alkyl, C₁₋₆alkoxy, amino, C₁₋₆alkylamino, di(C₁₋₆alkyl)amino, C₁₋₆alkylcarbonyl, C₁₋₆alkylcarbonylamino, C₁₋₆alkylthio, C₁₋₆alkylsulfonylamino, halogen, hydroxy, cyano, trifluoromethyl and trifluoromethoxy;
X, R⁵, and R⁶ are as described above;
   The present invention is also directed to compounds having Formula (Ic) 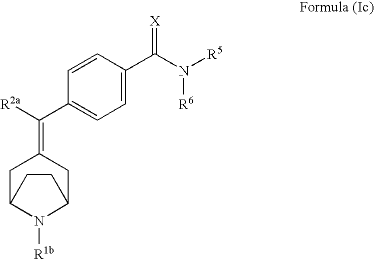
      wherein:
R^{1b}, R^{2a}, X, R⁵, and R⁶ are as previously defined.
and pharmaceutically acceptable enantiomers, diastereomers and salts thereof.

### DETAILED DESCRIPTION OF THE INVENTION

Embodiments include compounds of Formulas (Ia) and (Ic) wherein, preferably, R^{1a} is selected from the group consisting of hydrogen, —CH₂—C₂₋₆alkenyl, heterocyclyl(C₁₋₃)alkyl, heteroaryl(C₁₋₃)alkyl, aryl(C₁₋₃)alkyl, aryl(C₂₋₃)alkynyl; and wherein aryl and heteroaryl are independently and optionally substituted with one to three substituents independently selected from the group consisting of C₁₋₆alkyl, C₁₋₆alkoxy, C₁₋₆alkylcarbonylamino, halogen, hydroxy, C₁₋₆alkylcarbonyl, and cyano.

More preferably for compounds of Formulas (Ia) and (Ic), R^{1a} is selected from the group consisting of hydrogen, 3,3-dimethallyl, (1,3)-benzodioxol-5-yl(C₁₋₃)alkyl, phenyl(C₁₋₃)alkyl, phenyl(C₂₋₃)alkynyl, imidazolinyl(C₁₋₃)alkyl, furyl(C₁₋₃)alkyl, thiophenyl(C₁₋₃)alkyl, thiazolyl(C₁₋₃)alkyl, imidazolyl(C₁₋₃)alkyl, and pyridinyl(C₁₋₃)alkyl; wherein thiophenyl, furyl, imidazolyl, and phenyl are optionally substituted with one to three substituents selected from halogen, C₁₋₃alkylcarbonylamino, and C₁₋₃alkyl.

Embodiments include compounds of Formulas (Ia) and (Ic) wherein, preferably, R¹¹ is independently selected from the group consisting of hydrogen, C₁₋₈alkyl and aryl. More preferably, R¹¹ is independently selected from the group consisting of hydrogen, methyl, and phenyl.

Most preferably for compounds of formulas (Ia) and (Ic), R^{1a} is selected from the group consisting of hydrogen, 3,3-dimethallyl, phenethyl, phenylpropyl, imidazolylmethyl, thiophenylmethyl, (1,3)-benzodioxol-5-ylmethyl, pyridinylmethyl, thiazolylmethyl, and furylmethyl; wherein phenyl and thiophenyl are optionally substituted with one to two substituents selected from halogen, acetamido, or methyl.

Embodiments include compounds of Formulas (Ia) and (Ic) wherein, preferably, R^{2a} is selected from the group consisting of hydrogen, halogen, cyano, phenyl, tetrazolyl, 1,3-benzodioxolyl, and quinolinyl; wherein phenyl is substituted with one to three substituents independently selected from the group consisting of C₁₋₃alkyl, amino (when said phenyl is also substituted with carboxy), aminocarbonyl, C₁₋₆alkylaminocarbonyl, di(C₁₋₆alkyl)aminocarbonyl, aminosulfonyl, heteroaryl, nitro, and carboxy; wherein alkyl is substituted with one to three substituents independently selected from amino, C₁₋₆alkylamino, di(C₁₋₆alkyl)amino, hydroxy, or carboxy.

More preferably for compounds of formulas (Ia) and (Ic), R^{2a} is selected from the group consisting of hydrogen, halogen, cyano, phenyl, tetrazolyl, and (1,3)-benzodioxolyl; wherein phenyl is optionally substituted with one to three substituents independently selected from the group consisting of C₁₋₄alkyl, aminocarbonyl, alkylaminocarbonyl, di(C₁₋₆alkyl)aminocarbonyl, aminosulfonyl, heteroaryl, nitro, carboxy, and cyano; wherein tetrazolyl is optionally substituted with C₁₋₃alkyl; and wherein alkyl is substituted with one to three substituents independently selected from amino, hydroxy, and carboxy.

Most preferably for compounds of formulas (Ia) and (Ic), R^{2a} is selected from the group consisting of hydrogen, bromine, cyano, phenyl, tetrazolyl, and (1,3)-benzodioxolyl; wherein phenyl is optionally substituted with one to three substituents independently selected from the group consisting of aminocarbonyl, ethylaminocarbonyl, dimethylaminocarbonyl, hydroxymethyl, carboxyethyl, carboxy(1-amino)ethyl, aminosulfonyl, tetrazolyl, nitro, and carboxy.

Embodiments also include compounds of Formulas (Ib) and (Ic) wherein, preferably, R^{1b} is selected from the group consisting of aryl(C₁₋₄)alkylcarbonyl, heteroaryl(C₁₋₄)alkyl, heteroarylcarbonyl, cyano(C₁₋₄)alkyl, quinolinyl(C₁₋₃)alkyl, (3-dimethylaminocarbonyl-3,3-diphenylprop-1-yl, (1-benzyl-1-amino)ethyl, 2-(4-alkoxycarbonylpiperazin-1-yl)eth-1-yl, 3-cyano-3,3-diphenylprop-1-yl, (halo-arylcarbonyl)heteroarylcarbonyl(C₁₋₃)alkyl, tetrazolyl(C₁₋₃)alkyl, (C₁₋₄)alkoxycarbonyl, and aminoiminomethyl; wherein heteroaryl is substituted with one to three substituents independently selected from carboxy, halogen, or nitro.

More preferably for compounds of formulas (Ib) and (Ic), R^{1b} is selected from the group consisting of quinolinyl(C₁₋₃)alkyl, aminoiminomethyl, aryl(C₁₋₄)alkylcarbonyl, and heteroaryl(C₁₋₄)alkyl wherein heteroaryl is substituted with nitro.

Most preferably for compounds of formulas (Ib) and (Ic), R^{1b} is selected from thiophenylcarbonyl, 5-nitro-thiophen-3-yl, quinolin-2-ylmethyl, benzylcarbonyl, or aminoiminomethyl.

Embodiments include compounds of Formulas (Ib) and (Ic) wherein, preferably, R^{2b} is selected from aryl or heteroaryl; wherein aryl and heteroaryl are optionally substituted with C₁₋₆alkyl, amino, C₁₋₆alkylcarbonylamino, halogen, and cyano.

More preferably for compounds of formulas (Ib) and (Ic), R^{2b} is selected from aryl, pyridinyl, pyrimidinyl, or pyrazinyl; wherein aryl is optionally substituted with amino, C₁₋₆alkylcarbonyl, C₁₋₆alkylcarbonylamino, halogen, or cyano.

Most preferably for compounds of formulas (Ib) and (Ic), R^{2b} is selected from phenyl or pyridinyl; wherein phenyl is optionally substituted with a substituent selected from amino, methylcarbonyl, methylcarbonylamino, fluorine, or cyano.

Embodiments include compounds of formulas (Ia), (Ib), and (Ic) wherein, preferably, X is 0.

Embodiments include compounds of Formulas (Ia), (Ib), and (Ic) wherein, preferably, R⁵ and R⁶ are independently selected from the group consisting of hydrogen and C₁₋₄alkyl.

More preferably for compounds of formulas (Ia), (Ib), and (Ic), R⁵ and R⁶ are independently selected from the group consisting of hydrogen, methyl, and ethyl and pharmaceutically acceptable enantiomers, diastereomers, and salts thereof.

Table 1 lists compounds exemplified in the present invention:

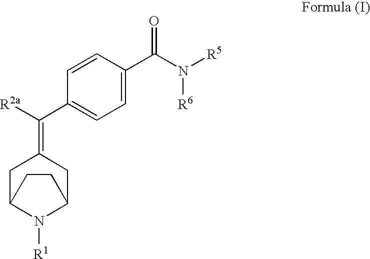

wherein R¹, R⁵ and R⁶ for any compound are delineated in individual rows of Table 1

TABLE 1
(S*, R*) or (R*, S*): enantiomer, unknown absolute

| Cpd | R¹ | R² | R⁵ | R⁶ |
|---|---|---|---|---|
| 1 | 1-benzyl- | H | H | Et |
| | 1-amino-ethyl | | | |
| 2 | 1-benzyl 1-(t- | H | H | Et |
| | butoxycarbonylamino)- | | | |
| | ethyl | | | |
| 3 | quinolin-2-ylmethyl | (3-F)phenyl | H | Et |
| 4 | quinolin-2-ylmethyl | (4-F)phenyl | H | Et |
| 5 | (4-N-acetamido) | benzo[1,3]dioxol-5-yl | H | Et |
| | phenylmethyl | | | |
| 6 | 1H-imidazol-2-ylmethyl | benzo[1,3]dioxol-5-yl | H | Et |
| 7 | thiophen-3ylmethyl | benzo[1,3]dioxol-5-yl | H | Et |
| 8 | furan-2-ylmethyl | benzo[1,3]dioxol-5-yl | H | Et |
| 9 | quinolin-2-ylmethyl | benzo[1,3]dioxol-5-yl | H | Et |
| 10 | furan-3-ylmethyl | benzo[1,3]dioxol-5-yl | H | Et |
| 11 | (5-Me)-3H-imidazol-4- | benzo[1,3]dioxol-5-yl | H | Et |
| | ylmethyl | | | |
| 12 | (3-Me)-thiophen-2-yl | benzo[1,3]dioxol-5-yl | H | Et |
| 13 | quinolin-2-ylmethyl | pyridin-2-yl | H | Et |
| 14 | (4-N-acetamido) | quinolin-3-yl | H | Et |
| | phenylmethyl | | | |
| 15 | thiophen-3-ylmethyl | quinolin-3-yl | H | Et |
| 16 | furan-2-ylmethyl | quinolin-3-yl | H | Et |
| 17 | furan-3-ylmethyl | quinolin-3-yl | H | Et |
| 18 | (3-Me)-thiophen-2-yl | quinolin-3-yl | H | Et |
| 19 | quinolin-2-ylmethyl | (3-amino)phenyl | H | Et |
| 20 | quinolin-2-ylmethyl | (3-CN)phenyl | H | Et |
| 21 | (4-N-acetamido) | Br | H | Et |
| | phenylmethyl | | | |
| 22 | 1H-imidazol-2-ylmethyl | Br | H | Et |
| 23 | thiophen-3-ylmethyl | Br | H | Et |
| 24 | furan-2-ylmethyl | Br | H | Et |
| 25 | quinolin-2-ylmethyl | Br | H | Et |
| 26 | furan-3-ylmethyl | Br | H | Et |
| 27 | (5-Me)-3H-imidazol-4- | Br | H | Et |
| | ylmethyl | | | |
| 28 | (3-Me)-thiophen-2-yl | Br | H | Et |
| 29 | quinolin-2-ylmethyl | (2,6-dimethyl)phenyl | H | Et |
| 30 | quinolin-2-ylmethyl | pyrazin-2-yl | H | Et |
| 31 | (4-N-acetamido) | H | H | Et |
| | phenylmethyl | | | |
| 32 | 1H-imidazol-2-yl | H | H | Et |
| 33 | thiophen-3-ylmethyl | H | H | Et |
| 34 | furan-2-ylmethyl | H | H | Et |
| 35 | quinolin-2-ylmethyl | H | H | Et |
| 36 | furan-3-ylmethyl | H | H | Et |
| 37 | (5-Me)-3H-imidazol-4- | H | H | Et |
| | ylmethyl | | | |
| 38 | (3-Me)-thiophen-2-yl | H | H | Et |
| 39 | 1H-imidazol-4-ylmethyl | Br | H | Et |
| 40 | thiophen-2-ylmethyl | Br | H | Et |
| 41 | phenethyl | H | H | Et |
| 42 | phenethyl | H | H | Et |
| 43 | cyanomethyl | phenyl | H | Et |
| 44 | 3-methyl-but-2-enyl | (3-carboxy)phenyl | H | Et |
| 45 | 3-methyl-but-2-enyl | (3-carboxy)phenyl | H | Et |
| 46 | 1H-imidazol-4-ylmethyl | H | H | Et |
| 47 | 3-methyl-but-2-enyl | (3-carboxy)phenyl | H | Et |
| 48 | H | H | Et | Et |
| 49 | H | Br | Et | Et |
| 50 | phenethyl | H | H | Me |
| 51 | phenethyl | H | Et | Et |
| 52 | thien-3-ylmethyl | H | Et | Et |
| 53 | n-butyl | H | H | Et |
| 54 | benzo[1,3]dioxol-5- | H | H | Et |
| | ylmethyl | | | |
| 55 | 3-methyl-but-2-enyl | H | H | Et |
| 56 | pyridin-2-ylmethyl | H | H | Et |
| 57 | pyridin-3-ylmethyl | H | H | Et |
| 58 | pyridin-4-ylmethyl | H | H | Et |
| 59 | 3-phenyl-prop-2-ynyl | H | H | Et |
| 60 | pyridin-2-ylmethyl | H | H | Me |
| 61 | thiophen-2-ylmethyl | H | H | Et |
| 62 | phenethyl | H | H | Et |
| 63 | 3-methyl-but-2-enyl | pyridin-4-yl | H | Et |
| 64 | thiophen-2-ylmethyl | quinolin-3-yl | H | Et |
| 65 | benzo[1,3]dioxol-5- | quinolin-3-yl | H | Et |
| | ylmethyl | | | |
| 66 | pyridin-2-ylmethyl | quinolin-3-yl | H | Et |
| 67 | 3-methyl-but-2-enyl | quinolin-8-yl | H | Et |
| 68 | thiophen-2-ylmethyl | quinolin-8-yl | H | Et |
| 69 | benzo[1,3]dioxol-5- | quinolin-8-yl | H | Et |
| | ylmethyl | | | |
| 70 | pyridin-2-ylmethyl | quinolin-8-yl | H | Et |
| 71 | quinolin-2-ylmethyl | pyridin-3-yl | H | Et |
| 72 | quinolin-2-ylmethyl | (3-N-acetamido) | H | Et |
| | | phenyl | | |
| 73 | quinolin-2-ylmethyl | (3-acetyl)phenyl | H | Et |
| 74 | (5-NO₂)-thiophen-3-yl | pyridin-3-yl | H | Et |
| 75 | (5-NO₂)--thiophen-3-yl | (3-N-acetamido) | H | Et |
| | | phenyl | | |
| 76 | (5-NO₂)--thiophen-3-yl | (3-acetyl)phenyl | H | Et |
| 77 | (5-Cl)-thiophen-2-yl | H | H | Et |
| 78 | (3-Me)- | H | H | Et |
| | benzothiophen-2-yl | | | |
| 79 | quinolin-2-ylmethyl | phenyl | H | Et |
| 80 | furan-2-ylmethyl | (3-carboxy)phenyl | H | Et |
| 81 | furan-3-ylmethyl | (3-carboxy)phenyl | H | Et |
| 82 | pyridin-2-ylmethyl | (3-carboxy)phenyl | H | Et |
| 83 | phenethyl | (3-carboxy)phenyl | H | Et |
| 84 | (4-N-acetamido) | (3-carboxy)phenyl | H | Et |
| | phenylmethyl | | | |
| 85 | quinolin-2-ylmethyl | (3-carboxy)phenyl | H | Et |
| 86 | (2-OH)phenethyl | H | H | Et |
| 87 | (5-carboxy)-furan-2-yl | (3-carboxy)phenyl | H | Et |
| 88 | (4-(44-Cl)-phenyl- | phenyl | H | Et |
| | carbonyl)-N—Me-pyrrol- | | | |
| | 2-yl-carbonylmethyl | | | |
| 89 | phenethyl | Br | Et | Et |
| 90 | 2H-tetrazol-5-ylmethyl | phenyl | H | Et |
| 91 | 2-(4- | phenyl | H | Et |
| | methoxycarbonylpipera- | | | |
| | zin-1-yl)eth-1-yl | | | |
| 92 | H | Br | H | Et |
| 93 | (3-carboxy)- | phenyl | H | Et |
| | phenylmethyl | | | |
| 94 | (4-carboxy) | phenyl | H | Et |
| | phenylmethyl | | | |
| 95 | (5-carboxy)-furan-2-yl | phenyl | H | Et |
| 96 | furan-2-ylmethyl | (4-carboxy)phenyl | H | Et |
| 97 | furan-3-ylmethyl | (4-carboxy)phenyl | H | Et |
| 98 | pyridin-2-ylmethyl | (4-carboxy)phenyl | H | Et |
| 99 | phenethyl | (4-carboxy)phenyl | H | Et |
| 100 | quinolin-2-ylmethyl | (4-carboxy)phenyl | H | Et |
| 101 | quinolin-2-ylmethyl | pyrimidin-5-yl | H | Et |
| 102 | thiazol-2-ylmethyl | (3-carboxy)phenyl | H | Et |
| 103 | H | H | H | Me |
| 104 | methyl | H | H | Me |
| 105 | cyclopropylmethyl | H | H | Me |
| 106 | 3-cyano-3,3- | H | H | Et |
| | diphenylprop-1-yl | | | |
| 107 | 3-dimethylamino | H | H | Et |
| | carbonyl-3,3- | | | |
| | diphenylprop-1-yl | | | |
| 108 | furan-2-ylmethyl | (3-amino-5- | H | Et |
| | | carboxy)phenyl | | |
| 109 | furan-2-ylmethyl | (4-C(═O)NEt₂)phenyl | H | Et |
| 110 | furan-3-ylmethyl | (3-amino-5- | H | Et |
| | | carboxy)phenyl | | |
| 111 | furan-3-ylmethyl | (4-C(═O)NEt₂)phenyl | H | Et |
| 112 | pyridin-2-ylmethyl | (3-amino-5- | H | Et |
| | | carboxy)phenyl | | |
| 113 | pyridin-2-ylmethyl | (4-C(═O)NEt₂)phenyl | H | Et |
| 114 | 3-methyl-but-2-enyl | (3-amino-5- | H | Et |
| | | carboxy)phenyl | | |
| 115 | 3-methyl-but-2-enyl | (4-C(O)NEt₂)phenyl | H | Et |
| 116 | phenethyl | (3-amino-5- | H | Et |
| | | carboxy)phenyl | | |
| 117 | phenethyl | (4-C(O)NEt₂)phenyl | H | Et |
| 118 | thiazol-2-ylmethyl | (3-amino-5- | H | Et |
| | | carboxy)phenyl | | |
| 119 | thiazol-2-ylmethyl | (4-C(O)NEt₂)phenyl | H | Et |
| 120 | thiophen-2-ylmethyl | (3-amino-5- | H | Et |
| | | carboxy)phenyl | | |
| 121 | thiophen-2-ylmethyl | (4-C(O)NEt₂)phenyl | H | Et |
| 122 | thiophen-3-ylmethyl | (4-C(O)NEt₂)phenyl | H | Et |
| 123 | furan-2-ylmethyl | (4-NO₂)phenyl | H | Et |
| 124 | furan-2-ylmethyl | 4(2-carboxy-2- | H | Et |
| | | aminoethy-1- | | |
| | | yl))phenyl | | |
| 125 | furan-2-ylmethyl | (2-carboxy-eth-1- | H | Et |
| | | yl)phenyl | | |
| 126 | furan-3-ylmethyl | (4-NO₂)phenyl | H | Et |
| 127 | furan-3-ylmethyl | 4-(2-carboxy-eth-1- | H | Et |
| | | yl)phenyl | | |
| 128 | thiophen-3-ylmethyl | (4-NO₂)phenyl | H | Et |
| 129 | thiophen-3-ylmethyl | 4-(2-carboxy-eth-1- | H | Et |
| | | yl)phenyl | | |
| 130 | thiazol-2-ylmethyl | (4-NO₂)phenyl | H | Et |
| 131 | thiazol-2-ylmethyl | 4-(2-carboxy-2- | H | Et |
| | | amino-eth-1- | | |
| | | yl)phenyl | | |
| 132 | thiazol-2-ylmethyl | 4-(2-carboxy-eth-1- | H | Et |
| | | yl)phenyl | | |
| 133 | thiazol-2-ylmethyl | H | H | Et |
| 134 | phenethyl | (4-NO₂)phenyl | H | Et |
| 135 | phenethyl | 4-(2-carboxy-eth-1- | H | Et |
| | | yl)phenyl | | |
| 136 | 3-methyl-but-2-enyl | (4-NO₂)phenyl | H | Et |
| 137 | 3-methyl-but-2-enyl | 4-(2-carboxy-eth-1- | H | Et |
| | | yl)phenyl | | |
| 138 | furan-3-ylmethyl | (4-SO₂NH₂)phenyl | H | Et |
| 139 | thiophen-3-ylmethyl | (4-SO₂NH₂)phenyl | H | Et |
| 140 | thiazol-2-ylmethyl | (4-SO₂NH₂)phenyl | H | Et |
| 141 | thiophen-2-ylmethyl | (4-SO₂NH₂)phenyl | H | Et |
| 142 | 3-methyl-but-2-enyl | (4-SO₂NH₂)phenyl | H | Et |
| 143 | furan-3-ylmethyl | CN | H | Et |
| 144 | furan-3-ylmethyl | 1H-tetrazol-5-yl | H | Et |
| 145 | H | CN | H | Et |
| 146 | furan-2-ylmethyl | 1H-tetrazol-5-yl | H | Et |
| 147 | 3-methyl-but-2-enyl | 1H-tetrazol-5-yl | H | Et |
| 148 | thiophen-3-ylmethyl | 1H-tetrazol-5-yl | H | Et |
| 149 | phenethyl | 1H-tetrazol-5-yl | H | Et |
| 150 | thiazol-2-ylmethyl | 1H-tetrazol-5-yl | H | Et |
| 151 | H | 1H-tetrazol-5-yl | H | Et |
| 152 | furan-3-ylmethyl | (3-carboxy-5- | H | Et |
| | | NO₂)phenyl | | |
| 153 | furan-3-ylmethyl | 3- | H | Et |
| | | (aminomethyl)phenyl | | |
| 154 | pyridin-2-ylmethyl | (3-carboxy-5- | H | Et |
| | | NO₂)phenyl | | |
| 155 | 3-methyl-but-2-enyl | (3-carboxy-5- | H | Et |
| | | NO₂)phenyl | | |
| 156 | 3-methyl-but-2-enyl | (3- | H | Et |
| | | aminomethyl)phenyl | | |
| 157 | phenethyl | (3-carboxy-5- | H | Et |
| | | NO₂)phenyl | | |
| 158 | phenethyl | (3- | H | Et |
| | | aminomethyl)phenyl | | |
| 159 | thiazol-2-ylmethyl | (3-carboxy-5- | H | Et |
| | | NO₂)phenyl | | |
| 160 | thiazol-2-ylmethyl | (3- | H | Et |
| | | aminomethyl)phenyl | | |
| 161 | thiophen-3-ylmethyl | (3-carboxy-5- | H | Et |
| | | NO₂)phenyl | | |
| 162 | thiophen-3-ylmethyl | (3- | H | Et |
| | | aminomethyl)phenyl | | |
| 163 | 3-methyl-but-2-enyl | 3-(1H-tetrazol-4- | H | Et |
| | | yl)phenyl | | |
| 164 | furan-2-ylmethyl | 3-(1H-tetrazol-4- | H | Et |
| | | yl)phenyl | | |
| 165 | pyridin-2-ylmethyl | 3-(1H-tetrazol-4- | H | Et |
| | | yl)phenyl | | |
| 166 | phenethyl | 3-(1H-tetrazol-4- | H | Et |
| | | yl)phenyl | | |
| 167 | thiazol-2-ylmethyl | 3-(1H-tetrazol-4- | H | Et |
| | | yl)phenyl | | |
| 168 | thiophen-2-ylmethyl | 3-(1H-tetrazol-4- | H | Et |
| | | yl)phenyl | | |
| 169 | thiophen-3-ylmethyl | 3-(1H-tetrazol-4- | H | Et |
| | | yl)phenyl | | |
| 170 | furan-3-ylmethyl | 3-(1H-tetrazol-4- | H | Et |
| | | yl)phenyl | | |
| 171 | ethylcarboxy | Br | H | Et |
| 172 | ethylcarboxy | (4-OH,3- | H | Et |
| | | OMe)phenyl | | |
| 173 | ethylcarboxy | (4-OH,3,5- | H | Et |
| | | dimethyl)phenyl | | |
| 174 | thiazol-2-ylmethyl | (4-carboxy)phenyl | H | Et |
| 175 | thiophen-3-ylmethyl | (3-carboxy)phenyl | H | Et |
| 176 | thiophen-3-ylmethyl | (4-carboxy)phenyl | H | Et |
| 177 | furan-3-ylmethyl | (4-C(═O)NH2)phenyl | H | Et |
| 178 | furan-3-ylmethyl | (3- | H | Et |
| | | hydroxymethyl)phenyl | | |
| 179 | furan-2-ylmethyl | (3- | H | Et |
| | | hydroxymethyl)phenyl | | |
| 180 | furan-2-ylmethyl | (4-C(═O)NH₂)phenyl | H | Et |
| 181 | pyridin-2-ylmethyl | (3- | H | Et |
| | | hydroxymethyl)phenyl | | |
| 182 | pyridin-2-ylmethyl | (4-NHSO₂Me)phenyl | H | Et |
| 183 | pyridin-2-ylmethyl | (4-C(═O)NH₂phenyl | H | Et |
| 184 | phenethyl | (3- | H | Et |
| | | hydroxymethyl)phenyl | | |
| 185 | phenethyl | (4-NHSO₂Me)phenyl | H | Et |
| 186 | thiazol-2-ylmethyl | (3- | H | Et |
| | | hydroxymethyl)phenyl | | |
| 187 | thiazol-2-ylmethyl | (4-NHSO₂Me)phenyl | H | Et |
| 188 | thiazol-2-ylmethyl | (4-C(═O)NH₂phenyl | H | Et |
| 189 | thiophen-3-ylmethyl | (3- | H | Et |
| | | hydroxymethyl)phenyl | | |
| 190 | thiophen-3-ylmethyl | (4-NHSO₂Me)phenyl | H | Et |
| 191 | thiophen-3-ylmethyl | (4-C(═O)NH₂)phenyl | H | Et |
| 192 | furan-2-ylmethyl | (4- | H | Et |
| | | hydroxymethyl)phenyl | | |
| 193 | pyridin-2-ylmethyl | (4- | H | Et |
| | | hydroxymethyl)phenyl | | |
| 194 | 3-methyl-but-2-enyl | (4- | H | Et |
| | | hydroxymethyl)phenyl | | |
| 195 | thiazol-2-ylmethyl | (4- | H | Et |
| | | hydroxymethyl)phenyl | | |
| 196 | thiophen-3-ylmethyl | (4- | H | Et |
| | | hydroxymethyl)phenyl | | |
| 197 | cyano | phenyl | Et | Et |
| 198 | aminoiminomethyl | phenyl | Et | Et |
| 199 | formyl | phenyl | Et | Et |
| 200 | benzylcarbonyl | pyridin-3-yl | H | Et |
| 201 | H | 1H-tetrazol-5-yl | H | Et |
| 202 | H | 1H-tetrazol-5-yl | H | Et |
| 203 | furan-2-ylmethyl | 1H-tetrazol-5-yl | H | Et |
| 204 | furan-2-ylmethyl | 1H-tetrazol-5-yl | H | Et |
| 205 | thien-3-ylmethyl | (4- | H | Et |
| | | hydroxymethyl)phenyl | | |
| 206 | thien-3-ylmethyl | (4- | H | Et |
| | | hydroxymethyl)phenyl | | |
| 207 | formyl | pyridin-3-yl | H | Et |
| 208 | thien-2-ylcarbonyl | pyridin-3-yl | H | Et |
| 209 | furan-3-ylmethyl | CN | H | Et |
| 210 | furan-3-ylmethyl | CN | H | Et |
| 211 | furan-3-ylmethyl | Br | H | Et |
| 212 | furan-3-ylmethyl | Br | H | Et |
| 213 | pyridin-2-ylcarbonyl | pyridin-3-yl | H | Et |
| 214 | furan-3-ylcarbonyl | pyridin-3-yl | H | Et |
| 215 | thiophen-3-ylmethyl | 3-(1H-tetrazol-4- | H | Et |
| | | yl)phenyl | | |
| 216 | thiophen-3-ylmethyl | 3-(1H-tetrazol-4- | H | Et |
| | | yl)phenyl | | |
| 217 | benzylcarbonyl | pyridin-3-yl | H | Et |
| 218 | benzylcarbonyl | pyridin-3-yl | H | Et |
| 219 | pyridin-2-ylmethyl | Br | H | Et |
| 220 | pyridin-2-ylmethyl | Br | H | Et |
| 221 | thiophen-3-ylmethyl | Br | H | Et |
| 222 | thiophen-3-ylmethyl | Br | H | Et |
| 223 | pyridin-2-ylmethyl | (4-C(O)NEt₂)phenyl | H | Et |
| 224 | pyridin-2-ylmethyl | (4-C(O)NEt₂)phenyl | H | Et |
| 225 | 3-methyl-but-2-enyl | Br | H | Et |
| 226 | 3-methyl-but-2-enyl | Br | H | Et |

and pharmaceutically acceptable enantiomers, diastereomers and salts thereof.

Instant compounds of the invention may also be present in the form of a pharmaceutically acceptable salts. The pharmaceutically acceptable salt generally takes a form in which the basic nitrogen is protonated with an inorganic or organic acid. Representative organic or inorganic acids include hydrochloric, hydrobromic, hydroiodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharic or trifluoroacetic.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The compounds of this invention are chiral and, thus, may exist as enantiomers. In addition, the compounds may exist as diastereomers. It is to be understood that all such enantiomers and diastereomers, as well as all mixtures thereof, are encompassed within the scope of the present invention.

Furthermore, some of the crystalline forms for the compounds may exist as polymorphs and as such are intended to be included in the present invention.

In addition, some of the compounds may form solvates with water (i.e., hydrates) or common organic solvents, and such solvates are also intended to be encompassed within the scope of this invention.

The present invention also contemplates a pharmaceutical composition comprising a combination of a δ-opioid or μ-opioid receptor modulator compound of Formula (I) and a μ-opioid receptor modulator compound known to those skilled in the art or a δ-opioid or μ-opioid receptor modulator compound of Formula (I) wherein the combination has a synergistic therapeutic effect.

Suitable μ-opioid receptor modulator compounds known to those skilled in the art for use in such a combination include, without limitation, the compounds alfentanil, allylprodine, alphaprodine, anileridine, bezitramide, buprenorphine, clonitazene, cyclazocine, dextromoramide, dihydrocodeine, dihydromorphine, ethoheptazine, ethylmorphine, etonitazene, fentanyl, heroin, hydromorphone, hydroxypethidine, isomethadone, ketobemidone, levallorphan, levorphanol, lofentanil, meperidine, meptazinol, metazocine, methadone, morphine, nalbuphine, norlevorphanol, normethadone, nalorphine, normorphine, opium, oxycodone, oxymorphone, phenazocine, piritramide, propiram, propoxyphene, sufentanil, tramadol and diastereomers, salts, complexes and mixtures thereof of any of the foregoing.

The terms used in describing the invention are commonly used and known to those skilled in the art. However, the terms that could have other meanings are hereinafter defined. These definitions apply to the terms as they are used throughout this specification, unless otherwise limited in specific instances, either individually or as part of a larger group.

An “independently” selected substituent refers to a group of substituents, wherein the substituents may be different. Therefore, designated numbers of carbon atoms (e.g., C₁-C₆) shall refer independently to the number of carbon atoms in an alkyl or cycloalkyl moiety or to the alkyl portion of a larger substituent in which alkyl appears as its prefix root.

The term “alkyl” refers to straight and branched-chain alkyl radical groups with 1 to 8 carbon atoms or any number within this range. The terms “alkenyl” and “alkynyl” refer to radical groups having straight and branched chains with 2 to 8 carbon atoms or any number within this range. For alkenyl chains, one double bond is formed between adjacent members of a two or three carbon chain and one or two double bonds are formed between adjacent members of a four to eight carbon chain. For alkynyl chains, one triple bond is formed between adjacent members of a two or three carbon chain and one or two triple bonds are formed between adjacent members of a four to eight carbon chain. Correspondingly, the terms “alkylene,” “alkenylene” and “alkynylene” refer to alkyl, alkenyl and alkynyl linking groups wherein alkyl, alkenyl and alkynyl are as defined supra. Preferably, alkenylene and alkynylene linking group chains have at least one saturated carbon atom on each side of the unsaturated bond. More preferably, when an aryl or heteroaryl substituent is attached to the terminal carbon of an alkenylene or alkynylene linking group, at least one saturated carbon atom is between the unsaturated bond and the substituent. The term “alkoxy” refers to O-alkyl groups wherein alkyl is as defined supra.

Whenever the term “alkyl” appears in the name of a substituent (e.g., hydroxy(C₁₋₆)alkyl) it shall be interpreted as including those limitations given above for “alkyl.” Designated numbers of carbon atoms (e.g., C₁₋₆) shall refer independently to the number of carbon atoms in an alkyl or cycloalkyl moiety or to the alkyl portion of a larger substituent in which alkyl appears as its prefix root.

The term “cycloalkyl” refers to branched or unbranched cyclic aliphatic hydrocarbon chains of three to seven carbon atom members. Examples of such cyclic alkyl rings include cyclopropyl, cyclobutyl, cyclopentyl, cyclohexyl or cycloheptyl.

The term “heterocyclyl” refers to a nonaromatic cyclic ring of five to ten members in which one to four members are nitrogen or a nonaromatic cyclic ring of five to seven members in which zero, one or two members are nitrogen and one member is oxygen or sulfur; and in which,
a) optionally, the ring contains zero, one or two unsaturated bonds;
b) optionally, up to three carbon members adjacent to nitrogen members may be oxo substituted.

Optionally, the heterocyclyl ring is fused:
a) to a benzene ring;
b) to a 5 or 6 membered heteroaryl containing one of O, S or N and, optionally, one additional nitrogen;
c) to a 5 to 7 membered alicyclic ring;
d) to a 5 to 7 membered heterocyclyl ring of the same definition as above but absent the option of a further fused ring.

For instant compounds of the invention, the carbon atom ring members that form the heterocyclyl ring are fully saturated. Other compounds of the invention may have a partially saturated heterocyclyl ring. Preferred partially unsaturated heterocyclyl rings may have one or two double bonds. Such compounds are not considered to be fully aromatic and are not referred to as heteroaryl compounds. Therefore, a five member heterocyclyl ring may optionally have a double bond formed in the ring between adjacent ring members; a six or seven member heterocyclyl ring may have two double bonds formed in the ring between adjacent ring members.

The term aryl refers to a single aromatic ring of six carbon members or a bicyclic aromatic ring of ten carbon members. Examples of such aryl rings include phenyl and naphthyl.

The term heteroaryl refers to an aromatic ring of five to ten members wherein the ring has at least one heteroatom member. Suitable heteroatoms include nitrogen, oxygen or sulfur. In the case of five-membered rings, the heteroaryl ring contains one member of nitrogen, oxygen or sulfur and, in addition, may contain up to three additional nitrogens. In the case of six-membered rings, the heteroaryl ring may contain from one to three nitrogen atoms. For the case wherein the six member ring has three nitrogens, at most two nitrogen atoms are adjacent.

The terms “halo₁₋₃(C₁₋₈)alkyl,” “cycloalkyl(C₁₋₈)alkyl” or “hydroxy(C₁₋₆)alkyl” refer to an alkylene group substituted at the terminal carbon with a halo, cycloalkyl or hydroxy group, respectively. Similarly, the term “C₁₋₈alkoxy(C₁₋₈)alkenyl” or “C₁₋₈alkoxy(C₁₋₈)alkynyl” refers to an alkenylene or alkynylene group substituted at the terminal carbon with an alkoxy group. The term “carbonyl” refers to the linking group —C═O—. Furthermore, the term “methylenedioxy” refers to the substituent moiety —OCH₂O—, the term “ethylenedioxy” refers to the substituent moiety —O(CH₂)₂O— and the term “trimethylenedioxy” refers to the substituent moiety —O(CH₂)₃O—. The term “hydroxy” refers to the group —OH and the term “oxo” refers to the group=O. The term “halo” or “halogen” refers to the group iodine, bromine, chlorine and fluorine.

Where the compounds according to this invention are chiral, they may accordingly exist as enantiomers. In addition, the compounds may exist as diastereomers. It is to be understood that all such isomers and mixtures thereof are encompassed within the scope of the present invention.

The terms used in describing the invention are commonly used and known to those skilled in the art. As used herein, the following abbreviations have the indicated meanings:

| | |
|---|---|
| AcOH = | acetic acid |
| BOC or Boc = | t-butoxycarbonyl |
| BSA = | bovine serum albumin |
| DCE = | dichloroethane |
| DCM = | dichloromethane |
| DEA = | diethylamine |
| DIC = | diisopropylcarbodiimide |
| DIPEA = | diisopropylethylamine |
| DMAP = | 4-N,N-dimethylaminopyridine |
| DME = | 1,2-dimethoxyethane |
| DMF = | dimethyl formamide |
| Et = | ethyl |
| EtOAc = | ethyl acetate |
| EtOH = | ethanol |
| Et₂O = | diethyl ether |
| Fmoc = | 9H-fluoren-9-ylmethoxycarbonyl |
| FMPB = | 4-(4-formyl-3-methoxyphenoxy)butyryl AM resin |
| h = | hour/hours |
| HEPES = | 4-(2-hydroxyethyl)-1-piperizine ethane |
| | sulfonic acid |
| HATU = | O-(7-azabenzotriazol-1-yl)-N,N,N″,N″- |
| | tetramethyl uronium hexafluorophosphate |
| HOAT = | 1-hydroxy-7-azabenzotriazole |
| HOBT = | 1-hydroxybenzotriazole |
| LDA = | lithium diisopropylamide |
| Me = | methyl |
| MEK = | methyl ethyl ketone |
| MeOH = | methanol |
| min = | minute/minutes |
| Na(OAc)₃BH = | sodium triacetoxyborohydride |
| NMP = | N-methyl-2-pyrrolidinone |
| Ph = | phenyl |
| RT or rt = | room temperature |
| TEA = | triethylamine |
| TFA = | trifluoroacetic acid |
| THF = | tetrahydrofuran |
| TMOF = | trimethylorthoformate |

### General Synthetic Methods

Representative compounds of the present invention can be synthesized in accordance with the general synthetic methods described below and are illustrated in the schemes that follows. Since the schemes are an illustration, the invention should not be construed as being limited by the chemical reactions and conditions expressed. The preparation of the various starting materials used in the schemes is well within the skill of persons versed in the art.

Scheme 1 describes a general scheme for the preparation of certain target 3-(diarylmethylene)-8-azabicyclo[3.2.1]octane derivatives of the invention using synthetic methods to prepare intermediate compounds also intended to be within the scope of the present invention.

A Suzuki reaction is used to couple a boronic acid Compound 1a with an iodinated Compound 1b in the presence of carbon monoxide to produce an intermediate Compound 1c. Alternatively, Compound 1b may also be substituted with bromine or OTf (trifluoromethylsulfonyloxy) in place of iodine. For Compound 1a and Compound 1b, the R² substituent and -A-Z moiety may be varied by using appropriate starting materials or may be added in later steps.

For example, the -Z- portion of the —C(-Z moiety may be varied using —OH, —O(alkyl) or —N(R⁵)(R⁶) to produce other intermediate compounds of the present invention. Similarly, target compounds wherein Z is —O(R⁴) and R⁴ is hydrogen may be conveniently produced by conventional hydrolysis of the Z is —N(R⁵)(R⁶) group; furthermore, other compounds wherein Z is —O(R⁴) and R⁴ is hydrogen may be esterified by conventional methods to produce other target compounds wherein R⁴ is C₁₋₈alkyl.

A Robinson-Schöpf condensation is used to prepare tropinone intermediate Compounds 1e bearing an R¹ substituent on nitrogen by mixing an R¹ substituted amine Compound 1e with a succinaldehyde precursor such as 2,5-dimethoxytetrahydrofuran and acetonedicarboxylic acid. For a Compound 1e, the R¹ substituent may be varied by using appropriate starting materials or may be added in later steps.

Compound 1c and Compound 1e may be coupled using a titanium mediated “McMurray” reaction to produce a target Compound 1f.

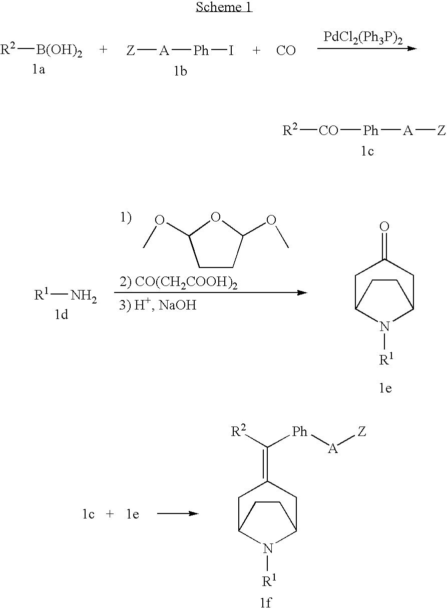

Scheme 2 describes another general scheme for the preparation of certain 3-(diarylmethylene)-8-azabicyclo[3.2.1]octane derivatives.

As shown below in Scheme 2, the intermediate Compound 1c may be coupled with an 8-methyl-8-azabicyclo[3:2:1]octanone compound using titanium mediated coupling to produce an intermediate Compound 2a.

The intermediate Compound 2a may be treated with 2,2,2-trichloroethyl chloroformate followed by reflux with zinc powder in MeOH to obtain the N-demethylated Compound 2b. Compound 2c is produced by alkylation of Compound 2b with an alkyl halide or reductive alkylation with sodium triacetoxyborohydride and a carbonyl compound.

As desired, the identity of the -A-Z moiety may be varied by conversion of one -A-Z moiety to another. For example, an -A-Z moiety where the -A-portion is —C(═O)— and the -Z- portion is —O(alkyl), the -Z- portion may be hydrolyzed to the acid, wherein —O(alkyl) becomes —OH. Subsequently, the resulting carboxyl group may be converted to the desired amide; and, conversely, an amide group may be hydrolyzed to an acid.

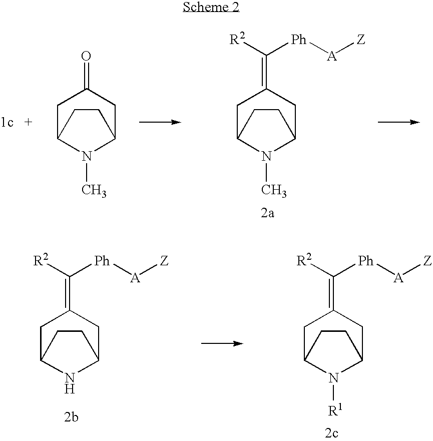

As shown in Scheme 3, a Compound 3a wherein X is 0 may also be further treated with a suitable thionating agent such as P₂S₅ or Lawesson's Reagent to prepare a Compound 3b wherein X is S.

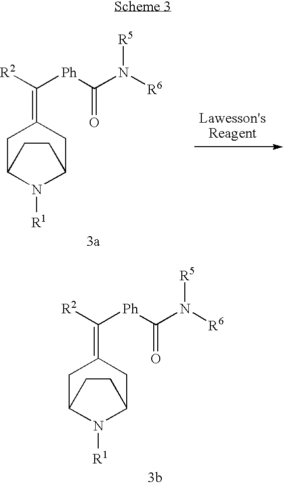

Scheme 4 illustrates the synthesis of compounds of the present invention wherein R² may be hydrogen or bromide and A is defined as —C(═O)—. A Wittig reaction is used to condense Compound 4b with tropinone intermediate Compound 1e to form a tropanildene Compound 4c, wherein R² is hydrogen. Compound 4c may be reacted with bromine to yield Compound 4d wherein R² is bromine.

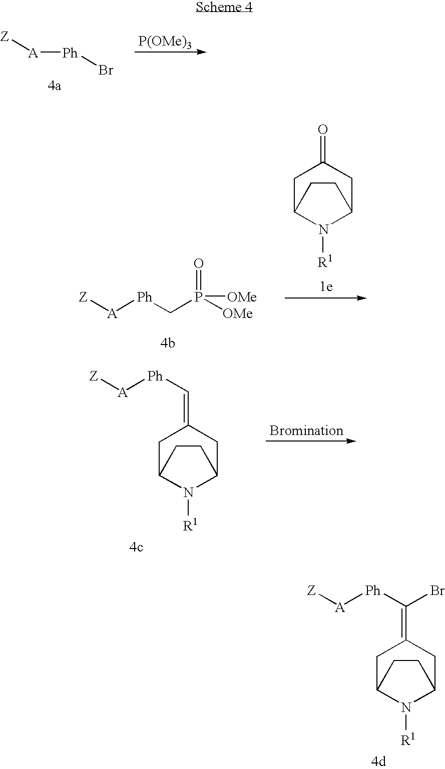

Compounds of the present invention may be made using solid phase synthesis as illustrated in Scheme 5, wherein substituent R¹ has been replaced with an Fmoc protecting group using chemistry known to those skilled in the art. An FMPB aldehyde resin may be reductively aminated with an amine, preferably ethylamine, and a hydride source such as sodium triacetoxyborohydride to give Compound 5b. Compound 5b may be coupled with Compound 4d (wherein -A-Z is —C(═O)OH) in the presence of a coupling agent such as 2-chloro-1,3-dimethylimidazoliium chloride to form resin-bound amide Compound 5c,

Compound 5c may be coupled with a boronic acid Compound 1a in the presence of a palladium catalyst to yield Compound 5d of the present invention. The Fmoc protecting group may be removed using standard procedures. Subsequently, the free amino group of Compound 5e may be substituted with R¹ substituents of the present invention using conventional alkylation methods such as reductive amination or reaction with alkyl halides to afford Compound 5f.

Compound 5e may also be acylated using conventional acylation methods, using such reagents as acid chlorides, anhydrides, isocyanates, or coupling with carboxylic acids in the presence of an appropriate coupling agent. Compound 5f may then be cleaved from the FMPB resin under acidic conditions to yield amide Compound 5g.

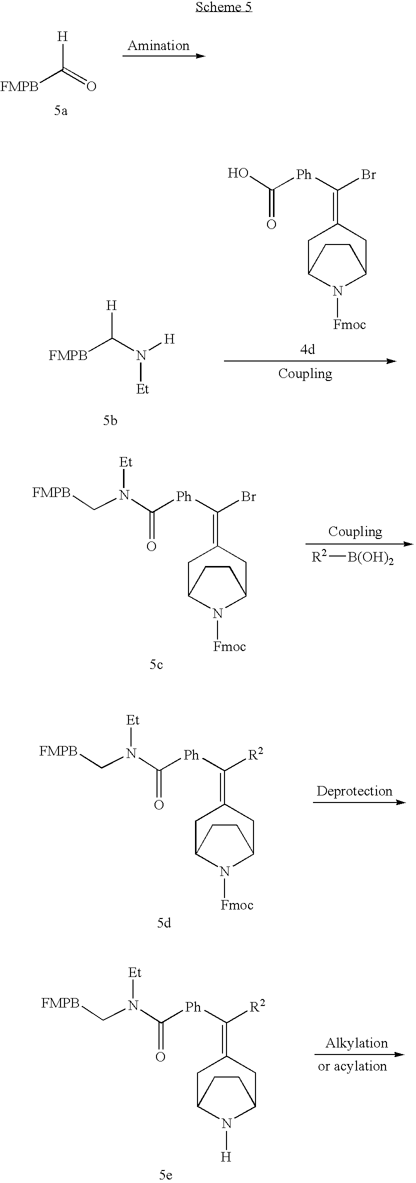

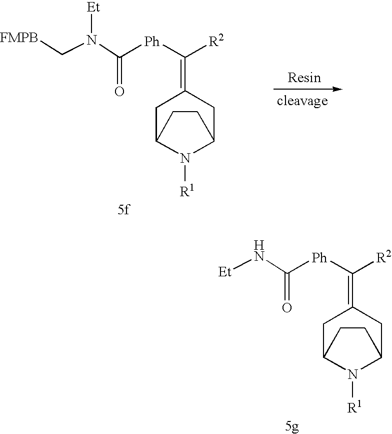

Similarly, Compound 5c may be converted to compounds of the present invention wherein R² is cyano by treatment with zinc cyanide in the presence of a palladium catalyst. Subsequently, the cyano substituent may be reacted with trimethylsilylazide in the presence of tin to form compounds of the present invention wherein R² is a tetrazolyl substituent.

The preparation of compounds of the present invention described in Scheme 5 is also amenable to solution phase synthesis.

As shown in Scheme 6, Compound 5g wherein R¹ may be treated with iodoacetonitrile to afford Compound 6a, wherein R¹ is cyanomethyl. Compound 6a may then be reacted with trimethylsilylazide in the presence of trimethylaluminum to afford Compound 6b wherein R¹ is tetrazolylmethyl.

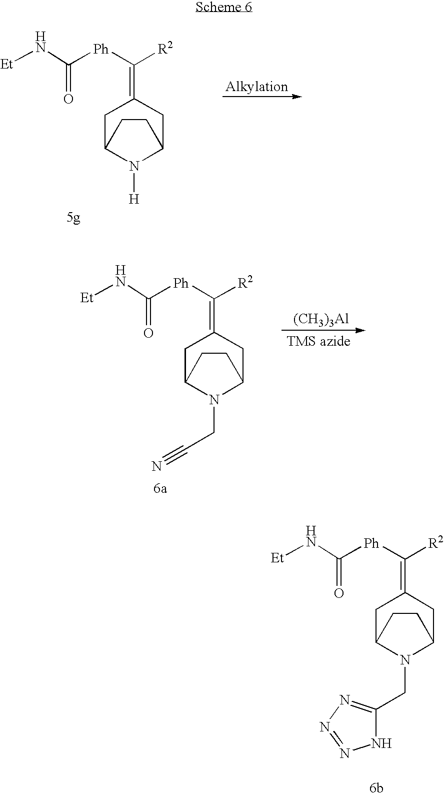

Scheme 7 describes the preparation of compounds of the present invention wherein R¹ is hydrogen. Compound 3a (wherein R¹ is ethylcarboxy) may be treated with trimethylsilyl iodide to yield tropanilidene Compound 6a.

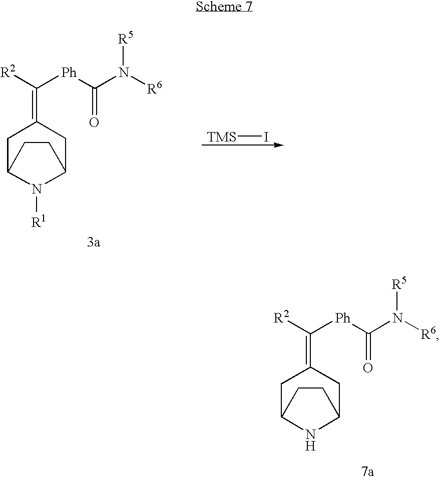

As shown in Scheme 8, Compound 7a may be reacted with cyanogen bromide to form Compound 8a, which is then treated with ammonium chloride in the presence of an aluminum reagent to form Compound 8b.

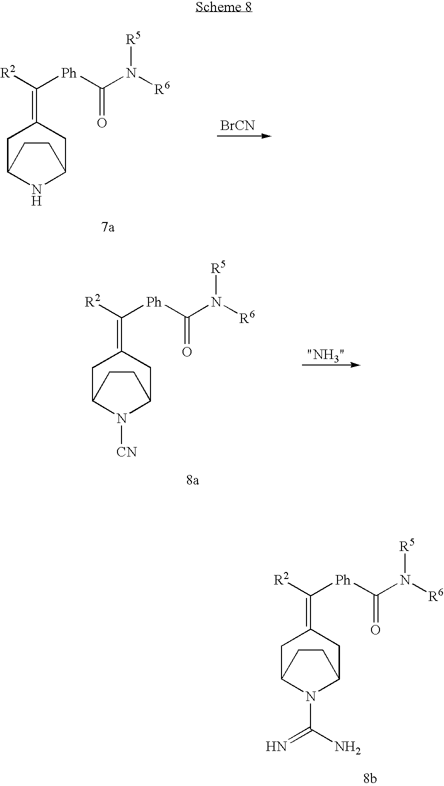

The compounds of the present invention may be used to treat mild to moderately severe pain in warm-blooded animals such as humans by administration of an analgesically effective dose. The dosage range would be from about 0.01 mg to about 15,000 mg, in particular from about 0.1 mg to about 3500 mg or, more particularly from about 0.1 mg to about 1000 mg of active ingredient in a regimen of about 1 to 4 times per day for an average (70 kg) human; although, it is apparent to one skilled in the art that the therapeutically effective amount for active compounds of the invention will vary as will the types of pain being treated.

Examples of pain intended to be within the scope of the present invention include, but are not limited to, centrally mediated pain, peripherally mediated pain, structural or soft tissue injury related pain, progressive disease related pain, neuropathic pain and acute pain such as caused by acute injury, trauma or surgery and chronic pain such as caused by neuropathic conditions, diabetic peripheral neuropathy, post-herpetic neuralgia, trigeminal neuralgia, post-stroke pain syndromes or cluster or migraine headaches.

In regard to the use of the present compounds as immunosuppressants, antiinflammatory agents, agents for the treatment of neurological and psychiatric conditions, medicaments for drug and alcohol abuse, agents for treating gastritis and diarrhea, cardiovascular agents and agents for the treatment of respiratory diseases, a therapeutically effective dose can be determined by persons skilled in the art by the use of established animal models. Such a dose would likely fall in the range of from about 0.01 mg to about 15,000 mg of active ingredient administered 1 to 4 times per day for an average (70 kg) human.

Pharmaceutical compositions of the invention comprise the formula (I) compounds as defined above, particularly in admixture with a pharmaceutically acceptable carrier. Illustrative of the invention, therefore, is a pharmaceutical composition comprising a pharmaceutically acceptable carrier and any of the compounds described above. Another illustration of the invention is a pharmaceutical composition made by mixing any of the compounds described above and a pharmaceutically acceptable carrier. A further illustration of the invention is a process for making a pharmaceutical composition comprising mixing any of the compounds described above and a pharmaceutically acceptable carrier.

To prepare the pharmaceutical compositions of this invention, one or more compounds of the invention or salt thereof, as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

### Specific Synthetic Methods

Specific compounds which are representative of this invention may be prepared as per the following examples offered by way of illustration and not by way of limitation. For the sake of clarity, bracketed numbers following compound names indicate the stoichiometric salt associated with the compound, which is further exemplified by the calculated analytical data. Also, examples specifically used to prepare intermediates for the further synthesis of compounds of the invention are designated by “Procedure.” As well, instant compounds may also be used as starting materials in subsequent examples to produce additional compounds of the present invention. No attempt has been made to optimize the yields obtained in any of the reactions. One skilled in the art would know how to increase such yields through routine variations in reaction times, temperatures, solvents and/or reagents.

### Procedure A

### N,N-Diethyl-4-benzoylbenzamide

A solution of 25 g (110 mmol) 4-benzoylbenzoic acid [611-95-0]and 20 mL SOCl₂ was allowed to reflux for 2 h then allowed to cool. The excess SOCl₂ was evaporated off and the resulting clear oil was dissolved in 10 mL CH₂Cl₂ then slowly added to 12 mL (116 mmol) diethylamine in a mixture of 10 mL 3N NaOH and 50 mL CH₂Cl₂. The mixture was allowed to stir for 30 min then partitioned between H₂O and CH₂Cl₂. The organic layer was washed with brine, dried over K₂CO₃, filtered and concentrated. The product precipitated from EtOAc/hexane to give 29.6 g (105 mmol) white crystals. MS m/z (MH⁺) 282.

### EXAMPLE 1

### N,N-Diethyl-4-[(8-methyl-8-azabicyclo[3.2.1]oct-3-ylidene)phenylmethyl]benzamide Hydrochloride [1:1]

A 100 mL dry THF slurry of 18.6 g (284 mmol) zinc powder and 15.6 mL (142 mmol) TiCl₄ was stirred and allowed to reflux for 2 h under Ar. The reaction was allowed to cool then a 20 mL THF solution of 10 g (35.5 mmol) N,N-diethyl-4-benzoylbenzamide and 5 g (35.5 mmol) tropinone was added slowly. Once the addition was complete, the reaction was allowed to reflux for 3 h, cooled, then quenched with 10% K₂CO₃ in H₂O. The resulting slurry was partitioned between water and Et₂O. The organic fraction was washed with brine, dried over MgSO₄, filtered, and concentrated. The remaining yellow oil was absorbed onto silica gel then purified by flash chromatography eluted with 10% 0.5 M NH₃ in MeOH 90% CH₂Cl₂ to produce the product N,N-diethyl-4-[(8-methyl-8-azabicyclo[3.2.1]oct-3-ylidene)phenylmethyl]benzamide (4.27 g, 11 mmol). The HCl salt was precipitated from Et₂O after the addition of ethereal HCl; mp 145-147° C. MS m/z (MH⁺) 389. ¹H NMR 300 MHz (DMSO-d₆) δ 7.2-7.45 (m, 9H), 3.8-3.9 (m, 2H), 3.15-3.25 (m, 2H), 2.75-2.95 (m, 4H), 2.65 (s, 3H), 2.25-2.4 (m, 2H), 2.15-2.25 (m, 2H), 1.75-1.9 (m, 2H), 0.95-1.2 (m, 6H). Anal calc C₂₆H₃₂N₂O.HCl (3% H₂O): C, 71.21; H, 7.93; N, 6.39. Found: C, 71.16; H, 7.95; N, 6.27.

### EXAMPLE 2

### N,N-Diethyl-4-[(8-azabicyclo[3.2.1]oct-3-ylidene)phenylmethyl]benzamide Hydrochloride [1:1]

A 100 mL benzene suspension of 3.1 g (5.6 mmol) N,N-diethyl-4-[(8-methyl-8-azabicyclo[3.2.1]oct-3-ylidene)phenylmethyl]benzamide, 3.45 g (25 mmol) K₂CO₃, and 1.5 mL (10 mmol) 2,2,2-trichloroethyl chloroformate was allowed to reflux for 2 h. The reaction was cooled, filtered, and the solvent evaporated. The residual oil was dissolved in MeOH then stirred at reflux with 2.6 g (40 mmol) zinc powder for 1 h. After cooling, the reaction was filtered through celite and partitioned between 3N NaOH and CH₂Cl₂. The organic layer was washed with brine, dried over K₂CO₃, filtered, and concentrated (2.1 g, 5.6 mmol). The resulting clear oil was dissolved in Et₂O, filtered, and the product precipitated after the addition of ethereal HCl; mp 128-132° C. MS m/z (MH⁺) 375. ¹H NMR 300 MHz (DMSO-d₆) δ 7.15-7.4 (m, 9H), 3.9-4.0 (m, 2H), 3.15-3.3 (m, 2H), 2.55-2.65 (m, 2H), 2.25-2.35 (m, 4H), 1.9-2.0 (m, 2H), 1.75-1.85 (m, 2H), 1.0-1.2 (m, 6H). Anal calc C₂₅H₃₀N₂O.HCl (3% H₂O): C, 70.89; H, 7.71; N, 6.61. Found: C, 70.52; H, 7.41; N, 6.24.

### EXAMPLE 3

### (+)—N,N-Diethyl-4-[[(1R,5S)-8-azabicyclo[3.2.1]oct-3-ylidene]phenylmethyl]benzamide Fumarate [1:1]

N,N-Diethyl-4-[(8-azabicyclo[3.2.1]oct-3-ylidene)phenylmethyl]benzamide was chromatographed on a CHIRALPAK® AS™ eluting with 90:9.9:0.1 acetonitrile:2-propanol:diethylamine. The first enantiomer to elute was converted to its fumarate salt in 2-PrOH. [α]_{D}²⁵=+29°. MS m/z (MH⁺) 375.

### EXAMPLE 4

### (−)—N,N-Diethyl-4-[[(1R,5S)-8-azabicyclo[3.2.1]oct-3-ylidene]phenylmethyl]benzamide Fumarate [1:1]

The second enantiomer to elute in the chromatography from the foregoing example was collected. [α]_{D}²⁵=−220. MS m/z (MH⁺) 375.

### EXAMPLE 5

### N,N-Diethyl-4-[(8-allyl-8-azabicyclo[3.2.1]oct-3-ylidene)phenylmethyl]benzamide Hydrochloride [1:1]

A 20 mL acetonitrile suspension of 0.4 g (1.0 mmol) N,N-diethyl-4-[(8-azabicyclo[3.2.1]oct-3-ylidene)phenylmethyl]benzamide, 0.4 g (3.0 mmol) K₂CO₃, and 0.09 mL allyl bromide was allowed to stir for 3 h. The reaction was filtered and concentrated. The remaining oil was absorbed onto silica gel then purified by flash chromatography eluted with 5% 0.5 M NH₃ in MeOH 95% CH₂Cl₂. The pure product (0.2 g, 0.4 mmol) was taken up in Et₂O, filtered, and precipitated after the addition of ethereal HCl. MS m/z (MH⁺) 415. ¹H NMR 300 MHz (DMSO-d₆) δ 7.15-7.45 (m, 9H), 5.95-6.10 (m, 1H), 5.4-5.55 (m, 2H), 3.85-3.95 (m, 2H), 3.55-3.65 (t, 2H), 3.35-3.45 (m, 2H), 3.1-3.25 (m, 2H), 2.75-2.85 (t, 2H), 2.2-2.3 (m, 2H), 2.1-2.25 (m, 2H), 1.75-1.9 (m, 2H), 1.0-1.2 (m, 6H).

### EXAMPLE 6

### (−)—N,N-Diethyl-4-[[(1R,5S)-8-allyl-8-azabicyclo[3.2.1]oct-3-ylidene]phenylmethyl]benzamide Hydrochloride

Following the protocol for Example 5 and substituting (+)—N,N-diethyl-4-[[(1R,5S)-8-azabicyclo[3.2.1]oct-3-ylidene]phenylmethyl]benzamide for N,N-diethyl-4-[(8-azabicyclo[3.2.1]oct-3-ylidene)phenyl methyl]benzamide the title compound was obtained: MS m/z (MH⁺) 415. [α]_{D}²⁵=−3.8°. ¹H NMR 300 MHz (DMSO-d₆) δ 7.15-7.45 (m, 9H), 5.95-6.10 (m, 1H), 5.4-5.55 (m, 2H), 3.85-3.95 (m, 2H), 3.55-3.65 (t, 2H), 3.35-3.45 (m, 2H), 3.1-3.25 (m, 2H), 2.75-2.85 (t, 2H), 2.2-2.3 (m, 2H), 2.1-2.25 (m, 2H), 1.75-1.9 (m, 2H), 1.0-1.2 (m, 6H).

Examples 7-17
N,N-Diethyl-4-[(8-R¹-8-azabicyclo[3.2.1]oct-3-ylidene)phenylmethyl]benzamides
Following the procedure of Example 5 and substituting the appropriate
alkyl bromide for allyl bromide the following compounds were prepared:

| | | | MS m/z |
|---|---|---|---|
| Ex# | Alkyl bromide | R¹ | (MH⁺) |
| 7 | 2-(4-fluorophenyl)ethyl bromide | 2-(4-fluorophenyl)ethyl | 497 |
| 8 | 2-(2-thiophenyl)ethyl bromide | 2-(2-thiophenyl)ethyl | 485 |
| 9 | 3-(2-bromoethyl)indole | 2-(3-indolyl)ethyl | 518 |
| 10 | 1-bromo-2-cyclohexylethane | 2-cyclohexylethyl | 485 |
| 11 | 2-phenoxyethyl bromide | 2-phenoxyethyl | 495 |
| 12 | 1-(bromoethyl)-4-ethyl-1,4- | 2-(4-ethyl-5-oxo-1,4- | 515 |
| | dihydrotetrazol-5-one | dihydrotetrazol-1-yl)ethyl | |
| 13 | 2-bromo-1-phenylethanone | phenylcarbonylmethyl | 493 |
| 14 | 2-bromo-1-(4-methoxyphenyl)ethanone | (4-methoxyphenyl)carbonylmethyl | 523 |
| 15 | 2-bromo-1-(3-cyanophenyl)ethanone | (3-cyanophenyl)carbonylmethyl | 518 |
| 16 | 2-bromo-1-[3,4- | 3,4-(ethylenedioxy | 551 |
| | (ethylenedioxy)phenyl]ethanone | phenyl)carbonylmethyl | |
| 17 | 2-bromo-1-[3,4- | 3,4-(trimethylenedioxy | 565 |
| | (trimethylenedioxy)phenyl]-ethanone | phenyl)carbonylmethyl | |

### EXAMPLE 18

### N,N-Diethyl-4-[(8-propyl-8-azabicyclo[3.2.1]oct-3-ylidene)phenylmethyl]benzamide Hydrochloride [1:1]

A slurry of 0.4 g (1.0 mmol) N,N-diethyl-4-[(8-azabicyclo[3.2.1]oct-3-ylidene)phenylmethyl]benzamide, 0.11 mL (1.5 mmol) propionaldehyde, 0.1 mL (1.7 mmol) HOAc, and 0.5 g (2.3 mmol) NaBH(OAc)₃ in 20 mL DCE was allowed to stir for 16 h. The reaction was made strongly basic with 3N NaOH and diluted with CH₂Cl₂. The organic layer was separated, washed with brine, dried over K₂CO₃, filtered, and concentrated. The remaining oil was absorbed onto silica gel and purified by flash chromatography eluted with 5% 0.5 M NH₃ in MeOH 95% CH₂Cl₂. The pure product (0.25 g, 0.6 mmol) was taken up in Et₂O, filtered, and precipitated after the addition of ethereal HCl; mp 184-184° C. MS m/z (MH⁺) 417. ¹H NMR 300 MHz (CD₃OD) δ 7.2-7.45 (m, 9H), 3.95-4.05 (m, 2H), 3.45-3.6 (m, 2H), 3.2-3.3 (m, 2H), 2.95-3.05 (m, 2H), 2.55-2.7 (m, 4H), 2.2-2.3 (m, 2H), 1.95-2.05 (m, 2H), 1.7-1.85 (m, 2H), 1.0-1.35 (br m, 9H). Anal calc C₂₈H₃₆N₂O.HCl.0.5H₂O: C, 72.78; H, 8.29; N, 6.06. Found: C, 73.01; H, 7.94; N, 5.85.

### EXAMPLES 19-21

### N,N-Diethyl-4-[(8-R′-8-azabicyclo[3.2.1]oct-3-ylidene)phenylmethyl]benzamides

Following the procedure of Example 18 and substituting the appropriate carbonyl compound for propionaldehyde the following compounds were prepared:

| Ex# | Carbonyl Compound | R¹ | MS m/z (MH⁺) |
|---|---|---|---|
| 19 | phenylacetaldehyde | 2-phenylethyl | 479 |
| 20 | piperonal | piperonyl | 509 |
| 21 | hydrocinnamaldehyde | 3-phenylpropyl | 493 |

### Procedure B

### N-(3-Fluorophenyl)-N-methyl-3-benzoylbenzamide

Following Procedure A with the substitution of 20 g (88 mmol) 3-benzoylbenzoic acid [579-18-0]and 8.5 mL (88 mmol) 3-fluoroaniline for 4-benzoylbenzoic acid and diethyl amine, the product N-(3-fluorophenyl)-3-benzoylbenzamide was generated (28 g, 88 mmol) as a clear oil. The oil was dissolved in 50 mL dry THF to which a 10 mL THF slurry of 2.1 g (90 mmol) NaH was slowly added. The mixture was allowed to stir for 5 min then 5.6 mL (90 mmol) of MeI was added and continued stirring for 16 h. The reaction was carefully quenched with water and partitioned between water and CH₂Cl₂. The organic layer was washed with brine, dried over K₂CO₃, filtered, and concentrated to yield 29.3 g (88 mmol) product. MS m/z (MH⁺) 334.

### EXAMPLE 22

### N-(3-Fluorophenyl)-N-methyl-3-[(8-methyl-8-azabicyclo[3.2.1]oct-3-ylidene)phenylmethyl]benzamide Fumarate [1:1]

Following the procedure of Example 1 with the substitution of N-(3-fluorophenyl)-N-methyl-3-benzoylbenzamide obtained in Procedure B for N,N-diethyl-4-benzoylbenzamide, the product N-(3-fluorophenyl)-N-methyl-3-[(8-methyl-8-azabicyclo[3.2.1]oct-3-ylidene)phenylmethyl]benzamide was produced. The fumarate salt was precipitated from 2-PrOH/hexane, mp 122-125° C. MS m/z (MH⁺) 441. ¹H NMR 300 MHz (DMSO-d₆) δ 6.85-7.35 (m, 13H), 3.4 (s, 3H), 3.3-3.5 (m, 1H), 3.15-3.2 (m, 1H), 3.4-3.55 (m, 2H), 2.35 (s, 3H), 2.15-2.25 (m, 1H), 2.05-2.15 (m, 1H), 1.9-2.05 (m, 2H), 1.55-1.65 (m, 1H), 1.35-1.55 (br ms, 1H). Anal calc C₂₉H₂₉FN₂O.C₄H₄O₄: C, 71.21; H, 5.98; N, 5.03. Found: C, 71.50; H, 6.20; N, 4.92.

### EXAMPLE 23

### (−)—N,N-Diethyl-4-[[(1R,5S)-8-phenethyl-8-azabicyclo[3:2:1]oct-3-ylidene]phenylmethyl]benzamide Hydrochloride [1:1]

A suspension of 52 g (0.8 mole) of zinc powder and 800 mL of THF was cooled in an ice bath 44 mL (0.4 mole) of TiCl₄ was added dropwise with stirring. The ice bath was removed and the reaction refluxed for 2 h. A solution of 26.45 g (0.094 mole) of N,N-diethyl-4-benzoylbenzamide and 23.9 g (0.094 mole) of 8-phenethyl-8-azabicyclo[3.2.1]octan-3-one, in 100 mL of THF was added dropwise and the reaction was refluxed 4h. After cooling, the reaction mixture was poured into a beaker containing excess K₂CO₃ and ice. The mixture was extracted with ether, washed with brine, dried (K₂CO₃) and concentrated. There was obtained 47 g (˜0.1 mol) of crude (±)—N,N-diethyl-4-[(8-phenethyl-8-azabicyclo[3:2:1]oct-3-ylidene)phenylmethyl]benzamide as an oil. A sample of the oil and 38.33 g (0.1 mole) of (+)-ditoluoyl-D-tartaric acid were combined in 600 mL of acetonitrile. The solid was collected and recrystallized twice from acetonitrile. The solid was collected and partitioned between dilute sodium hydroxide and CH₂Cl₂. The organic solution was dried (K₂CO₃) and concentrated. The residue was converted to a hydrochloride salt (Et₂O/HCl). It was recrystallized from 2-PrOH to give 5.6 g of white solid. Et₂O, filtered, and precipitated after the addition of ethereal HCl; mp 210-211° C. MS m/z (MH⁺) 479. ¹H NMR 300 MHz (CDCl₃) δ 12.6 (s, 1H), 7.2-7.45 (m, 14H), 3.85 (S, 2H), 3.5-3.1 (m, 10H), 2.6 (d, 1H), 2.5 (d, 2H), 2.05 (m, 2H), 1.2 (br. s, 3H), 1.1 (br. s, 3H). [α]_{D}²⁵=−3.7°.

### EXAMPLE 24

### (+)—N,N-Diethyl-4-[[(1S,5R)-8-phenethyl-8-azabicyclo[3:2:1]oct-3-ylidene]phenylmethyl]benzamide Hydrochloride [1:1]

The mother liquors from the foregoing example were concentrated and partitioned between dilute sodium hydroxide and CH₂Cl₂. The organic solution was concentrated (40.5 g, 0.084 mole) and 32.7 g (0.084 mole) of (−)-ditoluoyl-L-tartaric acid were combined in 500 mL of acetonitrile. The solid was collected and recrystallized twice from acetonitrile. The solid was collected and partitioned between dilute sodium hydroxide and CH₂Cl₂. The organic solution was dried (K₂CO₃) and concentrated. The residue was converted to a hydrochloride salt (Et₂O/HCl) and recrystallized from 2-PrOH to give a white solid; mp 211-212° C. MS m/z (MH⁺) 479. ¹H NMR 300 MHz (CDCl₃) δ 12.6 (s, 1H), 7.2-7.45 (m, 14H), 3.85 (S, 2H), 3.5-3.1 (m, 10H), 2.6 (d, 1H), 2.5 (d, 2H), 2.05 (m, 2H), 1.2 (br. s, 3H), 1.1 (br. s, 3H). [α]_{D}²⁵=+3.7°.

### EXAMPLE 25

### (−)—N,N-Diethyl-4-[[8-phenethyl-8-aza(1R, 5S)bicyclo[3:2:1]oct-3-ylidene]phenylmethyl]thiobenzamide

A mixture of 1.48 g (3.1 mmol) of (−)—N,N-diethyl-4-[[8-phenethyl-8-aza(1R, 5S)bicyclo[3:2:1]oct-3-ylidene]phenylmethyl]benzamide and 1.87 g of Lawesson's reagent was heated at 60° C. in 50 mL of benzene for 2 h. The resulting mixture was flash chromatographed using 5% MeOH in CH₂Cl₂. MS m/z (MH⁺) 495. ¹H NMR 300 MHz (CDCl₃) δ 8.2 (m, 2H), 7.3-7.0 (m, 10H), 6.8 (m, 2H) 4.0 (m, 4H), 3.7-3.2 (m, 10H), 2.7-2.4 (m, 3H), 2.1-1.6 (m, 4H), 1.4 (t, 3H), 1.1 (t, 3H).

### Procedure C

### Ethyl 4-[(8-phenethyl-8-azabicyclo[3.2.1]oct-3-ylidene)phenylmethyl]benzoate

After a mixture of 52 g (0.8 mole) of zinc powder and 800 mL of THF was cooled in an ice bath 44 mL (0.4 mole) of TiCl₄ was added dropwise with stirring. The ice bath was removed and the reaction refluxed for 2 h. A solution of 21.5 g (0.094 mole) of ethyl 4-benzoylbenzoate, 23.9 g (0.094 mole) of 8-phenethyl-8-azabicyclo[3.2.1]octan-3-one, in 100 mL of THF was added dropwise and the reaction was refluxed overnight. After cooling the reaction mixture was poured into a beaker containing K₂CO₃ and ice. Enough K₂CO₃ was added until basic. The solid was filtered off and the organics from the filtrate were separated. The aqueous layer was extracted with Et₂O and the organics were combined, washed with brine and dried over K₂CO₃. The solvent was evaporated in vacuo. The residue was first passed through a flash column, silica gel, (9:1; CH₂Cl₂:MeOH) then a second column using silica gel with 3:1 hexane:acetone to give 21.8 g of the title compound. MS m/z (MH⁺) 452. ¹H NMR (DMSO-d₆) δ 8.0 (d, 2H); 7.35-7.1 (Ar, 12H); 4.3 (t, 2H); 2.8 (m, 2H); 2.7 (m, 2H); 2.4 (bd, 2H); 2.3-2.2 (m, 3H); 1.9 (m, 2H); 1.6 (m, 3H); 1.3 (q, 3H).

### Procedure D

### 4-[(8-Phenethyl-8-azabicyclo[3.2.1]oct-3-ylidene)phenylmethyl]benzoic Acid

A mixture of 22 g (0.048 mole) of ethyl 4-[(8-phenethyl-8-azabicyclo[3.2.1]oct-3-ylidene)phenylmethyl]benzoate, 86 mL of 3N NaOH and 200 mL of EtOH was refluxed for 1 h. After cooling the mixture was made acidic with conc. HCl. The solvent was decanted away from the gum which formed. The gum was titurated with Et₂O and Et₂O/HCl and was placed into a drying oven overnight at 45° C. to yield 19.2 g of 4-[(8-phenethyl-8-azabicyclo[3.2.1]oct-3-ylidene)phenylmethyl]benzoic acid; mp. 285-290° C. MS m/z (MH⁺) 425. ¹H NMR δ 7.9 (d, 2H); 7.4-7.2 (ar, 12H); 3.7 (bs, 2H); 3.0 (bs, 4H); 2.8 (bd, 2H); 2.2 (t, 2H); 2.0 (m, 2H); 1.65 (m, 2H).

### Procedure E

### 4-[(8-Phenethyl-8-azabicyclo[3.2.1]oct-3-ylidene)phenylmethyl]benzoyl Chloride

A mixture of 6 g (0.014 mole) of 4-[(8-phenethyl-8-azabicyclo[3.2.1]oct-3-ylidene)phenylmethyl]benzoic acid, 20 ml of CHCl₂ and 3 mL (0.042 mole) of thionyl chloride were refluxed for 1.5 h. The solvent was evaporated in vacuo to give 6.2 g of 4-[(8-phenethyl-8-azabicyclo[3.2.1]oct-3-ylidene)phenylmethyl]benzoyl chloride. MS m/z (MH⁺) of CH₃OH quench 437.

### EXAMPLE 26

### N-Ethyl-4-[(8-phenethyl-8-azabicyclo[3.2.1]oct-3-ylidene)phenylmethyl]benzamide

A mixture of 11.4 g (0.14 mole) of ethylamine hydrochloride and 150 mL of 3N NaOH and 100 mL of CH₂Cl₂ were cooled in an ice bath. A solution of 4.7 g (0.015 mole) of 4-[(8-phenethyl-8-azabicyclo[3.2.1]oct-3-ylidene)phenylmethyl]benzoyl chloride prepared using Procedure E in 60 mL of CH₂Cl₂ was added. After the addition was complete, the ice bath was removed and the reaction stirred at room temperature for 2 h. The organics were separated off and washed with water, brine and dried (K₂CO₃). The solvent was evaporated in vacuo and converted to the HCl salt with Et₂O/HCl to give 1.86 g of N-ethyl-4-[(8-phenethyl-8-azabicyclo[3.2.1]oct-3-ylidene)phenylmethyl]benzamide; mp 296-298° C. (Decomp). MS m/z (MH⁺) 451. ¹H NMR (DMSO-d₆) δ 8.5 (ar, 1H); 7.8 (d, 2H); 7.4-7.1 (ar, 12H); 4.05 (bs, 2H); 3.4-3.2 (m, 3H); 3.1 (s, 3H); 2.9 (d, 2H); 2.4-2.1 (m, 4H); 1.8 (m, 2H); 1.1 (t, 3H).

### EXAMPLE 27

### (−)-4-[[8-Phenethyl-8-aza(1R,5S)bicyclo[3.2.1]oct-3-ylidene]phenylmethyl]benzamide

4-[[8-Phenethyl-8-aza(1R,5S)bicyclo[3.2.1]oct-3-ylidene]phenylmethyl]benzamide was chromatographed on a CHIRALPAK® Ad™ column eluting with EtOH+0.1% dea. The first enantiomer to elute was collected and converted to the hydrochloride with Et₂O/HCl. [α]_{D}²⁵=−9.7°. MS m/z (MH⁺) 451. ¹H NMR (DMSO-d₆) δ 8.5 (ar, 1H); 7.8 (d, 2H); 7.4-7.1 (ar, 12H); 4.05 (bs, 2H); 3.4-3.2 (m, 3H); 3.1 (s, 3H); 2.9 (d, 2H); 2.4-2.1 (m, 4H); 1.8 (m, 2H); 1.1 (t, 3H).

### EXAMPLE 28

### (+)-4-[[8-Phenethyl-8-aza(1S,5R)bicyclo[3.2.1]oct-3-ylidene]phenylmethyl]benzamide

The second enantiomer to elute was collected and converted to the hydrochloride with Et₂O/HCl. [α]_{D}²⁵=+9.3°. MS m/z (MH⁺) 451. ¹H NMR (DMSO-d₆) δ 8.5 (ar, 1H); 7.8 (d, 2H); 7.4-7.1 (ar, 12H); 4.05 (bs, 2H); 3.4-3.2 (m, 3H); 3.1 (s, 3H); 2.9 (d, 2H); 2.4-2.1 (m, 4H); 1.8 (m, 2H); 1.1 (t, 3H).

### EXAMPLE 29

### 4-[(8-Phenethyl-8-azabicyclo[3.2.1]oct-3-ylidene)phenylmethyl]benzamide Hydrochloride [1:1].

A 1.5 g (0.0034 mole) sample of 4-[(8-phenethyl-8-azabicyclo[3.2.1]oct-3-ylidene)phenylmethyl]benzoyl chloride was cooled in an ice bath. 30 mL of NH₄OH was added dropwise. The ice bath was removed and the mixture was stirred at room temperature for 2 h. The solid was filtered off and dried. The product was passed through a Biotage Flash 40 L (silica gel, 9:1; CH₂Cl₂:MeOH). Conversion to the HCl salt and recrystallization from EtOH/Et₂O gave 0.45 g of 4-[(8-phenethyl-8-azabicyclo[3.2.1]oct-3-ylidene)phenylmethyl]benzamide; mp. 210-212° C. MS m/z (MH⁺) 423. ¹H NMR (DMSO-d₆) δ 7.95 (s, 1H); 7.9 (d, 2H); 7.4-7.2 (ar, 12H); 4.05 (bs, 1H); 3.6 (q, 2H); 2.9 (d, 2H); 2.4-2.1 (m, 5H); 1.8 (m, 3H); 1.1 (t, 3H).

### EXAMPLES 30-47

### N,N-R²,R³-4[(8-Phenethyl-8-azabicyclo[3.2.1]oct-3-ylidene)phenylmethyl]benzamides

By the method of Example 26 and substituting the appropriate amine for ethylamine hydrochloride, the title compound was prepared.

| Ex | Amine | CIMS (MH⁺) |
|---|---|---|
| 30 | morpholine | 493 |
| 31 | diisopropylamine | 506 |
| 32 | bis(methoxyethyl)amine | 538 |
| 33 | pyrrolidine | 477 |
| 34 | cis-2,6-dimethylpiperidine | 519 |
| 35 | N-ethyl-N-(methylallyl)amine | 505 |
| 36 | dipropylamine | 507 |
| 37 | t-butylamine | 479 |
| 38 | 2-fluoroethylamine | 469 |
| 39 | 2-aminothiazole | 507 |
| 40 | 2-methoxyethylamine | 481 |
| 41 | (1H-benzimidazol-2-ylmethyl)amine | 553 |
| 42 | cyclohexylamine | 505 |
| 43 | aniline | 499 |
| 44 | histamine | 517 |
| 45 | cyclopropylamine | 463 |
| 46 | N,N-(dimethylaminopropyl)amine | 508 |
| 47 | N-ethyl-N-(hydroxyethyl)amine | 495 |

### Procedure F

### 8-(2-Benzo[1,3]dioxol-5-ylethyl)-8-azabicyclo[3.2.1]octan-3-one

A 41 g sample of 2,5-dimethoxytetrahydrofuran (0.32 ml) was suspended in 300 mL of H₂O and 40 mL of o-phosphoric acid was added. The mixture was stirred for 3 h then brought to pH 7 by addition of 3N NaOH. Samples of acetone dicarboxylic acid (51 g, 0.15 mol) and (3,4-methylenedioxy) phenethylamine (20 g, 0.12 mol) were added and the mixture stirred at 25° C. for two days. The mixture was made basic by addition of 100 mL of 3N NaOH, was extracted with EtOAc, washed with brine, dried (K₂CO₃) and concentrated. The residue was flash chromatographed using 20% acetone in hexane. The product was a crystalline solid. MS m/z (MH⁺) 274. ¹H NMR 300 MHz (CDCl₃) δ 6.6 (m, 3H), 5.9 (s, 2H), 3.5 (br. m, 2H), 2.85 (s, 4H), 2.65 (dd, 2H), 2.2 (d, 2H), 2.05 (m, 2H), 1.7 (q, 2H).

### Procedure G

### Ethyl [[8-(2-benzo[1,3]dioxol-5-ylethyl)-8-azabicyclo[3:2:1]oct-3-ylidene]phenylmethyl]benzoate

Following the protocol of Procedure C and substituting 8-(2-benzo[1,3]dioxol-5-ylethyl)-8-azabicyclo[3.2.1]octan-3-one for 8-phenethyl-8-azabicyclo[3.2.1]octan-3-one, the title compound was obtained. MS m/z (MH⁺) 496.

### Procedure H

### [[8-(2-Benzo[1,3]dioxol-5-ylethyl)-8-azabicyclo[3:2:1]oct-3-ylidene]phenylmethyl]benzoic Acid

Following the protocol of Procedure D and substituting ethyl-[[8-(2-benzo[1,3]dioxol-5-ylethyl)-8-azabicyclo[3:2: 1]oct-3-ylidene]phenylmethyl]benzoate for ethyl-4-[(8-phenethyl-8-azabicyclo[3.2.1]oct-3-ylidene)phenylmethyl]benzoate, the title compound was obtained. MS m/z (MH⁺) 468.

### Procedure J

### [[8-(2-Benzo[1,3]dioxol-5-ylethyl)-8-azabicyclo[3:2:1]oct-3-ylidene]phenylmethyl]benzoyl Chloride

Following the protocol of Procedure E and substituting [[8-(2-benzo[1,3]dioxol-5-ylethyl)-8-azabicyclo[3:2:1]oct-3-ylidene]phenylmethyl]benzoic acid for 4-[(8-phenethyl-8-azabicyclo[3.2.1]oct-3-ylidene)phenylmethyl]benzoic acid, the title compound was obtained.

### EXAMPLE 48

### N-Ethyl-[[8-(2-Benzo[1,3]dioxol-5-ylethyl)-8-azabicyclo[3:2:1]oct-3-ylidene]phenylmethyl]benzamide

Following the procedure of Example 23 and substituting [[8-(2-benzo[1,3]dioxol-5-ylethyl)-8-azabicyclo[3:2:1]oct-3-ylidene]phenylmethyl]benzoyl chloride for 4-[(8-phenethyl-8-azabicyclo[3.2.1]oct-3-ylidene)phenylmethyl]benzoyl chloride, the title compound was obtained. MS m/z (MH⁺) 495.

### EXAMPLE 49

### N,N-Diethyl-[[8-(2-Benzo[1,3]dioxol-5-ylethyl)-8-azabicyclo[3:2:1]oct-3-ylidene]phenylmethyl]benzamide

Following the procedure of Example 23 and substituting [[8-(2-benzo[1,3]dioxol-5-ylethyl)-8-azabicyclo[3:2:1]oct-3-ylidene]phenylmethyl]benzoyl chloride for 4-[(8-phenethyl-8-azabicyclo[3.2.1]oct-3-ylidene)phenylmethyl]benzoyl chloride and diethyl amine for ethylamine hydrochloride, the title compound was obtained. MS m/z (MH⁺) 523.

### Procedure K

### Ethyl 4-[(8-methyl-8-azabicyclo[3.2.1]oct-3-ylidene)phenylmethyl]benzoate

Following the protocol of Procedure C and substituting tropinone for 8-phenethyl-8-azabicyclo[3.2.1]octan-3-one, the title compound was obtained. MS m/z (MH⁺) 362.

### Procedure L

### 4-[(8-Methyl-8-azabicyclo[3.2.1]oct-3-ylidene)phenylmethyl]benzoic Acid

Following the protocol of Procedure D and substituting ethyl 4-[(8-methyl-8-azabicyclo[3.2.1]oct-3-ylidene)phenylmethyl]benzoate for ethyl 4-[(8-phenethyl-8-azabicyclo[3.2.1]oct-3-ylidene)phenylmethyl]benzoate, the title compound was obtained. MS m/z (MH⁺) 334

### Procedure M

### 4-[(8-Methyl-8-azabicyclo[3.2.1]oct-3-ylidene)phenylmethyl]benzoyl Chloride

Following the protocol of Procedure E and substituting 4-[(8-methyl-8-azabicyclo[3.2.1]oct-3-ylidene)phenylmethyl]benzoic acid for 4-[(8-phenethyl-8-azabicyclo[3.2.1]oct-3-ylidene)phenylmethyl]benzoic acid, the title compound was obtained.

### EXAMPLE 50

### N-Ethyl-4-[(8-Methyl-8-azabicyclo[3.2.1]oct-3-ylidene)phenylmethyl]benzamide

Following the protocol of Example 26 and substituting 4-[(8-methyl-8-azabicyclo[3.2.1]oct-3-ylidene)phenylmethyl]benzoyl chloride for 4-[(8-phenethyl-8-azabicyclo[3.2.1]oct-3-ylidene)phenylmethyl]benzoyl chloride, the title compound was obtained. MS m/z (MH⁺) 361.

### EXAMPLE 51

### N-Ethyl-4-[(8-azabicyclo[3.2.1]oct-3-ylidene)phenylmethyl]benzamide

Following the protocol of Example 2 and substituting N-ethyl-4-[(8-methyl-8-azabicyclo[3.2.1]oct-3-ylidene)phenylmethyl]benzamide for N,N-diethyl-4-[(8-methyl-8-azabicyclo[3.2.1]oct-3-ylidene)phenylmethyl]benzamide, the title compound was obtained. MS m/z (MH⁺) 347.

### EXAMPLE 52

### N-Ethyl-4-[(8-allyl-8-azabicyclo[3.2.1]oct-3-ylidene)phenylmethyl]benzamide

Following the protocol of Example 6 and substituting N-ethyl-4-[(8-azabicyclo[3.2.1]oct-3-ylidene)phenylmethyl]benzamide for N,N-diethyl-4-[(8-azabicyclo[3.2.1]oct-3-ylidene)phenylmethyl]benzamide, the title compound was obtained. MS m/z (MH⁺) 387.

### Procedure N

### 8-[2-(4-Methoxyphenyl)ethyl]-8-azabicyclo[3:2:1]octanone

Following the protocol of Procedure F and substituting (4-methoxy)phenethylamine for (3,4-methylenedioxy)phenethylamine, the title compound was obtained. MS m/z (MH⁺) 260.

### Procedure O

### Ethyl 4-[[8-[2-(4-methoxyphenyl)ethyl]-8-azabicyclo[3:2:1]oct-3-ylidene]phenylmethyl]benzoate

Following the protocol of Procedure C and substituting 8-[2-(4-methoxyphenyl)ethyl]-8-azabicyclo[3:2:1]octanone for 8-phenethyl-8-azabicyclo[3.2.1]octan-3-one, the title compound was obtained. MS m/z (MH⁺) 482.

### Procedure P

### 4-[[8-[2-(4-Methoxyphenyl)ethyl]-8-azabicyclo[3:2:1]oct-3-ylidene]phenylmethyl]benzoic Acid

Following the protocol of Procedure D and substituting ethyl 4-[[8-[2-(4-methoxyphenyl)ethyl]-8-azabicyclo[3:2:1]oct-3-ylidene]phenylmethyl]benzoate for ethyl 4-[(8-phenethyl-8-azabicyclo[3.2.1]oct-3-ylidene)phenylmethyl]benzoate, the title compound was obtained.

### Procedure Q

### 4-[[8-[2-(4-Methoxyphenyl)ethyl]-8-azabicyclo[3:2:1]oct-3-ylidene]phenylmethyl]benzoyl Chloride

Following the protocol of Procedure E and substituting 4-[[8-[2-(4-methoxyphenyl)ethyl]-8-azabicyclo[3:2:1]oct-3-ylidene]phenylmethyl]benzoic acid for 4-[(8-phenethyl-8-azabicyclo[3.2.1]oct-3-ylidene)phenylmethyl]benzoic acid, the title compound was obtained.

### EXAMPLE 53

### -Diethyl-4-[[8-[2-(4-methoxyphenyl)ethyl]-8-azabicyclo[3:2:1]oct-3-ylidene]phenylmethyl]benzamide

Following the protocol of Procedure F and substituting 4-[[8-[2-(4-methoxyphenyl)ethyl]-8-azabicyclo[3:2:1]oct-3-ylidene]phenyl methyl]benzoyl chloride for 4-[(8-phenethyl-8-azabicyclo[3.2.1]oct-3-ylidene)phenylmethyl]benzoyl chloride, the title compound was obtained. MS m/z (MH⁺) 509.

### EXAMPLES 54-63

### -Di-R², R³-4-[[8-[2-(4-methoxyphenyl)ethyl]-8-azabicyclo[3:2:1]oct-3-ylidene]phenylmethyl]benzamides

Using the method of Example 26 and substituting the material from Procedure Q for the material from Procedure E, the following compounds were prepared:

| Ex # | Amine | CIMS (MH⁺) |
|---|---|---|
| 54 | morpholine | 523 |
| 55 | ethylamine | 481 |
| 56 | bis(methoxyethyl)amine | 569 |
| 57 | pyrrolidine | 507 |
| 58 | cis-2,6-dimethylpiperidine | 549 |
| 59 | N-ethyl-(N-methylallyl)amine | 535 |
| 60 | di-n-propylamine | 537 |
| 61 | 2,2,6,6-tetramethylpiperidine | 577 |
| 62 | di-2-propylamine | 537 |

### Procedure R

### N-Ethyl-4-(4-methoxybenzoyl)benzamide

Following the protocol of Procedure A and substituting 4-(4-methoxybenzoyl)benzoic acid for 4-benzoylbenzoic acid and ethylamine hydrochloride for diethylamine, the title compound was obtained. MS m/z (MH⁺) 284.

### EXAMPLE 63

### N-Ethyl-4-[(4-methoxyphenyl)-(8-methyl-8-azabicyclo[3:2:1]oct-3-ylidene)methyl]benzamide

Following the protocol of Example 1 and substituting N-ethyl-4-(4-methoxybenzoyl)benzamide for N,N-diethyl-4-benzoylbenzamide, the title compound was obtained. MS m/z (MH⁺) 391.

### Procedure S

### 2,2,2-Trichloroethyl 3-[(ethylcarbamoylphenyl)-(4-methoxyphenyl)methylene]-8-azabicyclo[3:2:1]octanecarboxylate

A solution of 1.95 g (5.0 mmol) of N-ethyl-4-[(4-methoxyphenyl)-(8-methyl-8-azabicyclo[3:2:1]oct-3-ylidene)methyl]benzamide, 1.03 mL (7.5 mmol) of 2,2,2-trichloroethyl chloroformate and 0.43 mL (2.5 mmol) of diisopropylethylamine was stirred in 50 mL of benzene and 1.38 g (10 mmol) of K₂CO₃ added. The mixture was heated at under reflux for 18 h. Another 0.51 mL of (3.75 mmol) of 2,2,2-trichloroethyl chloroformate and 0.21 mL (1.25 mmol) of diisopropylethylamine was added. The mixture was heated under reflux for 3h. The reaction was cooled and poured into H₂O. The organic layer was washed with dilute HCl and brine, dried (MgSO₄) and concentrated to give 2.09 g of a yellow gum. MS m/z (MH⁺) 553. ¹H NMR 300 MHz (CDCl₃) δ 7.7 (d, 2H), 7.2 (d, 2H), 7.0 (d, 2H), 6.8 (d, 2H), 6.2 br. s, 1H), 4.9 (d, 1H), 4.7 (d, 1H), 4.3 (br. m, 2H), 3.8 (s, 3H), 3.4 (q, 2H), 2.4 (br. m, 4H), 1.9 (m, 2H), 1.7 (m, 2H), 1.2 (t, 3H).

### Procedure T

### 2,2,2-Trichloroethyl 3-[(ethylcarbamoylphenyl)-(4-hydroxyphenyl)methylene]-8-azabicyclo[3:2:1]octanecarboxylate

A solution of 1.03 g (1.82 mmol) of 2,2,2-trichloroethyl 3-[(ethylcarbamoylphenyl)-(4-methoxyphenyl)methylene]-8-azabicyclo[3:2:1]octanecarboxylate in 10 mL of CHCl₃ was cooled to −60° C. under N₂ and 9.1 mL of 1 M BBr₃ in CH₂Cl₂ was added dropwise. The cooling bath was removed and the mixture stirred at 25° C. for 18 h. Saturated aqueous NaHCO₃ was added and the CH₂Cl₂ was evaporated. The solid (1 g) was collected. ¹H NMR 300 MHz (CDCl₃) δ 7.8 (d, 2H), 7.2 (d, 2H), 6.9 (d, 2H), 6.7 (d, 2H), 6.2 (br. s, 1H), 4.9 (d, 1H), 4.7 (d, 1H), 4.4 (br. m, 2H), 3.4 (q, 2H), 2.4 (br. m, 4H), 1.9 (m, 2H), 1.7 (m, 2H), 1.2 (t, 3H).

### EXAMPLE 64

### 4-[(8-Azabicyclo[3:2:1]oct-3-ylidene)-(4-hydroxyphenyl)methyl]-N-ethylbenzamide

A 0.73 g sample (11 mmol) of zinc dust was added to a solution of 0.89 g (1.61 mmol) of 2,2,2-trichloroethyl 3-[(ethylcarbamoylphenyl)-(4-hydroxyphenyl)methylene]-8-azabicyclo[3:2:1]octanecarboxylate in 9 mL of glacial HOAc. The mixture was heated under reflux for 5 h then cooled and the solid removed by filtration and washed with HOAc. The solvent was evaporated and K₂CO₃ was added. The mixture was extracted six times with 20% EtOH in CHCl₃. The solution was dried (Na₂SO₄) and concentrated. The residue was crystallized from EtOH/2-PrOH to give 0.24 g of a white solid. MS m/z (MH⁺) 363. ¹H NMR (DMSO-d₆) δ 8.5 (t, 1H), 7.8 (d, 2H), 7.2 (d, 2H), 6.9 (d, 2H), 6.7 (d, 2H), 3.3 (br. m, 4H), 2.2 (br. m, 4H), 1.5 (m, 4H), 1.1 (t, 3H).

### Procedure U

### N-Diethyl-4-(4-methoxybenzoyl)benzamide

A mixture of 0.75 g (5.5 mmol) of 4-methoxybenzeneboronic acid, 1.5 g (5 mmol) N,N-diethyl-4-iodobenzamide, 0.1 g (0.15 mmol) bistriphenylphosphine palladium(II)dichloride and 2.07 g (15 mmol) of K₂CO₃ in 30 mL of anisole was flushed with carbon monoxide then heated at 80° C. under a CO atmosphere for 5 h. The mixture was filtered and the solvent evaporated. The residue was flash chromatographed 20% acetone in hexane to give the title compound. MS m/z (MH⁺) 312.

### EXAMPLE 65

### N,N-Diethyl-4-[(4-methoxyphenyl)-(8-methyl-8-azabicyclo[3:2:1]oct-3-ylidene)methyl]benzamide

Following the protocol of Example 1 and substituting N,N-diethyl-4-(4-methoxybenzoyl)benzamide for N,N-diethyl-4-benzoylbenzamide, the title compound was obtained. MS m/z (MH⁺) 419.

### Procedure V

### 2,2,2-Trichloroethyl 3-[(diethylcarbamoylphenyl)-(4-methoxyphenyl)methylene]-8-azabicyclo[3:2:1]octanecarboxylate

Following the protocol of Procedure S and substituting N,N-diethyl-4-[(4-methoxyphenyl)-(8-methyl-8-azabicyclo[3:2:1]oct-3-ylidene)methyl]benzamide for N-ethyl-4-[(4-methoxyphenyl)-(8-methyl-8-azabicyclo[3:2:1]oct-3-ylidene)methyl]benzamide, the title compound was obtained.

### Procedure W

### 2,2,2-Trichloroethyl 3-[(diethylcarbamoylphenyl)-(4-hydroxyphenyl)methylene]-8-azabicyclo[3:2:1]octanecarboxylate

Following the protocol of Procedure T and substituting 2,2,2-trichloroethyl 3-[(diethylcarbamoylphenyl)-(4-methoxyphenyl)methylene]-8-azabicyclo[3:2:1]octanecarboxylate for 2,2,2-trichloroethyl 3-[(ethylcarbamoylphenyl)-(4-methoxyphenyl)methylene]-8-azabicyclo[3:2:1]octanecarboxylate, the title compound was obtained.

### EXAMPLE 66

### 4-[(8-Azabicyclo[3:2:1]oct-3-ylidene)-(4-hydroxyphenyl)methyl]-N-diethyl benzamide

Following the protocol for Example 64 and substituting 2,2,2-trichloroethyl 3-[(diethylcarbamoylphenyl)-(4-hydroxyphenyl)methylene]-8-azabicyclo[3:2:1]octanecarboxylate for 2,2,2-trichloroethyl 3-[(ethylcarbamoyl phenyl)-(4-hydroxyphenyl)methylene]-8-azabicyclo[3:2:1]octanecarboxylate, the title compound was obtained. MS m/z (MH⁺) 391.

### EXAMPLE 67

### N,N-Diethyl-4-[(4-methoxyphenyl)-[8-[2-(4-methoxyphenyl)ethyl]-8-azabicyclo[3:2:1]oct-3-ylidene]methyl]benzamide

Following the protocol of Example 1 and substituting 8-[2-(4-methoxyphenyl)ethyl]-8-azabicyclo[3:2:1]octanone for tropinone, the title compound was obtained. MS m/z (MH⁺) 539.

### EXAMPLE 68

### N,N-Diethyl-4-[(4-hydroxyphenyl)-[8-[2-(4-hydroxyphenyl)ethyl]-8-azabicyclo[3:2:1]oct-3-ylidene]methyl]benzamide

Following the protocol of Example 64 and substituting N,N-diethyl-4-[(4-methoxyphenyl)-[8-[2-(4-methoxyphenyl)ethyl]-8-azabicyclo[3:2:1]oct-3-ylidene]methyl]benzamide for 2,2,2-trichloroethyl 3-[(ethylcarbamoylphenyl)-(4-hydroxyphenyl)methylene]-8-azabicyclo[3:2:1]octanecarboxylate, the title compound was obtained. MS m/z (MH⁺) 511.

### EXAMPLE 69

### N-Ethyl-4-[[8-[2-(4-hydroxyphenyl)ethyl]-8-azabicyclo[3:2:1]oct-3-ylidene]phenylmethyl]benzamide

Following the protocol of Procedure T and substituting N-ethyl-4-[[8-[2-(4-methoxyphenyl)ethyl]-8-azabicyclo[3:2:1]oct-3-ylidene]phenylmethyl]benzamide for 2,2,2-trichloroethyl 3-[(ethylcarbamoylphenyl)-(4-methoxy phenyl)methylene]-8-azabicyclo[3:2:1]octanecarboxylate, the title compound was obtained. MS m/z (MH⁺) 467.

### EXAMPLE 70

### N,N-Diethyl-4-[[8-[2-(4-hydroxyphenyl)ethyl]-8-azabicyclo[3:2:1]oct-3-ylidene]phenylmethyl]benzamide

Following the protocol of Procedure T and substituting N,N-diethyl-4-[[8-[2-(4-methoxyphenyl)ethyl]-8-azabicyclo[3:2:1]oct-3-ylidene]phenylmethyl]benzamide for 2,2,2-trichloroethyl 3-[(ethylcarbamoylphenyl)-(4-methoxyphenyl)methylene]-8-azabicyclo[3:2:1]octanecarboxylate, the title compound was obtained. MS m/z (MH⁺) 495.

### EXAMPLE 71

Methyl 4-(bromomethyl) benzoate Compound 71a (22.4 g, 97.8 mmol) was refluxed under N₂ for 6 h in 50 mL of trimethylphosphite. At that time, 100 mL of xylenes was added and the solution was concentrated under vacuum. Coevaporation was repeated until excess trimethylphosphite was completely removed as evidenced by ¹H-NMR. Compound 71 b was obtained in near quantative yield. MS m/z(MH⁺) 259.

Compound 71 b was dissolved in 400 mL of dry THF and cooled to −78° C. LDA (50 mL, 100 mmol) was added dropwise with stirring while the temperature was maintained at less than −70° C. The cooling bath was removed and the resulting solution was allowed to warm to rt. The solution was then cooled to 0° C. and a solution of N-carbethoxy-4-tropinone Compound 71c (19.7 g, 100 mmol) in 200 mL of THF was added over a period of 1 min. Upon complete addition, the cooling bath was removed and the solution was allowed to warm to rt over 22 h. The reaction was quenched by the addition of 500 mL of water followed by 50 mL of brine, and extracted with 300 mL of ethyl ether. The water layer was then extracted with EtOAc (5×200 mL) and the combined organic layers were dried over Na₂SO₄. Evaporation of the solvent gave crude product, which was purified by passage through a plug of flash grade silica gel (0 to 40% EtOAc in hexane) to elute 22 g (68%) of Compound 71d as a white solid. MS m/z(MH⁺) 330.

Compound 71d (22 g, 66.8 mmol) was suspended in 300 mL of dry chloroform and cooled to 0° C. Bromine (6.8 mL, 134 mmol) was added to the mixture over a period of 2 min. After 5 min the cooling bath was removed and the resulting solution was stirred for 18 h at rt. Sodium thiosulfate (10.6 g, 66.8 mmol) was dissolved in a minimum amount of water and added dropwise to the solution until a yellow color was maintained. Toluene (100 mL) was added and the solution was concentrated to a residue. Additional toluene (2×200 mL) was added and the solution was concentrated two more times. The resulting residue was dissolved in 120 mL of MeOH and 180 mL of 3N NaOH was added with stirring. After 3 h at rt, the MeOH was removed under reduced pressure and the pH was adjusted to <2 with concentrated HCl while maintaining the temperature below 15° C. The water layer was then extracted with DCM (5×150 mL) and the combined organic extracts were dried over Na₂SO₄. Evaporation of the solvent gave crude product, which was purified through a plug of flash grade silica gel (0 to 40% EtOAc in hexane). Compound 71e (24 g) was isolated as a white solid and was used without further purification. MS m/z(MH⁺) 394.

Trimethylsilyl iodide (43.3 mL, 304 mmol) was added to a solution of Compound 71e (24 g, 60.9 mmol) in chloroform at rt. The reaction was heated to reflux under N₂ and monitored by LC/MS. The reaction was generally complete after 7 h at which time the solution was cooled to 0° C. and 100 mL of water was carefully added. Sodium bicarbonate (32.2 g, 304 mmol) was added in portions until the pH reached 7-8. Additional sodium bicarbonate (19.4 g, 183 mmol) was added in one portion to the 0° C. solution, followed by 9-fluorenylmethyl succinimidyl carbonate (FmocSu) (30.4 g, 91.4 mmol). The reaction progress was followed by LC/MS and was generally complete after 45 min. At that time 400 mL of water was added. The water layer was extracted with diethyl ether (2×50 mL) and EtOAc (2×50 mL). The combined organic extracts were washed with 0.1 N NaOH (2×50 mL). The aqueous layer was cooled to 0° C., acidified to pH<2 with concentrated HCl, and then extracted with EtOAc (5×200 mL) and the combined organic extracts were dried over Na₂SO₄. Evaporation of the solvent gave crude product, which was purified by flash grade silica gel (20 to 100% DCM in hexane, containing 1% acetic acid) to provide Compound 71f (21 g) as a white solid. The final product contained 5-15% of the vinyl —H compound. MS m/z(MH⁺) 544.

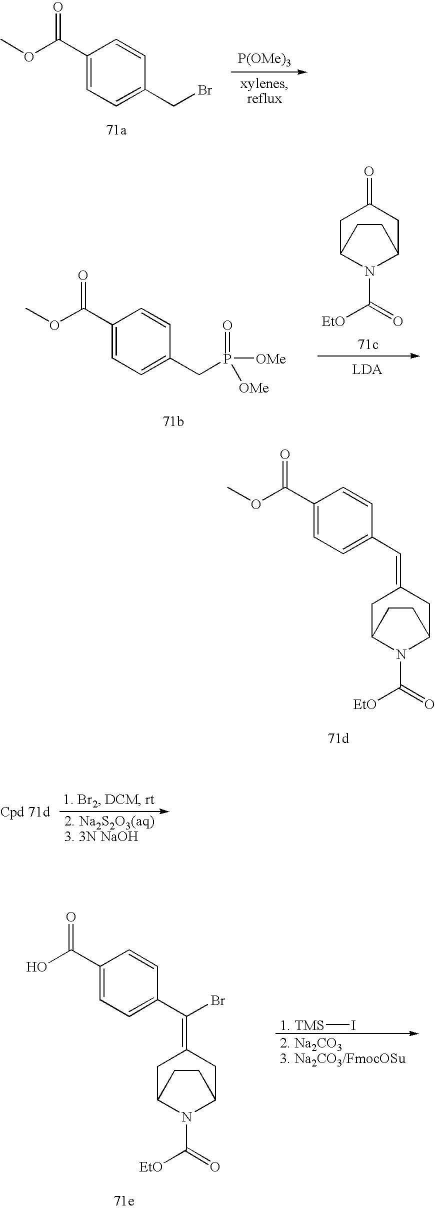

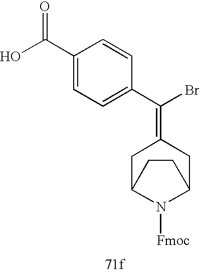

### EXAMPLE 72

FMPB aldehyde resin 72a (26.9 g, 26.9 mmol) [purchased from Irori]was placed in a 3-neck 500 mL round bottom flask equipped with mechanical stirring. The resin was suspended in DCE (100 mL) and to this was added TMOF (25 mL), ethyl amine (67.5 mL, 135 mmol) (2 M in THF), and Na(OAc)₃BH (28.6 g, 135 mmol) were added. The resulting slurry was mixed for 18 h at rt. The resin was filtered and washed with DCM (2×250 mL), MeOH (2×125 mL), water (2×125 mL), MeOH (2×125 mL), DCM (250 mL), MeOH (125 mL), DCM (250 mL), MeOH (125 mL), DCM (4×250 mL). The resulting resin, 72b, was dried under vacuum to constant weight.

To the resin 72b (26.9 mmol), suspended 300 mL DCM, was added Compound 71f (29.2 g, 53.6 mmol) and DIPEA (28 mL, 161 mmol). The resulting slurry was agitated for 1 min. The reaction solution was cooled to 0° C. and 2-chloro-1,3-dimethylimidazolium chloride (13.6 g, 81 mmol) of was added in one portion. The cooling bath was removed and the solution was shaken for 18 h toward rt. The resin was filtered and washed with DCM (2×300 mL). This solution was collected and used for the preparation of Compound 72f as described below. The resulting resin was washed with MeOH (300 mL), DCM (300 mL), MeOH (300 mL), DCM (300 mL), MeOH (300 mL), DCM (3×300 mL). The resulting resin, 72c, was dried under vacuum to constant weight.

For each of the compounds that were prepared from 50 mg of resin 72c; the Fmoc protecting group was removed with 25% piperidine in DMF (2×1 mL) over 30 minutes for each. The resin was filtered and washed with DMF (2×1 mL), MeOH (1 mL), DCM (1 mL), MeOH (1 mL), DCM (1 mL), MeOH (1 mL), DCM (4×1 mL).

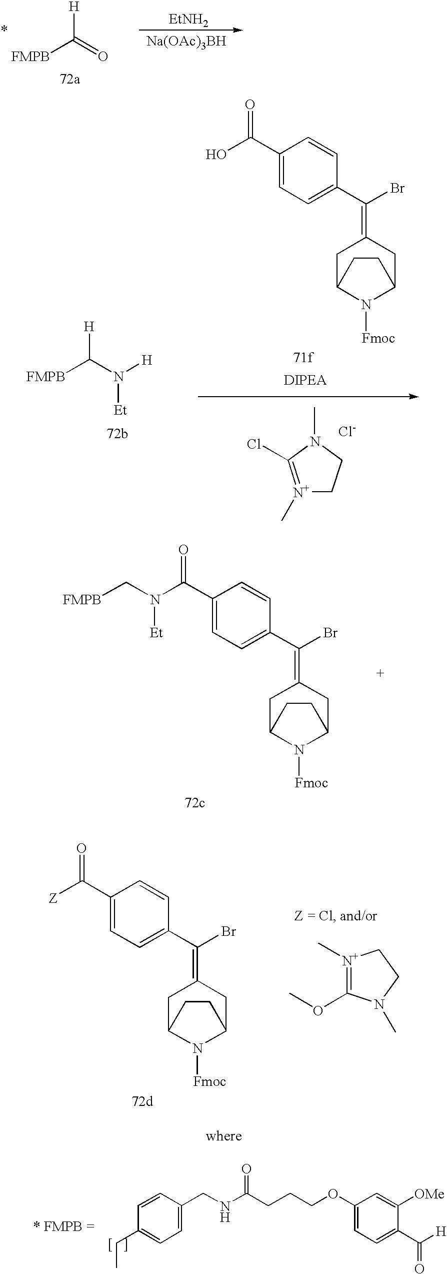

### EXAMPLE 73

### 3{(4-Ethylcarbamoyl-phenyl)-[8-(3-methyl-but-2-enyl)-8-aza-bicyclo[3.2.1]oct-3-ylidene]-methyl}-benzoic acid (Cpd 47)

To a 3 mL teflon™ reaction vessel was added resin 72c (50 mg, 0.025 mmol). The Fmoc protection group was removed and the resin was washed as described above, then washed with DCE (2×1 mL). The resin was suspended in DCE (0.5 mL) and to this was added TMOF (0.5 mL), isovaleraldehyde Compound 73a (0.024 mL, 0.25 mmol), and Na(OAc)₃BH (53 mg, 0.25 mmol). The resulting slurry was agitated for 18 h at rt. The resin was filtered and washed with DCM (2×1 mL), MeOH (2×1 mL), water (2×1 mL), MeOH (2×1 mL), DCM (1 mL), MeOH (1 mL), DCM (1 mL), MeOH (1 mL), DCM (4×1 mL).

Resin 73b was washed with N₂-degassed DMF (2×1 mL) and suspended in DMF (1 mL). To the slurry was added 3-carboxyphenylboronic acid Compound 73c (42 mg, 0.25 mmol), an aqueous solution of K₂CO₃ (35 mg, 0.25 mmol in 75 μl of water), and tetrakis(triphenylphosphine) palladium(0) (15 mg, 0.012 mmol). The resulting slurry was agitated and heated to 80° C. for 18 h. The resin was filtered and washed with DMF (2×1 mL), MeOH (1 mL), DCM (1 mL), MeOH (1 mL), DCM (1 mL), MeOH (1 mL), DCM (4×1 mL) to give resin-bound Compound 73d.

The product was cleaved from the resin using a solution of 1:1 TFA/DCM (1 mL). The cleavage solution was evaporated and the product was purified by semi-preparative reversed phase HPLC on a 20×100 mm J'sphere H-80 YMC column using a gradient of 0.1% TFA/water to 5% water/0.1% TFA/acetonitrile. The eluent was evaporated to yield Compound 47 as a white solid. MS m/z(MH⁺): 459.

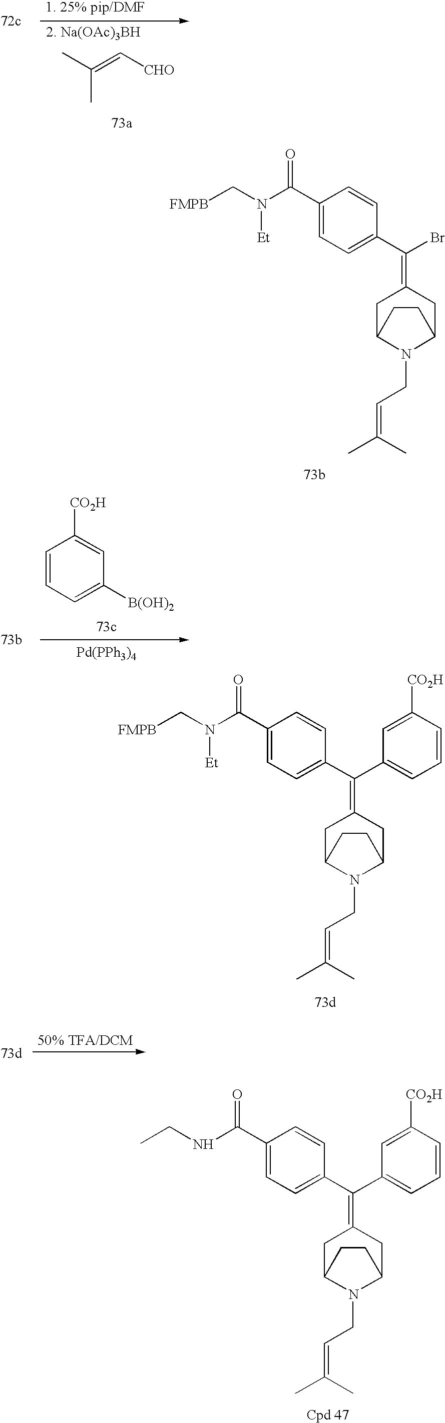

Using the procedure of Example 73 and the appropriate reagents and starting materials known to those skilled in the art, other compounds of the present invention may be prepared, including, but not limited to:

| Cpd | R¹ | R² | M + H⁺ |
|---|---|---|---|
| 3 | quinolin-2-ylmethyl | (3-F)phenyl | 506.3 |
| 4 | quinolin-2-ylmethyl | (4-F)phenyl | 507.3 |
| 5 | (4-acetamido) | benzo[1,3]dioxol- | 538.3 |
| | phenylmethyl | 5-ylmethyl | |
| 6 | 1H-imidazol-2- | benzo[1,3]dioxol- | 471.2 |
| | ylmethyl | 5-ylmethyl | |
| 7 | thiophen-3ylmethyl | benzo[1,3]dioxol- | 487.2 |
| | | 5-ylmethyl | |
| 8 | furan-2-ylmethyl | benzo[1,3]dioxol- | 471.2 |
| | | 5-ylmethyl | |
| 9 | quinolin-2-ylmethyl | benzo[1,3]dioxol- | 532.3 |
| | | 5-ylmethyl | |
| 10 | furan-3-ylmethyl | benzo[1,3]dioxol- | 471.2 |
| | | 5-ylmethyl | |
| 11 | 5-methyl-3H- | benzo[1,3]dioxol- | 485.2 |
| | imidazol-4- | 5-ylmethyl | |
| | ylmethyl | | |
| 12 | 3-Me-thiophen-2-yl | benzo[1,3]dioxol- | 501.2 |
| | | 5-ylmethyl | |
| 13 | quinolin-2-ylmethyl | pyridin-2-yl | 489.3 |
| 14 | (4- | quinolin-3-yl | 545.3 |
| | acetamido)phenyl | | |
| | methyl | | |
| 15 | thiophen-3- | quinolin-3-yl | 494.2 |
| | ylmethyl | | |
| 16 | furan-2-ylmethyl | | 478.2 |
| 17 | furan-3-ylmethyl | quinolin-3-yl | 478.2 |
| 18 | 3-Me-thiophen-2-yl | quinolin-3-yl | 508.3 |
| 19 | quinolin-2-ylmethyl | (2-amino)phenyl | 503.3 |
| 20 | quinolin-2-ylmethyl | (3-CN)phenyl | 513.3 |
| 21 | (4- | Br | 496.2 |
| | acetamido)phenyl | | |
| | methyl | | |
| 22 | 1H-imidazol- | Br | 429.2 |
| | 2ylmethyl | | |
| 23 | thiophen-3- | Br | 445.1 |
| | ylmethyl | | |
| 24 | furan-2-ylmethyl | Br | 429.2 |
| 25 | quinolin-2-ylmethyl | Br | 490.1 |
| 26 | furan-3-ylmethyl | Br | 429.2 |
| 27 | 5-methyl- | Br | 443.2 |
| | 3H-imidazol-4- | | |
| | ylmethyl | | |
| 28 | 3-Me-thiophen-2-yl | Br | 459.1 |
| 29 | quinolin-2-ylmethyl | (3,5- | 516.3 |
| | | dimethyl)phenyl | |
| 30 | quinolin-2-ylmethyl | pyrazin-2-yl | 490.2 |
| 31 | (4- | H | 418.3 |
| | acetamido)phenyl | | |
| | methyl | | |
| 32 | 1H-imidazol-2-yl | H | 351.2 |
| 33 | thiophen-3- | H | 367.2 |
| | ylmethyl | | |
| 34 | furan-2-ylmethyl | H | 351.2 |
| 35 | quinolin-2-ylmethyl | H | 412.3 |
| 36 | furan-3-ylmethyl | H | 351.2 |
| 37 | 5-methyl- | H | 365.2 |
| | 3H-imidazol-4- | | |
| | ylmethyl | | |
| 38 | 3-Me-thiophen-2-yl | H | 381.2 |
| 39 | 1H-imidazol-4- | | 429.2 |
| | ylmethyl | | |
| 40 | thiophen-2- | Br | 445.1 |
| | ylmethyl | | |
| 46 | 1H-imidazol-4- | H | 351.1 |
| | ylmethyl | | |
| 47 | 3-methyl-but-2- | (3-carboxy)phenyl | 459.3 |
| | enyl | | |
| 53 | n-butyl | H | 327.2 |
| 54 | benzo[1,3]dioxol-5- | H | 405.1 |
| | ylmethyl | | |
| 55 | 3-methyl-but-2- | H | 339.2 |
| | enyl | | |
| 56 | pyridin-2-ylmethyl | H | 362.2 |
| 57 | pyridin-3-ylmethyl | H | 362.1 |
| 58 | pyridin-4-ylmethyl | H | 362.2 |
| 59 | 3-phenyl-prop-2- | H | 385.1 |
| | ynyl | | |
| 61 | thiophen-2- | H | 367.1 |
| | ylmethyl | | |
| 62 | phenethyl | H | 375.2 |
| 63 | 3-methyl-but-2- | pyridin-4-yl | 466.3 |
| | enyl | | |
| 64 | thiophen-2- | quinolin-3-yl | 494.3 |
| | ylmethyl | | |
| 65 | benzo[1,3]dioxol-5- | quinolin-3-yl | 532.4 |
| | ylmethyl | | |
| 66 | pyridin-2-ylmethyl | quinolin-3-yl | 489.3 |
| 67 | 3-methyl-but-2- | quinolin-8-yl | 466.3 |
| | enyl | | |
| 68 | thiophen-2- | quinolin-8-yl | 494.3 |
| | ylmethyl | | |
| 69 | benzo[1,3]dioxol-5- | quinolin-8-yl | 532.4 |
| | ylmethyl | | |
| 70 | pyridin-2-ylmethyl | quinolin-8-yl | 489.3 |
| 71 | quinolin-2-ylmethyl | pyridin-3-yl | 489.3 |
| 72 | quinolin-2-ylmethyl | (3-N- | 545.4 |
| | | acetamido)phenyl | |
| 73 | quinolin-2-ylmethyl | (3-acetyl)phenyl | 530.4 |
| 74 | 5-NO₂- | pyridin-3-yl | 489.2 |
| | thiophen-3-yl | | |
| 75 | 5-NO₂- | (3-N- | 545.3 |
| | thiophen-3-yl | acetamido)phenyl | |
| 76 | 5-NO₂- | (3-acetyl)phenyl | 530.3 |
| | thiophen-3-yl | | |
| 77 | 5-Cl-thiophen-2-yl | H | 401.2 |
| 78 | 3-Me- | H | 431.3 |
| | benzothiophen-2-yl | | |
| 87 | 5-carboxy- | (3-carboxy)phenyl | 515.23 |
| | furan-2-yl | | |
| 93 | (3-carboxy)- | phenyl | 481.3 |
| | phenylmethyl | | |
| 94 | (4-carboxy) | phenyl | 481.3 |
| | phenylmethyl | | |
| 95 | 5-carboxy- | phenyl | 471.2 |
| | furan-2-yl | | |

### EXAMPLE 74

### N-Ethyl-4-[(8-fu ran-2-ylmethyl)-8-aza-bicyclo[3.2.1]oct-3-ylidene)-(1H-tetrazol-5-yl)-methyl]benzamide (Cpd 146)

The recovered reaction solution from the formation of resin 72d was cooled to −78° C. and ethyl amine (108.5 mL, 217 mmol, 2 M THF) was added in one portion. The cooling bath was removed and the solution was allowed to stir toward rt for 2 h. The reaction solution was diluted with 100 mL of toluene and concentrated to a residue. The residue was dissolved in 300 mL of THF and 47 mL of thiooctane was added. To the stirred solution was added dropwise 200 μL of DBU and the progress of the Fmoc removal was followed by LC/MS. The reaction was generally complete within 4 h at which time the solvent was removed and the residue triturated with Et₂O. The light brown oil was dried under vacuum and purified by flash grade silica gel. 1% Et₃N/DCM to 19% MeOH/1% Et₃N/DCM was used to elute the product. Evaporation of the eluate provided 16 g of the desired product, Compound 74a, as a white solid. The final product contained 5-15% of the vinyl —H compound as an impurity.

To a nitrogen-degassed solution of vinyl bromide, Compound 74a, (400 mg, 1.1 mmol) in 5 mL of DMF was added zinc cyanide (148 mg, 1.26 mmol) and tetrakis(triphenylphosphine) palladium (0) (132 mg, 0.11 mmol). The solution was heated at 100° C. in a sealed tube under nitrogen for 2 h. The solution was transferred to a separatory funnel with 20 mL of Cl₂Cl₂ and diluted with 15 mL of 1 N NaOH and 50 mL of brine. The layers were separated and the aqueous layer was extracted DCM (3×20 mL) and dried over Na₂SO₄. The solution was filtered and the solvent removed under reduced pressure. The resulting residue was purified by flash silica gel using 1% Et₃N/DCM to 19% MeOH/1% Et₃N/DCM to provide Compound 74b (300 mg) of the desired product. MS m/z(MH⁺): 296

Compound 74b (311 mg, 1.05 mmol) was slurried in 3 mL of toluene and trimethylsilyl azide (564 μL, 4.2 mmol) was added followed by (Bu)₂SnO (52 mg, 0.21 mmol). The resulting solution was heated to 115° C. in a sealed tube for 18 h. Additional trimethylsilyl azide (564 μL, 4.2 mmol) was added followed by (Bu)₂SnO (52 mg, 0.21 mmol) and heated to 115° C. in a sealed tube for 18h The reaction was monitored by LC/MS was generally complete at 24 to 36 h. The reaction was cooled, solubilized in a minimum volume of Cl₂Cl₂, and purified by flash silica gel using 1% Et₃N/DCM to 49% MeOH/1% Et₃N/DCM to provide Compound 74c (220 mg). MS m/z(MH⁺): 339.

To a solution of Compound 74c (10 mg, 0.03 mmol) and 2-furaldehyde Compound 74d (3.7 μL, 0.044 mmol) in DCM (0.3 mL) was added Na(OAc)₃BH (9.4 mg, 0.044 mmol) and AcOH (5 μL). The solution was stirred for 18 h at rt then quenched with 100 μL of water. The solution was concentrated to a residue and purified by semi-preparative reversed phase HPLC on a 20×100 mm J'sphere H-80 YMC column using a gradient of 0.1% TFA/water to 5% water/0.1% TFA/acetonitrile. The solvent was evaporated to yield Compound 146 (4.8 mg) as a white solid. MS m/z(MH⁺): 419.

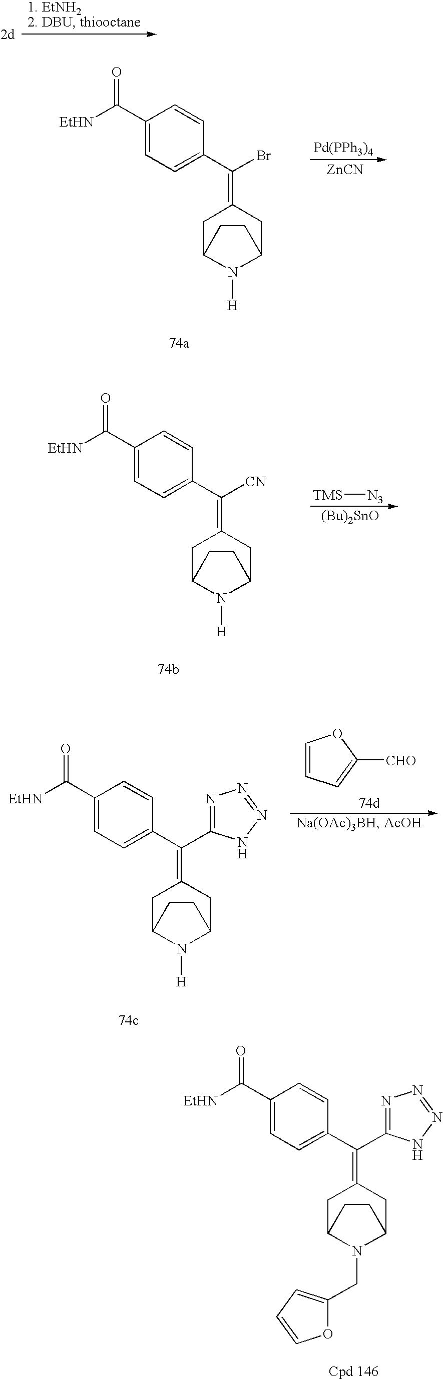

### EXAMPLE 75

### 3-[(4-Ethylcarbamoyl-phenyl)-(8-furan-2-ylmethyl-8-aza-bicyclo[3.2.1]oct-3-ylidene)-methyl]-benzoic acid (Cpd 80)

To a solution of Compound 74a (10 mg, 0.029 mmol) and Compound 74d (8.4 mg, 0.087 mmol) in DCE (0.5 mL) was added Na(OAc)₃BH (12 mg, 0.058 mmol) in DMF (100 μL) and ACOH (5 μL). The reaction mixture was irradiated (μw) at 120° C. for 6 min. After quenching with water, the mixture was concentrated in vacuo. To the resulting residue containing Compound 75a in NMP (0.3 mL) was added K₂CO₃ (12 mg, 0.087 mmol), water (100 μL), 3-carboxyphenylboronic acid Compound 75b (14.4 mg, 0.087 mmol), and tetrakis(triphenylphosphine)palladium (0) (1.5 mg, 0.001 mmol) in NMP (100 μL). The reaction mixture was irradiated (μw) at 180° C. for 10 min. After quenching with water, the mixture was absorbed onto diatomaceous earth and eluted with 5% MeOH/EtOAc. The eluate was concentrated to a residue and purified by reverse-phase chromatography to furnish Compound 80 (11.6 mg, 0.020 mmol) as the TFA salt. MS m/z (MH⁺) 471.

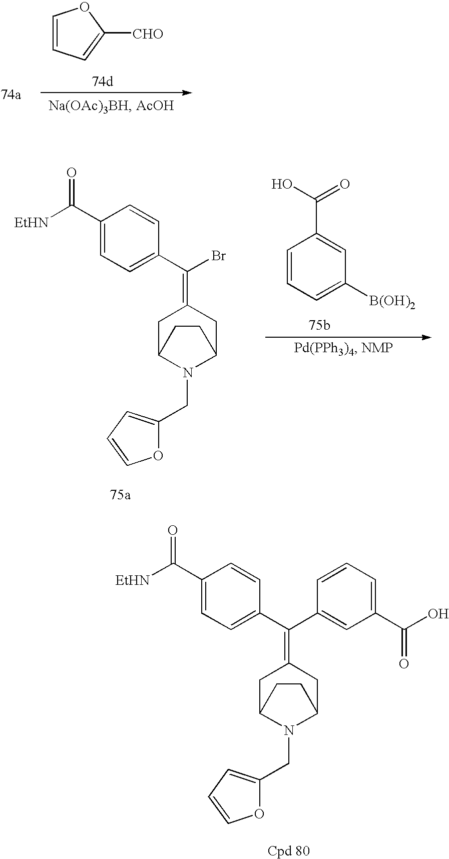

### EXAMPLE 76

### 4-[(8-Aza-bicyclo[3.2.1]oct-3-ylidene)-bromo-methyl]-N,N-diethylbenzamide (Cpd 49)

A portion of the recovered reaction solution from the formation of resin 72d was cooled to −78° C. and diethyl amine (1.6 mL, 15.4 mmol) was added in one portion. The cooling bath was removed and the solution was allowed to stir toward rt for 2 h. The reaction solution was diluted with 10 mL of toluene and concentrated to a residue. The residue was dissolved in 30 mL of THF and 4.7 mL of thiooctane was added. To the stirred solution was added dropwise 2 μL of DBU and the progress of the Fmoc removal was followed by LC/MS. The reaction was generally complete within 4 h at which time the solvent was removed and the residue triturated with Et₂O. The light brown oil was dried under vacuum and purified by flash grade silica gel. 1% Et₃N/DCM to 19% MeOH/1% Et₃N/DCM was used to elute the product. Evaporation of the eluate provided 1.2 g of the desired product, Compound 49, as a white solid. The final product contained 5-15% of the vinyl —H compound as an impurity which was removed by preparative reverse phase HPLC.

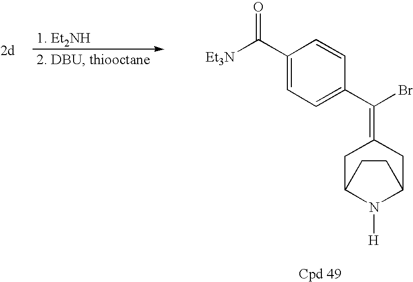

### EXAMPLE 77

### N-ethyl-4{(8-furan-2-yl methyl-8-aza-bicyclo[3.2.1]oct-3-ylidene)-[3-(1H-tetrazol-5-yl)-phenyl]-methyl}-benzamide (Cpd 164)

To a suspension of 3-cyanophenylboronic acid Compound 77a (135 mg, 0.92 mmol), ammonium chloride (148 mg, 2.76 mmol), and DMF (4 mL) was added sodium azide (179 mg, 2.76 mmol). The reaction was irradiated (μw) at 170° C. for 12 min. The reaction mixture was filtered and the filtrate was directly purified by reverse-phase chromatography to furnish Compound 77b (65 mg, 0.34 mmol). MS m/z (MH⁺) 191.

Compound 75a was dissolved in EtOH (0.4 mL) and K₂CO₃ (9 mg, 0.064 mmol) in water (100 μL), Compound 77b (9 mg, 0.047 mmol), and bis(diphenyl-phosphino)ferrocene dichloropalladium (1.8 mg, 0.002 mmol) were added sequentially. The reaction mixture was irradiated (μw) at 150° C. for 10 min. After quenching with water, the mixture was concentrated to a residue, dissolved in DCM (0.3 mL), and absorbed onto diatomaceous earth then eluted with 5% MeOH/EtOAc. The eluate was concentrated to a residue and purified by reverse-phase chromatography to furnish Compound 164 (6.6 mg, 0.009 mmol) as a TFA salt. MS m/z (MH⁺) 495.

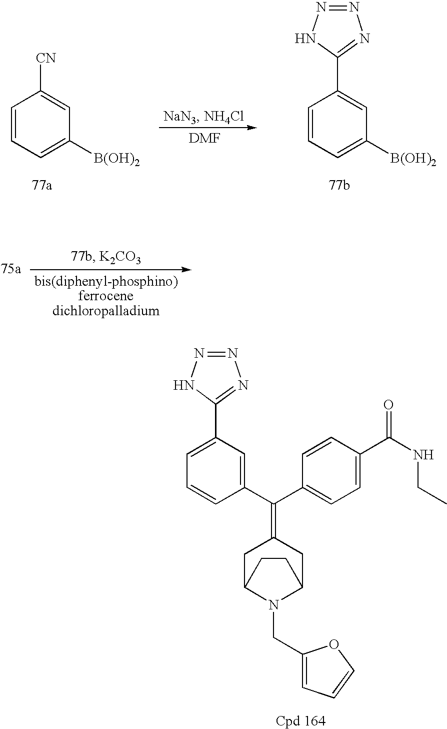

Using the procedure of Example 77 and the appropriate reagents and starting materials known to those skilled in the art, other compounds of the present invention may be prepared, including, but not limited to:

| Cpd | R¹ | R² | R⁵ | R⁶ | M + H⁺ |
|---|---|---|---|---|---|
| 44 | 3-methyl-but-2-enyl | (3-carboxy)phenyl | H | Et | 459.4 |
| 45 | 3-methyl-but-2-enyl | (3-carboxy)phenyl | H | Et | 459.4 |
| 49 | H | Br | Et | Et | 377.2 |
| 80 | furan-2-ylmethyl | (3-carboxy)phenyl | H | Et | 471.4 |
| 81 | furan-3-ylmethyl | (3-carboxy)phenyl | H | Et | 471.4 |
| 82 | pyridin-2-ylmethyl | (3-carboxy)phenyl | H | Et | 482.4 |
| 83 | phenethyl | (3-carboxy)phenyl | H | Et | 495.4 |
| 84 | (4-N-acetamido)phenylmethyl | (3-carboxy)phenyl | H | Et | 538.4 |
| 85 | quinolin-2-ylmethyl | (3-carboxy)phenyl | H | Et | 532.4 |
| 96 | furan-2-ylmethyl | (4-carboxy)phenyl | H | Et | 471.4 |
| 97 | furan-3-ylmethyl | (4-carboxy)phenyl | H | Et | 471.3 |
| 98 | pyridin-2-ylmethyl | (4-carboxy)phenyl | H | Et | 482.3 |
| 99 | phenethyl | (4-carboxy)phenyl | H | Et | 495.5 |
| 100 | quinolin-2-ylmethyl | (4-carboxy)phenyl | H | Et | 532.4 |
| 101 | quinolin-2-ylmethyl | pyrimidin-5-yl | H | Et | 490.3 |
| 102 | thiazol-2-ylmethyl | (3-carboxy)phenyl | H | Et | 488.2 |
| 108 | furan-2-ylmethyl | (3-amino-5-carboxy)phenyl | H | Et | 486.5 |
| 109 | furan-2-ylmethyl | (4-C(O)NEt₂)phenyl | H | Et | 498.5 |
| 110 | furan-3-ylmethyl | (3-amino-5-carboxy)phenyl | H | Et | 486.5 |
| 111 | furan-3-ylmethyl | (4-C(O)NEt₂)phenyl | H | Et | 498.5 |
| 112 | pyridin-2-ylmethyl | (3-amino-5-carboxy)phenyl | H | Et | 497.4 |
| 113 | pyridin-2-ylmethyl | (4-C(O)NEt₂)phenyl | H | Et | 509.4 |
| 114 | 3-methyl-but-2-enyl | (3-amino-5-carboxy)phenyl | H | Et | 474.5 |
| 115 | 3-methyl-but-2-enyl | (4-C(O)NEt₂)phenyl | H | Et | 486.5 |
| 116 | phenethyl | (3-amino-5-carboxy)phenyl | H | Et | 510.5 |
| 117 | phenethyl | (4-C(O)NEt₂)phenyl | H | Et | 522.5 |
| 118 | thiazol-2-ylmethyl | (3-amino-5-carboxy)phenyl | H | Et | 503.4 |
| 119 | thiazol-2-ylmethyl | (4-C(O)NEt₂)phenyl | H | Et | 515.4 |
| 120 | thiophen-2-ylmethyl | (3-amino-5-carboxy)phenyl | H | Et | 502.4 |
| 121 | thiophen-2-ylmethyl | (4-C(O)NEt₂)phenyl | H | Et | 514.5 |
| 122 | thiophen-3-ylmethyl | (4-C(O)NEt₂)phenyl | H | Et | 514.5 |
| 123 | furan-2-ylmethyl | (4-NO₂)phenyl | H | Et | 472.3 |
| 124 | furan-2-ylmethyl | 4-(2-carboxy-2-amino-eth-1-yl)phenyl | H | Et | 514 |
| 125 | furan-2-ylmethyl | 4-(2-carboxy-eth-1-yl)phenyl | H | Et | 499.5 |
| 126 | furan-3-ylmethyl | (4-NO₂)phenyl | H | Et | 472.2 |
| 127 | furan-3-ylmethyl | 4-(2-carboxy-eth-1-ylphenyl | H | Et | 499.2 |
| 128 | thiophen-3-ylmethyl | (4-NO₂)phenyl | H | Et | 488.1 |
| 129 | thiophen-3-ylmethyl | 4-(2-carboxy-eth-1-yl)phenyl | H | Et | 515.3 |
| 130 | thiazol-2-ylmethyl | (4-NO2)phenyl | H | Et | 489.2 |
| 131 | thiazol-2-ylmethyl | 4-(2-carboxy-2-amino-eth-1-yl)phenyl | H | Et | 531.1 |
| 132 | thiazol-2-ylmethyl | 4-(2-carboxy-eth-1-yl)phenyl | H | Et | 516.2 |
| 133 | thiazol-2-ylmethyl | H | H | Et | 368.2 |
| 134 | phenethyl | (4-NO₂)phenyl | H | Et | 496.2 |
| 135 | phenethyl | 4-(2-carboxy-eth-1-yl)phenyl | H | Et | 523.3 |
| 136 | 3-methyl-but-2-enyl | (4-NO2)phenyl | H | Et | 460.2 |
| 137 | 3-methyl-but-2-enyl | 4-(2-carboxy-eth-1-yl)phenyl | H | Et | 487.3 |
| 138 | furan-3-ylmethyl | (4-SO₂NH₂)phenyl | H | Et | 506.1 |
| 139 | thiophen-3-ylmethyl | (4-SO₂NH₂)phenyl | H | Et | 522.3 |
| 140 | thiazol-2-ylmethyl | (4-SO₂NH₂)phenyl | H | Et | 523.2 |
| 141 | thiophen-2-ylmethyl | (4-SO₂NH₂)phenyl | H | Et | 522.3 |
| 142 | 3-methyl-but-2-enyl | (4-SO₂NH₂)phenyl | H | Et | 494.2 |
| 143 | furan-3-ylmethyl | CN | H | Et | 376.6 |
| 144 | furan-3-ylmethyl | 1H-tetrazol-5-yl | H | Et | 419.3 |
| 145 | H | CN | H | Et | 296.3 |
| 146 | furan-2-ylmethyl | 1H-tetrazol-5-yl | H | Et | 419.1 |
| 147 | 3-methyl-but-2-enyl | 1H-tetrazol-5-yl | H | Et | 407.1 |
| 148 | thiophen-3-ylmethyl | 1H-tetrazol-5-yl | H | Et | 435.3 |
| 149 | phenethyl | 1H-tetrazol-5-yl | H | Et | 443.5 |
| 150 | thiazol-2-ylmethyl | 1H-tetrazol-5-yl | H | Et | 436.3 |
| 151 | H | 1H-tetrazol-5-yl | H | Et | 339.5 |
| 152 | furan-3-ylmethyl | (3-carboxy-5-NO₂)phenyl | H | Et | 516.4 |
| 153 | furan-3-ylmethyl | (3-aminomethyl)phenyl | H | Et | 456.5 |
| 154 | pyridin-2-ylmethyl | (3-carboxy-5-NO₂)phenyl | H | Et | 527.4 |
| 155 | 3-methyl-but-2-enyl | (3-carboxy-5-NO₂)phenyl | H | Et | 504.5 |
| 156 | 3-methyl-but-2-enyl | (3-aminomethyl)phenyl | H | Et | 444.5 |
| 157 | phenethyl | (3-carboxy-5-NO₂)phenyl | H | Et | 540.5 |
| 158 | phenethyl | (3-aminomethyl)phenyl | H | Et | 480.5 |
| 159 | thiazol-2-ylmethyl | (3-carboxy-5-NO₂)phenyl | H | Et | 533.4 |
| 160 | thiazol-2-ylmethyl | (3-aminomethyl)phenyl | H | Et | 473.4 |
| 161 | thiophen-3-ylmethyl | (3-carboxy-5-NO₂)phenyl | H | Et | 532.3 |
| 162 | thiophen-3-ylmethyl | (3-aminomethyl)phenyl | H | Et | 472.4 |
| 163 | 3-methyl-but-2-enyl | 3-(1H-tetrazol-4-yl)phenyl | H | Et | 483.5 |
| 164 | furan-2-ylmethyl | 3-(1H-tetrazol-4-yl)phenyl | H | Et | 495.5 |
| 165 | pyridin-2-ylmethyl | 3-(1H-tetrazol-4-yl)phenyl | H | Et | 506.5 |
| 166 | phenethyl | 3-(1H-tetrazol-4-yl)phenyl | H | Et | 519.5 |
| 167 | thiazol-2-ylmethyl | 3-(1H-tetrazol-4-yl)phenyl | H | Et | 512.4 |
| 168 | thiophen-2-ylmethyl | 3-(1H-tetrazol-4-yl)phenyl | H | Et | 511.4 |
| 169 | thiophen-3-ylmethyl | 3-(1H-tetrazol-4-yl)phenyl | H | Et | 511.4 |
| 170 | furan-3-ylmethyl | 3-(1H-tetrazol-4-yl)phenyl | H | Et | 495.5 |
| 174 | thiazol-2-ylmethyl | (4-carboxy)phenyl | H | Et | 488.5 |
| 175 | thiophen-3-ylmethyl | (3-carboxy)phenyl | H | Et | 487.4 |
| 176 | thiophen-3-ylmethyl | (4-carboxy)phenyl | H | Et | 487.4 |
| 177 | furan-3-ylmethyl | (4-C(═O)NH₂)phenyl | H | Et | 470.6 |
| 178 | furan-3-ylmethyl | (3-hydroxymethyl)phenyl | H | Et | 457.5 |
| 179 | furan-2-ylmethyl | (3-hydroxymethyl)phenyl | H | Et | 457.3 |
| 180 | furan-2-ylmethyl | (4-C(═O)NH₂)phenyl | H | Et | 470.4 |
| 181 | pyridin-2-ylmethyl | (3-hydroxymethyl)phenyl | H | Et | 468.4 |
| 182 | pyridin-2-ylmethyl | (4-NHSO₂Me)phenyl | H | Et | 531.4 |
| 183 | pyridin-2-ylmethyl | (4-C(═O)NH₂phenyl | H | Et | 481.3 |
| 184 | phenethyl | (3-hydroxymethyl)phenyl | H | Et | 482.4 |
| 185 | phenethyl | (4-NHSO₂Me)phenyl | H | Et | 544.3 |
| 186 | thiazol-2-ylmethyl | (3-hydroxymethyl)phenyl | H | Et | 474.3 |
| 187 | thiazol-2-ylmethyl | (4-NHSO₂Me)phenyl | H | Et | 537.2 |
| 188 | thiazol-2-ylmethyl | (4-C(═O)NH₂phenyl | H | Et | 487.2 |
| 189 | thiophen-3-ylmethyl | (3-hydroxymethyl)phenyl | H | Et | 473.3 |
| 190 | thiophen-3-ylmethyl | (4-NHSO₂Me)phenyl | H | Et | 536.3 |
| 191 | thiophen-3-ylmethyl | (4-C(═O)NH₂)phenyl | H | Et | 486.3 |
| 192 | furan-2-ylmethyl | (4-hydroxymethyl)phenyl | H | Et | 457.3 |
| 193 | pyridin-2-ylmethyl | (4-hydroxymethyl)phenyl | H | Et | 468.3 |
| 194 | 3-methyl-but-2-enyl | (4-hydroxymethyl)phenyl | H | Et | 445.2 |
| 195 | thiazol-2-ylmethyl | (4-hydroxymethyl)phenyl | H | Et | 474.2 |
| 196 | thiophen-3-ylmethyl | (4-hydroxymethyl)phenyl | H | Et | 473.2 |
| 201 | H | 1H-tetrazol-5-yl | H | Et | 339.1 |
| 202 | H | 1H-tetrazol-5-yl | H | Et | 339.1 |
| 203 | furan-2-ylmethyl | 1H-tetrazol-5-yl | H | Et | 419.1 |
| 204 | furan-2-ylmethyl | 1H-tetrazol-5-yl | H | Et | 419.1 |
| 209 | furan-3-ylmethyl | CN | H | Et | 376.4 |
| 210 | furan-3-ylmethyl | CN | H | Et | 376.4 |
| 211 | furan-3-ylmethyl | Br | H | Et | 429.3 |
| 212 | furan-3-ylmethyl | Br | H | Et | 429.3 |
| 215 | thiophen-3-ylmethyl | 3-(1H-tetrazol-4-yl)phenyl | H | Et | 511.4 |
| 216 | thiophen-3-ylmethyl | 3-(1H-tetrazol-4-yl)phenyl | H | Et | 511.4 |
| 219 | pyridin-2-ylmethyl | Br | H | Et | 440.4 |
| 220 | pyridin-2-ylmethyl | Br | H | Et | 440.4 |
| 221 | thiophen-3-ylmethyl | Br | H | Et | 445.3 |
| 222 | thiophen-3-ylmethyl | Br | H | Et | 445.3 |
| 223 | pyridin-2-ylmethyl | (4-C(O)NEt₂)phenyl | H | Et | 509.5 |
| 224 | pyridin-2-ylmethyl | (4-C(O)NEt₂)phenyl | H | Et | 509.5 |
| 225 | 3-methyl-but-2-enyl | Br | H | Et | 417.3 |
| 226 | 3-methyl-but-2-enyl | Br | H | Et | 417.3 |

### EXAMPLE 78

### 4-[(8-Cyanomethyl-8-aza-bicyclo[3.2.1]oct-3-ylidene)-phenyl-methyl]-N-ethyl-benzamide (Cpd 43)

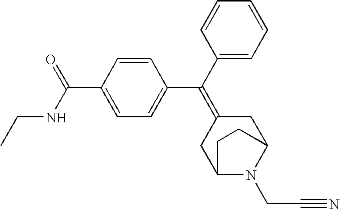

Following the procedure of Example 5, substituting 4-[(8-aza-bicyclo[3.2.1]oct-3-ylidene)-phenyl-methyl]-N-ethyl-benzamide for N,N-diethyl-4-[(8-azabicyclo[3.2.1]oct-3-ylidene)phenylmethyl]benzamide and iodoacetonitrile for allyl bromide, the title compound was obtained. MS m/z (MH⁺) 386.

### EXAMPLE 79

### 4-[8-(3-Cyano-3,3-diphenylpropyl)-8-aza-bicyclo[3.2.1]oct-3-ylidenemethyl]-N-ethyl-benzamide (Cpd 106)

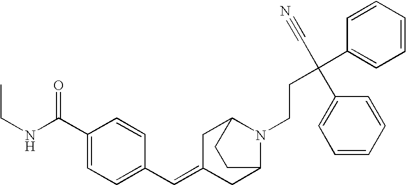

Following the procedure of Example 5, substituting 4-(8-Aza-bicyclo[3.2.1]oct-3-ylidenemethyl)-N-methyl-benzamide for N,N-diethyl-4-[(8-azabicyclo[3.2.1]oct-3-ylidene)phenylmethyl]benzamides and 4-bromo-2,2-diphenyl-butyronitrile for allyl bromide, the title compound was prepared. MS m/z (MH⁺) 290. ¹H NMR 300 MHz (CDCl₃) δ 7.7 (d, 1H); 7.6 (m, 14H); 6.1 (s, 1H); 3.5 (q, 2H); 3.3 (m, 2H); 2.9 (m, 2H); 2.7 (d, 1H); 2.35 (d, 2H) 2.2-1.9 (m, 3H); 1.5 (m, 4H); 1.2 (t, 3H).

### EXAMPLE 80

### 4-[8-(3-Dimethylcarbamoyl-3,3-diphenyl-propyl)-8-aza-bicyclo[3.2.1]oct-3-ylidenemethyl]-N-ethyl-benzamide (Cpd 107)

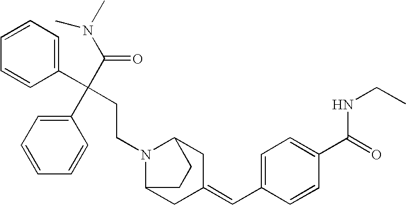

A sample of 4-(8-aza-bicyclo[3.2.1]oct-3-ylidenemethyl)-N-ethyl-benzamide (0.68 g, 0.0025 mol) was mixed with 0.8 g (0.0026 mol) of 3,3-diphenyl-dihydro-furan-2-ylideneamine bromide, Na₂CO₃ (0.26 g, 0.0026 mol) and 5 mL of MEK and the mixture was heated to 60° C. overnight. After cooling, the reaction was diluted with Et₂O and water. The organic phase was washed sequentially with water and brine, then dried (K₂CO₃). The solvent was evaporated in vacuo and the residue was passed through a silica gel column (9:1 CH₂Cl₂:MeOH) to give 10 mg of 4-[8-(3-Dimethylcarbamoyl-3,3-diphenyl-propyl)-8-aza-bicyclo[3.2.1]oct-3-ylidenemethyl]-N-ethyl-benzamide. mp 137-139° C.; MS m/z (MH⁺) 536.1.

### EXAMPLE 81

### N-Ethyl-4-{phenyl-[8-(1H-tetrazol-5-ylmethyl)-8-aza-bicyclo[3.2.1]oct-3-ylidene]-methyl}-benzamide (Cpd 90)

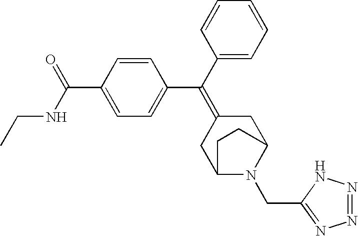

A solution of 0.85 mL (1.7 mmol) of trimethylaluminium 2.0 M in toluene was cooled to below 5° C. and 0.23 mL (1.7 mmol) of TMS azide was added dropwise. After stirring for 15 min, a solution of 4-[(8-cyanomethyl-8-aza-bicyclo[3.2.1]oct-3-ylidene)-phenyl-methyl]-N-ethyl-benzamide (0.32 g, 0.8 mmol) in 2.5 mL of CH₂Cl₂ was added dropwise and the reaction was heated to 80° C. for 18 h. The reaction was cooled and transferred via syringe into 2 mL of 6 N HCl and 2 mL of EtOAc. The liquid was decanted from the gum, and the gum was triturated with Et₂O to give 0.108 g of N-ethyl-4-{phenyl-[8-(1H-tetrazol-5-yl methyl)-8-aza-bicyclo[3.2.1]oct-3-ylidene]-methyl}-benzamide; MS m/z (MH⁺) 429; ¹H NMR 300 MHz (DMSO-d₆) δ 8.5 (ar, 1H); 7.8 (d, 2H); 7.4-7.1 (m, 7H); 3.4 (t, 2H); 3.2 (q, 2H); 2.8 (m, 2H); 2.2-2.1 (m, 5H); 1.9-1.6 (m, 2H); 1.0 (t, 2H).

### EXAMPLE 82

### N-Ethyl-4-[phenyl-(8-quinolin-2-ylmethyl-8-aza-bicyclo[3.2.1]oct-3-ylidene)-methyl]-benzamide (Cpd 79)

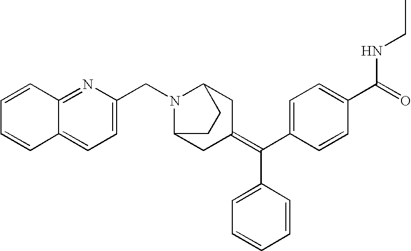

Following the procedure of Example 18, substituting 4-[(8-aza-bicyclo[3.2.1]oct-3-ylidene)-phenyl-methyl]-N-ethyl-benzamide for N,N-diethyl-4-[(8-azabicyclo[3.2.1]oct-3-ylidene)phenylmethyl]benzamides and 2-quinolinecarboxaldehyde for propionaldehyde, the target compound was prepared. MS m/z (MH⁺) 488; ¹H NMR 300 MHz (DMSO-d₆) δ 8.5 (m, 1H); 8.1 (m, 2H); 7.9-7.6 (m, 5H); 7.4-7.1 (m, 7H); 4.1 (s, 2H); 3.2 (m, 2H); 2.9 (d, 2H); 2.4-2.2 (m, 6H); 1.9 (m, 2H); 1.1 (t, 3H).

### EXAMPLE 83

### 4-(8-Aza-bicyclo[3.2.1]oct-3-ylidenemethyl)-N,N-diethylbenzamide (Cpd 48)

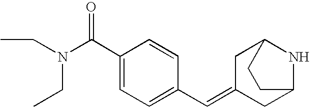

Using the methods described in Procedures D, E, and F, Compound 71d was converted to its corresponding amide by reaction with diethylamine. This intermediate was subsequently deprotected with trimethylsilyl iodide using the method described in Example 71 to afford the title compound. MS m/z (MH⁺) 387(M+41); ¹H NMR 300 MHz (DMSO-d₆) δ 7.3 (m, 4H); 6.5 (s,1H); 4.0 (d, 2H); 3.5-3.1 (m, 4H); 2.9-2.3 (m, 4H); 2.0-1.6 (m, 4H); 1.0 (bs, 6H).

By the protocol of Example 83 using the appropiate amine the following compounds were prepared;

| amine | Cpd |
|---|---|
| ethylamine | 4-(8-Aza-bicyclo[3.2.1]oct-3- |
| | ylidenemethyl)-N-ethyl-benzamide |
| methylamine | 4-(8-Aza-bicyclo[3.2.1]oct-3- |
| | ylidenemethyl)-N-methyl-benzamide |

### EXAMPLE 84

### N,N-R₂-ethyl-4-(8-R¹-8-aza-bicyclo[3.2.1]oct-3-ylidenemethyl)-benzamides

Using the protocol of Example 18, substituting 4-(8-Aza-bicyclo[3.2.1]oct-3-ylidenemethyl)-N,N-R₄R₅-benzamide for N,N-diethyl-4-[(8-azabicyclo[3.2.1]oct-3-ylidene)phenylmethyl]benzamides, the following target compounds were prepared from their corresponding aldehydes.

| JNJ | Cpd | R¹ | R² | R⁵ | R⁶ | M + H⁺ |
|---|---|---|---|---|---|---|
| 10212943 | 2 | 1-benzyl-1- | H | H | Et | 505.3 |
| | | (t-butoxycarbonyl | | | | |
| | | amino)ethyl | | | | |
| 10318646 | 50 | phenethyl | H | H | Me | 362.1 |
| 10318750 | 51 | phenethyl | H | Et | Et | 404.2 |
| 10319920 | 52 | thien-3-ylmethyl | H | Et | Et | 396.1 |
| 10320453 | 60 | pyridin-2-ylmethyl | H | H | Me | 349.1 |
| 10329761 | 86 | (2-OH)phenethyl | H | H | Et | 391.9 |
| 17062669 | 104 | methyl | H | H | Me | 313.1 |

### EXAMPLE 85

### 4-[8-(2-Amino-3-phenyl-propyl)-8-aza-bicyclo[3.2.1]oct-3-ylidenemethyl]-N-ethyl-benzamide Hydrochloride (Cpd 1)

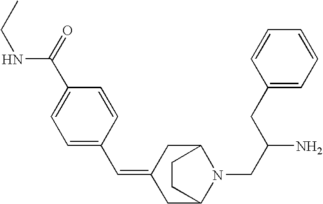

A solution of 0.7 g of {1-benzyl-2-[3-(4-ethylcarbamoyl-benzylidene)-8-aza-bicyclo[3.2.1]oct-8-yl]-ethyl}-carbamic acid t-butyl ester was stirred with 8 mL of TFA and 1 mL of water overnight. The solvent was evaporated in vacuo and the residue was passed through a silica gel column (9:1 CH₂Cl₂:MeOH). The product was treated with Et₂O/HCl in i-Pr to give 4-[8-(2-amino-3-phenyl-propyl)-8-aza-bicyclo[3.2.1]oct-3-ylidenemethyl]-N-ethyl-benzamide hydrochloride. MS m/z (MH⁺) 404.

### EXAMPLE 86

### 4-[(8{2-[4-(4-Chloro-benzoyl)-1-methyl-1H-pyrrol-2-yl]-2-oxo-ethyl}-8-azabicyclo[3.2.1]oct-3-ylidene)-phenyl-methyl]-N-ethyl-benzamide (Cpd 88)

A 0.25 g (0.72 mmol) sample of N-ethyl-4-[(8-azabicyclo[3.2.1]oct-3-ylidene)phenylmethyl]-benzamide (Example 51), 0.28 g of chloro-1-[4-(4-chlorobenzoyl)-1-methyl-1H-pyrrol-2-yl]-ethanone, 0.18 mL of diisopropylethylamine and 10 mL of EtOH were refluxed for 4 h then stirred overnight under argon at room temperature. After evaporating the solvent in vacuo the residue was chromatographed on silica gel (9:1 CH₂Cl₂:MeOH). Treatment of the product with Et₂O/HCl gave 0.18 g of 4-[(8-{2-[4-(4-chloro-benzoyl)-1-methyl-1H-pyrrol-2-yl]-2-oxo-ethyl}-8-azabicyclo[3.2.1]oct-3-ylidene)-phenyl-methyl]-N-ethyl-benzamide. Mp 195-196° C.; MS m/z (MH⁺) 606; ¹H NMR 300 MHz (DMSO-d₆) δ 8.5 (m, 1H); 7.9-7.6 (m, 7H); 7.4-71. (m, 7H); 4.7 (d, 1H); 4.1(bs, 2H); 4.05 (s, 3H); 3.5-3.2 (m, t, 4H); 2.9 (d, 2H); 2.5-2.2 (m, 3H); 1.9 (m, 2H); 1.1(t, 3H).

### EXAMPLE 87

### 4-(8-Aza-bicyclo[3.2.1]oct-3-ylidenemethyl)-N-methyl-benzamide (Cpd 103)

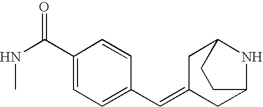

Compound 71 d was treated with N-methylamine using the methods described in Procedures D, E, and F. The resulting product was treated with TMSI as described in Example 71 to yield the title compound. Mp 164-166° C.; MS m/z (MH⁺) 257.1; ¹H NMR 300 MHz (DMSO-d₆) δ 8.3 (d, 1H); 7.8 (d, 2H); 7.3 (d, 2H); 6.5 (s, 1H); 4.0 (bd, 2H); 2.7 (s, 3H); 2.9-2.3 (m, 5H); 2.4 (s, 3H); 1.9-1.7 (m, 3H); 1.5 (m, 1H)

### Procedure X

### 4-[(8-Aza-bicyclo[3.2.1]oct-3-ylidene)-pyridin-3-yl-methyl]-N-ethyl-benzamide

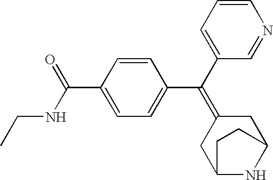

Compound 71e (Example 71) was converted to 3-[bromo-(4-ethylcarbamoyl-phenyl)-methylene]-8-aza-bicyclo[3.2.1]octane-8-carboxylic acid ethyl ester by standard procedures previously described in Procedure E and Example 26.

An 11.3 g (0.027 mol) sample of 3-[bromo-(4-ethylcarbamoyl-phenyl)-methylene]-8-aza-bicyclo[3.2.1]octane-8-carboxylic acid ethyl ester and 1.2 g (4 mol %) of tetrakis triphenylphosphine palladium(0) in 180 mL of DME was stirred for 30 min. A 5 g (0.027 mol) sample of pyridine-3-boronic acid and 36 mL of NaHCO₃ (aq) was added and the reaction was refluxed overnight. After cooling the reaction was partitioned between CH₂Cl₂ and water, the organic phase was separated, washed sequentially with water and brine, then dried (Na₂SO₄). The solvent was removed in vacuo and the residue was chromatographed through silica gel (90:10:1 CH₂Cl₂:MeOH:NH₄OH). The sample was passed a second time through silica gel (9:1 CH₂Cl₂:MeOH). The resulting residue was treated with Et₂O/HCl to give 9.5 g of 3-[(4-ethylcarbamoyl-phenyl)-pyridin-3-yl-methylene]-8-aza-bicyclo[3.2.1]octane-8-carboxylic acid ethyl ester. MS m/z (MH⁺) 421.

Using standard procedures as previously described in Example 71, 3-[(4-ethylcarbamoyl-phenyl)-pyridin-3-yl-methylene]-8-aza-bicyclo[3.2.1]octane-8-carboxylic acid ethyl ester was treated with TMSI to give 4-[(8-aza-bicyclo[3.2.1]oct-3-ylidene)-pyridin-3-yl-methyl]-N-ethyl-benzamide. MS m/z (MH⁺) 347.

### EXAMPLE 89

### N-Ethyl-4-{pyridin-3-yl-[8-(thiophene-2-carbonyl)-8-aza-bicyclo[3.2.1]oct-3-ylidene]-methyl}-benzamide (Cpd 208)

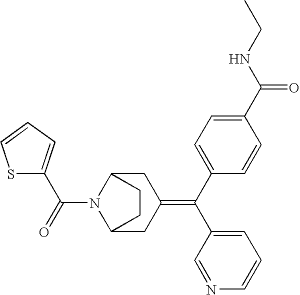

A 0.2 g (0.58 mmol) sample of 4-[(8-aza-bicyclo[3.2.1]oct-3-ylidene)-pyridin-3-yl-methyl]-N-ethyl-benzamide was treated with 0.1 g (0.68 mmol) of thiophene-2-carbonyl chloride and 0.1 g (1.7 mmol) of K₂CO₃ in 5 mL of DMF and the reaction was refluxed for 1 h. After 1 h, the mixture was stirred overnight under argon at room temperature. Upon cooling, NaHCO₃ (aq) was added and the reaction was extracted with Et₂O. The organic phase was washed sequentially with water and brine, then dried (K₂CO₃). After removing the solvent in vacuo the product was chromatographed on silica gel (90:10:1 CH₂Cl₂:MeOH:NH₄OH) to give 6.4 mg of N-ethyl-4-{pyridin-3-yl-[8-(thiophene-2-carbonyl)-8-aza-bicyclo[3.2.1]oct-3-ylidene]-methyl}-benzamide. MS m/z (MH⁺) 458.3; ¹H NMR 300 MHz (CDCl₃) δ 8.4 (m, 2H); 7.8 (m, 2H); 7.4 (m, 3H); 7.3-7.1 (m, 4H); 7.0 (m, 1H); 3.4 (a, 2H); 2.6-2.2 (m, 6H); 2.0 (m, 2H); 1.9 (m, 2H); 1.1 (t, 3H).

### EXAMPLE 90

Using the protocol of Example 89, substituting the appropriate acid chloride for thiophene-2-carbonyl chloride, the following compounds of the present invention were prepared.

| JNJ | Cpd | R¹ | R² | R⁵ | R⁶ | M + H⁺ |
|---|---|---|---|---|---|---|
| 19377501 | 213 | pyridin-2- | pyridin-3-yl | H | Et | 453.3 |
| | | ylcarbonyl | | | | |
| 19385938 | 214 | furan-3- | pyridin-3-yl | H | Et | 442.6 |
| | | ylcarbonyl | | | | |

### EXAMPLE 91

### N-Ethyl-4-[(8-formyl-8-aza-bicyclo[3.2.1]oct-3-ylidene)-pyridin-3-yl-methyl]-benzamide (Cpd 207)

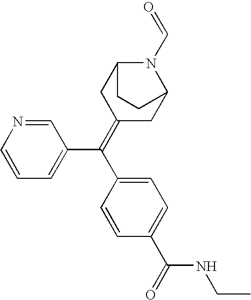

A 4 mL sample of acetic anhydride was cooled in an ice bath. To this was added dropwise 2 mL of formic acid and the mixture was heated to 50° C. for 15 min. A 2 mL sample of the resulting solution was added dropwise to a cold mixture of 4-[(8-aza-bicyclo[3.2.1]oct-3-ylidene)-pyridin-3-yl-methyl]-N-ethyl-benzamide (1 g, 0.0029 mol) in 3 mL of THF. The resulting mixture was heated to 50° C. for 30 min, then diluted with CH₂Cl₂ and washed carefully with NaHCO₃ (aq), then brine, and dried (Na₂SO₄). The solvent was removed in vacuo and the residue was chromatographed on silica gel (9:1 CH₂Cl₂:MeOH) to give 104 mg of N-ethyl-4-[(8-formyl-8-aza-bicyclo[3.2.1]oct-3-ylidene)-pyridin-3-yl-methyl]-benzamide. MS m/z (MH⁺) 376.9; ¹H NMR 300 MHz (CDCl₃) δ 8.2 (d, 2H); 7.8 (d, 4H); 7.3 (d, 4H); 3.5 (q, 2H); 3.3-3.0 (m, 2H); 2.6-2.1 (m, 4H); 2.0-1.6 (m, 3H); 1.1 (t, 3H).

### EXAMPLE 91

### N-Ethyl-4-[(8-phenylacetyl-8-aza-bicyclo[3.2.1]oct-3-ylidene)-pyridin-3-yl-methyl]-benzamide (Cpd 200)

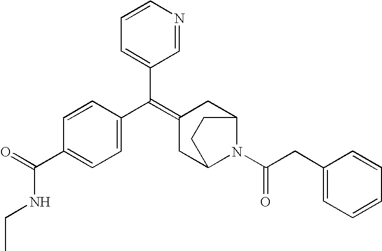

A 2.5 g (0.72 mmol) sample of 4-[(8-aza-bicyclo[3.2.1]oct-3-ylidene)-pyridin-3-yl-methyl]-N-ethyl-benzamide, 0.08 mL (1.4 mmol) of phenylacetyl chloride, and 0.1 g (2.16 mmol) of K₂CO₃ in 5 mL of DMF was refluxed for 1 h, then stirred overnight at room temperature. Water was added and the reaction mixture was extracted with an Et₂O/THF mixture. The organic phase was washed with brine and dried (Na₂SO₄). The solvent was removed in vacuo and the residue chromatographed on silica gel (9:1 CH₂Cl₂:MeOH). The resulting product was treated with Et₂O/HCl to give 0.02 g of N-ethyl-4-[(8-phenylacetyl-8-aza-bicyclo[3.2.1]oct-3-ylidene)-pyridin-3-yl-methyl]-benzamide. MS m/z (MH⁺) 466.3; ¹H NMR 300 MHz (DMSO-d₆) δ 8.7 (m, 2H); 8.2 (m, 2H); 7.9 (m, 4H); 7.3 (m, 7H); 4.5 (d, 2H); 3.3 (m, 2H); 2.4-2.1(m, 4H); 1.9-1.5 (m, 6H); 1.1 (q, 3H).

### Procedure Y

### 4-(8-Phenethyl-8-aza-bicyclo[3.2.1]oct-3-ylidenemethyl)-benzoic acid methyl ester-4-(8-Phenethyl-8-aza-bicyclo[3.2.1]oct-3-ylidenemethyl)-benzoic acid methyl ester

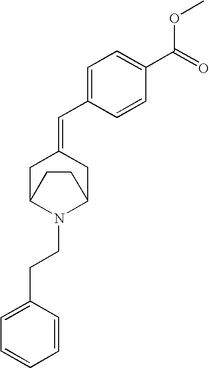

A 1.3 g (0.039 mol) sample of 60% NaH in oil was washed with methylcyclohexane and 10 mL of DMF was added. A solution of N-phenethyltropinone (7.0 g, 0.03 mol) and 4-(dimethoxyphosphoryl-methyl)-benzoic acid methyl ester (8.5 g, 0.033 mol) in 100 mL of DMF was added rapidly and the reaction was refluxed overnight. After cooling the reaction, the mixture was partitioned between Et₂O and water. The organic phase was washed sequentially with water and brine, then dried (Na₂SO₄). The solvent was evaporated in vacuo to give 10.54 g of 4-(dimethoxy-phosphorylmethyl)-benzoic acid methyl ester. MS m/z (MH⁺) 362.

### EXAMPLE 92

### N-Ethyl-4-(8-phenethyl-8-aza-bicyclo[3.2.1]oct-3-ylidenemethyl)-benzamide (Cpd 50)

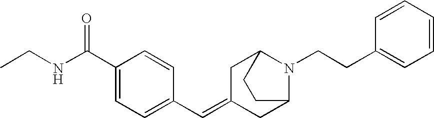

Using the procedures described in Example 71, 4-(dimethoxy-phosphorylmethyl)-benzoic acid methyl ester was converted to N-ethyl-4-(8-phenethyl-8-aza-bicyclo[3.2.1]oct-3-ylidenemethyl)-benzamide. MS m/z (MH⁺) 362.1.

### EXAMPLE 93

### (+)—N-Ethyl-4-(8-phenethyl-8-aza-bicyclo[3.2.1]oct-3-ylidenemethyl)-benzamide (Cpd 41) and

### (−)—N-Ethyl-4-(8-phenethyl-8-aza-bicyclo[3.2.1]oct-3-ylidenemethyl)-benzamide (Cpd 42)

N-ethyl-4-(8-phenethyl-8-aza-bicyclo[3.2.1]oct-3-ylidenemethyl)-benzamide was chromatographed on a CHIRALCEL® AS™ eluting with 1:1 MeOH:EtOH. The first enantiomer to elute was converted to its HCl salt with Et₂O/HCl in EtOH. [α]_{D}²⁵=+135°. MS m/z (MH⁺) 375.

The second enantiomer to elute in the chromatography from the foregoing example was collected. [α]D²⁵=−127°. MS m/z (MH⁺) 375.

### Procedure Z

### 4-[(1R,5S)-(8-Aza-bicyclo[3.2.1]oct-3-ylidene)-bromo-methyl]-N-ethyl-benzamide (Cpd 92)

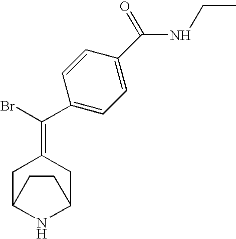

A 3.4 g (8.1 mmol) sample of 3-[bromo-(4-ethylcarbamoyl-phenyl)-methylene]-(1R,5S)-8-aza-bicyclo[3.2.1]octane-8-carboxylic acid ethyl ester was placed in a pressure tube with 100 mL of CHCl₃ and 2.0 mL of TMSI. The reaction was heated on a steambath for 4 h., cooled and 15 mL of MeOH were added. After stirring for 30 min, the reaction was partitioned between CHCl₃ and 3 N NaOH. The organic phase was washed with brine and then dried (Na₂SO₄). The solvent was evaporated in vacuo and the residue chromatographed on silica gel (80:20:2 CH₂Cl₂:MeOH:NH₄OH) to give 1.1 g of 4-[(1R,5S)-(8-aza-bicyclo[3.2.1]oct-3-ylidene)-bromo-methyl]-N-ethyl-benzamide. MS m/z (MH⁺) 349.1.

### Procedure AA

### 4-[(1S,5R)-(8-Aza-bicyclo[3.2.1]oct-3-ylidene)-bromo-methyl]-N-ethyl-benzamide

Following the protocol of Procedure 7 and substituting 3-[bromo-(4-ethylcarbamoyl-phenyl)-methylene]-(1S,5R)-8-aza-bicyclo[3.2.1]octane-8-carboxylic acid ethyl ester for 3-[bromo-(4-ethylcarbamoyl-phenyl)-methylene]-(1R,5S)-8-aza-bicyclo[3.2.1]octane-8-carboxylic acid ethyl ester the title compound was prepared. MS m/z (MH⁺) 349.1

### Procedure AB

### 4-[Bromo-(8-phenylacetyl-8-(1R,5S)-aza-bicyclo[3.2.1]oct-3-ylidene)-methyl]-N-ethyl-benzamide

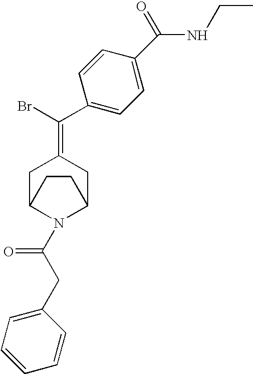

Using the protocol described in Example W, substituting 4-[(1R,5S)-(8-aza-bicyclo[3.2.1]oct-3-ylidene)-bromo-methyl]-N-ethyl-benzamide for 4-[(8-aza-bicyclo[3.2.1]oct-3-ylidene)-pyridin-3-yl-methyl]-N-ethyl-benzamide, the title compound was prepared. MS m/z (MH⁺) 468.

### Procedure AC

### 4-[Bromo-(8-phenylacetyl-8-(1S,5R)-aza-bicyclo[3.2.1]oct-3-ylidene)-methyl]-N-ethyl-benzamide

Using the protocol described in Example 91, substituting 4-[(1S,5R)-(8-aza-bicyclo[3.2.1]oct-3-ylidene)-bromo-methyl]-N-ethyl-benzamide for 4-[(8-aza-bicyclo[3.2.1]oct-3-ylidene)-pyridin-3-yl-methyl]-N-ethyl-benzamide, the title compound was prepared. MS m/z (MH⁺) 468.

### EXAMPLE 94

### N-Ethyl-4-[((1R,5S)-8-phenylacetyl-8-aza-bicyclo[3.2.1]oct-3-ylidene)-pyridin-3-yl-methyl]-benzamide (Cpd 217) and

### N-Ethyl-4-[((1S,5R)-8-phenylacetyl-8-aza-bicyclo[3.2.1]oct-3-ylidene)-pyridin-3-yl-methyl]-benzamide (Cpd 218)

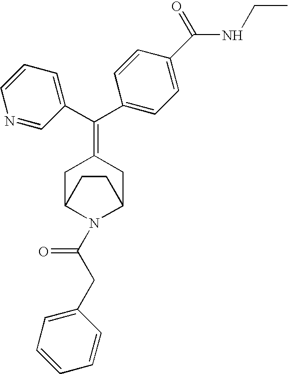

Using the protocol described in Example 75, substituting 4-[bromo-((1R,5S)-8-phenylacetyl-8-aza-bicyclo[3.2.1]oct-3-ylidene)-methyl]-N-ethyl-benzamide for 75a and 3-pyridylboronic acid for 75b, the title compound was prepared. MS m/z (MH⁺) 466.3.

### EXAMPLE 95

### 3-[(4-Ethylcarbamoyl-phenyl)-(4-hydroxy-3,5-dimethyl-phenyl)-methylene]-8-aza-bicyclo[3.2.1]octane-8-carboxylic acid ethyl ester (Cpd 173)

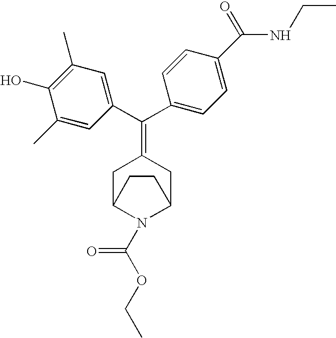

By the protocol of Example 75, substituting 3-[bromo-(4-ethylcarbamoyl-phenyl)-methylene]-8-aza-bicyclo[3.2.1]octane-8-carboxylic acid ethyl ester for 75a and 4-hydroxy-3,5-dimethylphenylboronic acid for 75b, the title compound was prepared. MS m/z (MH⁺) 463.2

### EXAMPLE 96

### 3-[(4-Ethylcarbamoyl-phenyl)-(4-hydroxy-3-methoxy-phenyl)-methylene]-8-aza-bicyclo[3.2.1]octane-8-carboxylic acid ethyl ester (Cpd 173)

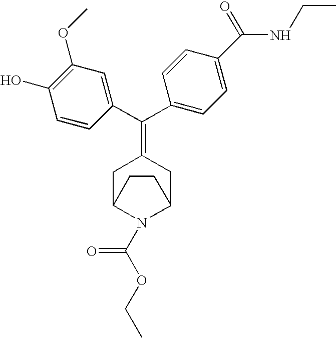

By the protocol of Example 75, substituting 3-[bromo-(4-ethylcarbamoyl-phenyl)-methylene]-8-aza-bicyclo[3.2.1]octane-8-carboxylic acid ethyl ester for 75a and 4-hydroxy-3-methoxyphenylboronic acid for 75b, the title compound was prepared. MS m/z (MH⁺) 465.1

### Procedure AD

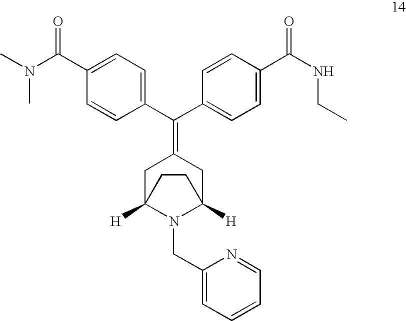

To 207 mg (0.59 mmol) of (+)-4-[(8-Aza-bicyclo[3.2.1]oct-3-ylidene)-bromo-methyl]-N-ethyl-benzamide was added DCE (5 mL), 2-pyridinecarboxaldehyde (94 mg, 0.88 mmol), acetic acid (50 μL), and sodium triacetoxyborohydride (186 mg, 0.88 mmol). The mixture was stirred at room temperature overnight. After quenching with water, the reaction was concentrated in vacuo. The crude residue was purified by colum chromatography (0-5% MeOH/CH₂Cl₂) to provide (+)-4-[Bromo-(8-pyridin-2-ylmethyl-8-aza-bicyclo[3.2.1]oct-3-ylidene)-methyl]-N-ethyl-benzamide (182 mg, 0.41 mmol).

### EXAMPLE 97

### N-Ethyl-4-[N′,N′-dimethyl benzamide-(8-pyridin-2-ylmethyl-8-aza-bicyclo[3.2.1]oct-3-ylidene)-methyl]-benzamide (Cpd 113)

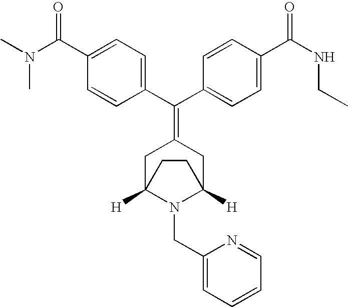

To a solution of 182 mg (0.41 mmol) (+)-4-[Bromo-(8-pyridin-2-ylmethyl-8-aza-bicyclo[3.2.1]oct-3-ylidene)-methyl]-N-ethyl-benzamide in N-methyl pyrrolidinone (2 mL) was added 170 mg (1.23 mmol) potassium carbonate in water (300 μL), 237 mg (1.23 mmol) 4-(dimethylaminocarbonyl) phenylboronic acid, and 23 mg (0.02 mmol) tetrakis(triphenylphosphine) palladium. The reaction was stirred at 85° C. for 18 h. After quenching with water, the mixture was absorbed onto diatomaceous earth and eluted with 5% MeOH/EtOAc. The eluate was concentrated to a residue and purified by reverse-phase chromatography. The free base was taken up in CH₂Cl₂ and precipitated with the addition of ethereal HCl to furnish (+)—N-Ethyl-4-[N′,N′-dimethylbenzamide-(8-pyridin-2-ylmethyl-8-aza-bicyclo[3.2.1]oct-3-ylidene)-methyl]-benzamide (114 mg, 0.20 mmol) as an HCl salt: MS m/z (MH⁺) 509.5; [α]_{D}²⁵=+2.51°.

### EXAMPLE 98

### N-Ethyl-4-[(4-hydroxymethyl-phenyl)-(8-thiophen-3-ylmethyl-8-aza-bicyclo[3.2.1]oct-3-ylidene)-methyl]-benzamide (Cpd 196)

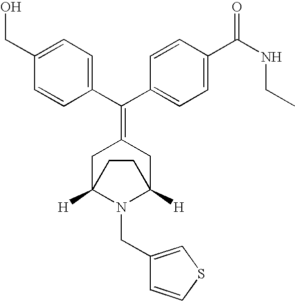

Following Procedure AD and substituting 3-thiophenecarboxaldehyde (96 mg, 0.86 mmol) for 2-pyridinecarboxaldehyde, (+)-4-[(8-Aza-bicyclo[3.2.1]oct-3-ylidene)-bromo-methyl]-N-ethyl-benzamide (200 mg, 0.57 mmol) was converted to (+)-4-[Bromo-(8-thiophen-3-ylmethyl-8-aza-bicyclo[3.2.1]oct-3-ylidene)-methyl]-N-ethyl-benzamide (179 mg, 0.40 mmol).

Following the procedure provided in Example 98, substituting 178 mg (0.40 mmol) (+)-4-[Bromo-(8-thiophen-3-ylmethyl-8-aza-bicyclo[3.2.1]oct-3-ylidene)-methyl]-N-ethyl-benzamide for (+)-4-[Bromo-(8-pyridin-2-yl methyl-8-aza-bicyclo[3.2.1]oct-3-ylidene)-methyl]-N-ethyl-benzamide, and 182 mg (1.20 mmol) 4-(hydroxymethyl)phenylboronic acid for 4-(dimethylaminocarbonyl)phenylboronic acid, the title Compound 196 (155 mg, 0.30 mmol) was obtained as an HCl salt: MS m/z (MH⁺) 473.3; [α]_{D}²⁵=+10.57°.

### EXAMPLE 99

### 4-(2-{3-[(4-Ethylcarbamoylphenyl)-phenylmethylene]-8-azabicyclo[3.2.1]oct-8-yl}-ethyl)piperazine-1-carboxylic acid methyl ester

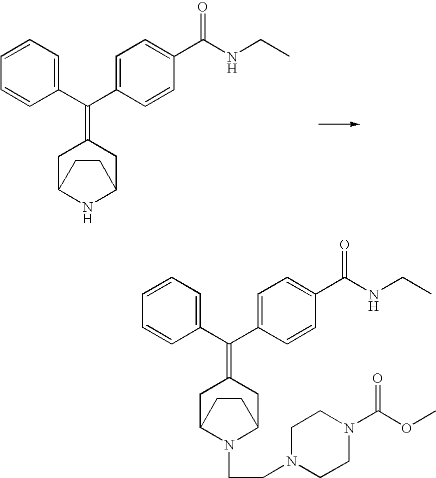

To a solution of 4-[(8-azabicyclo[3.2.1]oct-3ylidene)-phenylmethyl]-N-ethylbenzamide (0.5 g, 1.44 mmol) and 1-(2-chloroethyl)-4-carbomethoxypiperazine (0.3 g, 1.44 mmol) in 7 mL of acetonitrile was added 0.39 g (2.88 mmol) of potassium carbonate. The mixture was refluxed for 16 h. The solid was filtered and the solvent evaporated. The residue was partitioned between 1 N NaOH and CH₂Cl₂. The organic phase was concentrated and the residue was purified by flash chromatography (silica gel, CH₂Cl₂: 0.5 N NH₃ in MeOH, 9:1) to obtain 0.17 g of the title compound as a gum: MS m/z (M+1) 517.2.

### EXAMPLE 100

### 4-[Bromo-(8-phenethyl-8-azabicyclo[3.2.1]oct-3-ylidene)-methyl]-N,N-diethylbenzamide (Cpd 89)

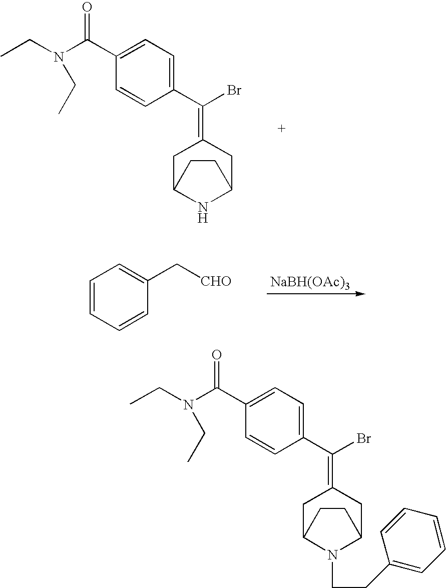

Using the procedure of Example 18 and substituting Compound 49 of Example 76 for N,N-diethyl-4-[(8-azabicyclo[3.2.1]oct-3-ylidene)phenylmethyl]benzamide and phenyl acetaldehyde for propionaldehyde, the title compound was obtained as a gum: Ms m/z (M+1) 481.2, 483.2.

### EXAMPLE 101

### 4-[(8-cyano-8-aza-bicyclo[3.2.1]oct-3-ylidene)-phenyl-methyl]-N,N-diethylbenzamide (Cpd 197)

Compound 197 was made using the literature method described in Knölker, Tetrahedron (1994) 50(37). 10893-10908.

Triethylamine was added dropwise to a stirred mixture of N,N-Diethyl-4-[(8-azabicyclo[3.2.1]oct-3-ylidene)phenylmethyl]benzamide (0.150 g, 0.268 mmol, Example 2), cyanogen bromide (0.213 g, 2.00 mmol), and DMAP in CH₂Cl₂ (0.5 mL). The mixture was stirred at room temperature for 4 h. At that time, water was cautiously added to the reaction, and the mixture was extracted with CH₂Cl₂. The organic phase was washed sequentially with water and brine, then dried (MgSO₄) and filtered. The filtrate was concentrated under reduced pressure at room temperature, and the resulting residue was purified by column chromatography (silica gel, 0 to 0.5% MeOH/CH₂Cl₂) to yield Compound 197 (0.110 g) as a white foam. MS m/z (MH⁺) 400.2.

### EXAMPLE 102

### 4-[(8-Carbamimidoyl-8-aza-bicyclo[3.2.1]oct-3-ylidene)-phenyl-methyl]-N,N-diethyl-benzamide (Cpd 198)

Preparation of Aluminum Reagent A

Ammonium chloride (0.045 g, 0.850 mmol) was suspended in 0.68 mL of toluene under an argon atmosphere at 0° C., and 0.42 mL of AlMe₃ (2.0 M in toluene, 0.850 mmol) was added dropwise. The mixture was stirred until gas evolution had ceased, and the reagent was used without further purification.

Compound 197 of Example 101 (0.170 g, 0.425 mmol) in 0.3 mL of toluene was added to a 1.25 M solution of Aluminum Reagent A. The mixture was heated to 80° C. for 24 h. The reaction was cooled to room temperature and concentrated under reduced pressure. The resulting residue was purified by reverse phase HPLC (25-70% CH₃CN/H₂O with 0.1% TFA in both solvents) to yield the title compound (3.9 mg) as the TFA salt. MS m/z (MH⁺) 417.1

### BIOLOGICAL EXAMPLES

Screening Assay for δ-Opioid and μ-Opioid Receptor Binding Rat Brain δ-Opioid Receptor Binding Assay

The activity of the compounds of the invention as analgesics was demonstrated by the rat brain δ-opioid receptor binding assay as described below.

Procedure

Male, Wistar rats (150-250 g, VAF, Charles River, Kingston, N.Y.) are killed by cervical dislocation, and their brains removed and placed immediately in ice cold Tris HCl buffer (50 mM, pH 7.4). The forebrains are separated from the remainder of the brain by a coronal transection, beginning dorsally at the colliculi and passing ventrally through the midbrain-pontine junction. After dissection, the forebrains are homogenized in Tris buffer in a Teflon®-glass homogenizer. The homogenate is diluted to a concentration of 1 g of forebrain tissue per 100 mL Tris buffer and centrifuged at 39,000×G for 10 min. The pellet is resuspended in the same volume of Tris buffer with several brief pulses from a Polytron homogenizer. This particulate preparation is used for the δ-opioid binding assays. Following incubation with the 6-selective peptide ligand [³H]DPDPE at 25° C., the tube contents are filtered through Whatman GF/B filter sheets on a Brandel cell harvester. The tubes

and filters are rinsed three times with 4 mL of 10 mM HEPES (pH 7.4), and the radioactivity associated with the filter circles determined using Formula 989 scintillation fluid (New England Nuclear, Boston, Mass.) in a scintillation counter.

Analysis

The data are used to calculate either the % inhibition compared to control binding (when only a single concentration of test compound is evaluated) or a Kᵢ value (when a range of concentrations is tested).

% Inhibition was calculated as follows:

$$1 - ( \frac{\text{(test compound dpm} - \text{nonspecific dpm)}}{\text{(total dpm} - \text{nonspecific dpm)}} ) \times 100 \%$$

Kᵢ value is calculated using the LIGAND (Munson, P. J. and Rodbard, D., Anal. Biochem. 107: 220-239, 1980) data analysis program.

Rat Brain μ-Opioid Receptor Binding Assay

The activity of compounds of the invention as analgesics is demonstrated by the rat brain 1-opioid receptor binding assay as described below.

Procedure

Male, Wistar rats (150-250 g, VAF, Charles River, Kingston, N.Y.) are killed by cervical dislocation and their brains removed and placed immediately in ice cold Tris HCl buffer (50 mM, pH 7.4). The forebrains are separated from the remainder of the brain by a coronal transection, beginning dorsally at the colliculi and passing ventrally through the midbrain-pontine junction. After dissection, the forebrains are homogenized in Tris buffer in a Teflon®-glass homogenizer. The homogenate is diluted to a concentration of 1 g of forebrain tissue per 100 mL Tris buffer and centrifuged at 39,000×G for 10 min. The pellet is resuspended in the same volume of Tris buffer with several brief pulses from a Polytron homogenizer. This particulate preparation is used for the μ-opioid binding assays. Following incubation with the m-selective peptide ligand [³H]DAMGO at 25° C., the tube contents are filtered through Whatman GF/B filter sheets on a Brandel cell harvester. The tubes and filters are rinsed three times with 4 mL of 10 mM HEPES (pH 7.4) and the radioactivity associated with the filter circles determined using Formula 989 scintillation fluid (New England Nuclear, Boston, Mass.) in a scintillation counter.

Analysis

The data are used to calculate either the % inhibition compared to control binding (when only a single concentration of test compound is evaluated) or a Kᵢ value (when a range of concentrations is tested).

% Inhibition is calculated as follows:

$$1 - ( \frac{\text{(test compound dpm} - \text{nonspecific dpm)}}{\text{(total dpm} - \text{nonspecific dpm)}} ) \times 100 \%$$

Kᵢ value is calculated using the LIGAND (Munson, P. J. and Rodbard, D., Anal. Biochem. 107: 220-239, 1980) data analysis program.

Table 2 shows the biological activity (in Kᵢ value) for 10 nM solutions of the present compounds as measured in the rat brain δ and μ opioid receptor binding assay.

TABLE 2

| | DOR binding | MOR binding |
|---|---|---|
| Cpd | Ki (nM)_{—} | Ki (nM)_{—} |
| 1 | 406.85 | 340.1 |
| 2 | 188.75 | 68.83 |
| 3 | 9.3 | 74.09 |
| 4 | 30.89 | 107.06 |
| 5 | 49.25 | 72.95 |
| 6 | 9.96 | 35.85 |
| 7 | 1.3 | 3.05 |
| 8 | 2.26 | 7.41 |
| 9 | 13.87 | 34.06 |
| 10 | 1.08 | 1.06 |
| 11 | 33.95 | 46.2 |
| 12 | 14.31 | 128.45 |
| 13 | 18.43 | 123.35 |
| 14 | 400.7 | 959.4 |
| 15 | 11.42 | 126.8 |
| 16 | 4.69 | 82.04 |
| 17 | 4.04 | 20.32 |
| 18 | 19.09 | 561.15 |
| 19 | 3.89 | 34.44 |
| 20 | 4.73 | 129.25 |
| 21 | 109.21 | 48.23 |
| 22 | 48.27 | 58.54 |
| 23 | 6.92 | 6.23 |
| 24 | 22.43 | 21.7 |
| 25 | 99.24 | 95.02 |
| 26 | 0.77 | 1.86 |
| 27 | 134.46 | 99.83 |
| 28 | 72.84 | 69.64 |
| 29 | 44.93 | 12.46 |
| 30 | 20.38 | 15.02 |
| 31 | 59.16 | 25.6 |
| 32 | 249.95 | 385.2 |
| 33 | 238.33 | 197.56 |
| 34 | 281.05 | 1416.45 |
| 35 | 5175.2 | 532.12 |
| 36 | 92.82 | 109.4 |
| 37 | 5491.75 | 1470.35 |
| 38 | 5102.85 | 235.85 |
| 39 | 90.16 | 83.68 |
| 40 | 14.43 | 8.97 |
| 41 | 163.25 | 123.7 |
| 42 | 100000 | 5.99 |
| 43 | 86.29 | 721.25 |
| 44 | 2.2 | 58.34 |
| 45 | 29.78 | 23.67 |
| 46 | 6518.775 | 5480.075 |
| 47 | 12.58 | 124.8 |
| 48 | 4042 | 25765 |
| 49 | 23.75 | 100000 |
| 50 | 412.5 | 44.31 |
| 51 | 1410 | 865.65 |
| 52 | 150.6 | 6490 |
| 53 | 294.75 | 261.95 |
| 54 | 10000 | 130 |
| 55 | 1056.2 | 195.3 |
| 56 | 10155 | 1503.05 |
| 57 | 10000 | 1414 |
| 58 | 10000 | 1180.3 |
| 59 | 10000 | 31.63 |
| 60 | 1210.15 | 2088.5 |
| 61 | 81.45 | 71.24 |
| 62 | 148.455 | 8.56 |
| 63 | 2.41 | 47.81 |
| 64 | 1.43 | 30.78 |
| 65 | 2.56 | 42.59 |
| 66 | 1.97 | 141.9 |
| 67 | 77.21 | 118.5 |
| 68 | 34.41 | 155 |
| 69 | 77.53 | 133.2 |
| 70 | 22.32 | 41.09 |
| 71 | 5.14 | 14.63 |
| 72 | 6.83 | 11.11 |
| 73 | 6.54 | 35.94 |
| 74 | 10.62 | 37.48 |
| 75 | 5.92 | 22.72 |
| 76 | 35.32 | 184.49 |
| 77 | 2051 | 355.7 |
| 78 | 5596.5 | 270.85 |
| 79 | | |
| 80 | 4.06 | 118.53 |
| 81 | 3.5 | 27.68 |
| 82 | 4.36 | 374.7 |
| 83 | 624.65 | 17.17 |
| 84 | 325.1 | 5661 |
| 85 | 2.47 | 536.55 |
| 86 | 504.25 | 19.26 |
| 87 | 253.6 | 815.2 |
| 88 | 36.37 | 550.75 |
| 89 | 311 | 1008.2 |
| 90 | 102.39 | 405.9 |
| 91 | 212.3 | 175.3 |
| 92 | 167.7 | 114.6 |
| 93 | 480.1 | 279.6 |
| 94 | 480.2 | 322 |
| 95 | 144.3 | 421.3 |
| 96 | 6.62 | 63.55 |
| 97 | 5.33 | 11.85 |
| 98 | 6 | 203.1 |
| 99 | 4.82 | 5294.2 |
| 100 | 3.65 | 146.6 |
| 101 | 13.65 | 112.69 |
| 102 | 5.65 | 1317.45 |
| 103 | 5852.5 | 10610 |
| 104 | 2645.5 | 5614.5 |
| 105 | 4516.5 | 5617.5 |
| 106 | 6560.25 | 53.65 |
| 107 | 1571.55 | 22.76 |
| 108 | 104.87 | 10000 |
| 109 | 4.46 | 56.86 |
| 110 | 56.27 | 9140 |
| 111 | 4.57 | 6.7 |
| 112 | 1440.1 | 10000 |
| 113 | 1.33 | 36.65 |
| 114 | 56.44 | 4828.55 |
| 115 | 1.98 | 11.03 |
| 116 | 50.59 | 1506.5 |
| 117 | 0.96 | 35.29 |
| 118 | 57.37 | 10000 |
| 119 | 0.19 | 254 |
| 120 | 2.2 | 5361.6 |
| 121 | 0.45 | 6.69 |
| 122 | 2.62 | 70.42 |
| 123 | 0.64 | 34.22 |
| 124 | 9.29 | 94.64 |
| 125 | 1.44 | 47.62 |
| 126 | 0.23 | 1.45 |
| 127 | 0.78 | 2.96 |
| 128 | 0.28 | 4.27 |
| 129 | 0.34 | 6.73 |
| 130 | 11.29 | 221.05 |
| 131 | 11.24 | 151.65 |
| 132 | 0.93 | 163.8 |
| 133 | 142.15 | 580.6 |
| 134 | 10.53 | 7.57 |
| 135 | 1 | 318.1 |
| 136 | 0.2 | 3.03 |
| 137 | 1.52 | 9.74 |
| 138 | 15.86 | 49.52 |
| 139 | 0.33 | 1.54 |
| 140 | 9.38 | 217.3 |
| 141 | 5.00E−02 | 1.05 |
| 142 | 1.2 | 4.01 |
| 143 | 0.3 | 1.03 |
| 144 | 82.46 | 212.4 |
| 145 | 940.85 | 945 |
| 146 | 3.5 | 15.16 |
| 147 | 58.39 | 108.55 |
| 148 | 135.85 | 329.45 |
| 149 | 191.91 | 63.69 |
| 150 | 349.1 | 73.77 |
| 151 | 260.45 | 1.76 |
| 152 | 0.94 | 12.22 |
| 153 | 19.58 | 71.09 |
| 154 | 34.32 | 91.35 |
| 155 | 3.05 | 74.3 |
| 156 | 35.63 | 5454.05 |
| 157 | 13.17 | 8.57 |
| 158 | 163.2 | 11.98 |
| 159 | 77.4 | 13.46 |
| 160 | 86.15 | 7228.5 |
| 161 | 1.26 | 10000 |
| 162 | 7.23 | 848.85 |
| 163 | 0.99 | 49.33 |
| 164 | 1.23 | 2.32 |
| 165 | 1.62 | 54.12 |
| 166 | 23.19 | 50.32 |
| 167 | 8.91 | 5.35 |
| 168 | 0.18 | 394.95 |
| 169 | 0.74 | 14.25 |
| 170 | 0.73 | 1482.5 |
| 171 | | |
| 172 | 5148.9 | 712.75 |
| 173 | 932.4 | 2066 |
| 174 | 2.34 | 303.65 |
| 175 | 1.31 | 34.35 |
| 176 | 1.08 | 10.65 |
| 177 | 3.91 | 1.34 |
| 178 | 0.15 | 0.81 |
| 179 | 1.53 | 8.43 |
| 180 | 0.98 | 10.04 |
| 181 | 1.74 | 6.04 |
| 182 | 0.52 | 2.71 |
| 183 | 36.89 | 5053.4 |
| 184 | 4.95 | 0.66 |
| 185 | 30.24 | 58.88 |
| 186 | 3.9 | 28.6 |
| 187 | 1.44 | 121.14 |
| 188 | 53.895 | 490.6 |
| 189 | 14.199 | 13.675 |
| 190 | 0.2188 | 0.71695 |
| 191 | 11.9955 | 22.14 |
| 192 | 2.74 | 18.61 |
| 193 | 3.22 | 25.34 |
| 194 | 1.38 | 3.67 |
| 195 | 7.51 | 64.23 |
| 196 | 0.56 | 4.1 |
| 197 | 5272.5 | 10000 |
| 198 | 5.75 | 10000 |
| 199 | 135.75 | 5339.85 |
| 200 | 5229.75 | 13.29 |
| 201 | 10000 | 12300 |
| 202 | 10000 | 8123.5 |
| 203 | 284.425 | 5449.75 |
| 204 | 288.045 | 25340.65 |
| 205 | 2.04 | 17.21 |
| 206 | 16.83 | 80.1 |
| 207 | 10000 | 10000 |
| 208 | 1327.05 | 61.74 |
| 209 | 0.94 | 2.59 |
| 210 | 1.1 | 2.3 |
| 211 | 0.27 | 0.23 |
| 212 | 28.37 | 38.485 |
| 213 | 10000 | 10000 |
| 214 | 10000 | 580.45 |
| 215 | 30.75 | 3120.05 |
| 216 | 0.68 | 12.065 |
| 217 | 10000 | 3.0655 |
| 218 | 10000 | 3.54 |
| 219 | | |
| 220 | | |
| 221 | | |
| 222 | | |
| 223 | | |
| 224 | | |
| 225 | | |
| 226 | | |

Mouse Acetylcholine Bromide-Induced Abdominal Constriction Assay

The activity of compounds of the invention as analgesics was further demonstrated by the mouse acetylcholine bromide-induced abdominal constriction assay as described below.

Procedure

The mouse acetylcholine-induced abdominal constriction assay (as described by Collier et al. in Brit. J. Pharmacol. Chem. Ther., 1968, 32:295-310 with minor modifications) was used to assess analgesic potency of the compounds of formula (I). The test drugs or appropriate vehicles were administered orally (p.o.) and 30 min later the animal received an intraperitoneal (i.p.) injection of 5.5 mg/kg acetylcholine bromide (Matheson, Coleman and Bell, East Rutherford, N.J.). The mice were then placed in groups of three into glass bell jars and observed for a ten min observation period for the occurrence of an abdominal constriction response (defined as a wave of constriction and elongation passing caudally along the abdominal wall, accompanied by a twisting of the trunk and followed by extension of the hind limbs). For compounds of the present invention, the percent inhibition of this response to a nociceptive stimulus (equated to % analgesia) was calculated as follows:

$$\% Inhibition of response ( i . e . , \% analgesia ) = \frac{\begin{matrix}( No . of control animal responses - \\ No . of drug \text{-} treated animal responses )\end{matrix}}{No . of control animals responding .} \times 100$$

As a result of the mouse acetylcholine bromide-induced abdominal constriction assay, the compound of Example 1 measured an 87% inhibition response at a dose of 150 μmole/Kg p.o.

1. A compound of Formula (Ia): 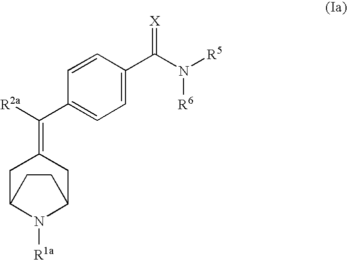
wherein:
R^{1a} is a substituent selected from the group consisting of hydrogen, C₁₋₆alkyl, —CH₂—(C₂₋₈alkenyl), cycloalkyl(C₁₋₄)alkyl, heterocyclyl(C₁₋₈)alkyl, aryl(C₁₋₈)alkyl, aryl(C₂₋₈)alkynyl, heteroaryl(C₁₋₈)alkyl, (R¹¹)₂—N—(C₁₋₈)alkyl, R¹—O—(C₁₋₈)alkyl-, R¹¹—S—(C₁₋₈)alkyl, R¹¹—SO—(C₁₋₈)alkyl, and R¹¹—SO₂—(C₁₋₈)alkyl; wherein heterocyclyl is optionally substituted with one to three substituents independently selected from the group consisting of C₁₋₆alkyl, C₁₋₆alkoxy, C₁₋₆alkoxycarbonyl, C₁₋₆alkylcarbonylamino, C₁₋₆alkylthio, C₁₋₆alkylsulfonyl, halogen, and oxo; and wherein aryl and heteroaryl are optionally substituted with one to three substituents independently selected from the group consisting of C₁₋₆alkyl, C₂₋₆alkenyl, C₁₋₆alkoxy, amino, C₁₋₆alkylamino, di(C₁₋₆alkyl)amino, C₁₋₆alkylcarbonyl, C₁₋₆alkoxycarbonylamino, C₁₋₆alkylthio, C₁₋₆alkylsulfonyl, heterocyclyl, cyano, halogen, hydroxy, trifluoromethyl and trifluoromethoxy; wherein R¹¹ is hydrogen, C₁₋₈alkyl or aryl;
R^{2a} is a substituent selected from hydrogen, halogen, cyano, [1,3]-benzodioxolyl, quinolinyl, tetrazolyl, or aryl; wherein aryl is substituted with one to three substituents independently selected from the group consisting of C₁₋₄alkyl, carboxy, amino and carboxy, nitro, di(C₁₋₆alkyl)aminocarbonyl, (C₁₋₆alkyl)aminocarbonyl, aminocarbonyl, aminosulfonyl, or tetrazolyl; and
wherein alkyl is substituted with one to three substituents selected from amino, hydroxy, or carboxy;
X is selected from O or S;
R⁵ and R⁶ are independently selected from hydrogen or C₁₋₈alkyl; and
pharmaceutically acceptable enantiomers, diastereomers and salts thereof.

2. A compound of Formula (Ib): 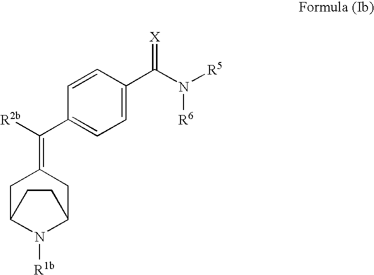
wherein:
R^{1b} is a substituent selected from the group consisting of (1-benzyl-1-amino)ethyl, 1-benzyl-1-(t-butoxycarbonylamino)ethyl, 2-(4-alkoxycarbonylpiperazin-1-yl)eth-1-yl, 3-dimethylaminocarbonyl-3,3-diphenylprop-1-yl, 3-cyano-3,3-diphenylprop-1-yl, tetrazolyl(C₁₋₃)alkyl, quinolinyl(C₁₋₃)alkyl, aryl(C₁₋₄)alkyl, aryl(C₁₋₄)alkylcarbonyl, heteroarylcarbonyl, (halo-arylcarbonyl)heteroarylcarbonyl(C₁₋₃)alkyl, (C₁₋₄)alkoxycarbonyl, cyano, cyano(C₁₋₃)alkyl, formyl, and aminoiminomethyl; wherein aryl and heteroaryl are substituted with one to three substituents independently selected from the group consisting of C₁₋₆alkylcarbonylamino, carboxy, and nitro;
R^{2b} is a substituent selected from aryl or heteroaryl; wherein aryl and monocyclic heteroaryl are optionally substituted with C₁₋₆alkyl, C₁₋₆alkoxy, amino, C₁₋₆alkylamino, di(C₁₋₆alkyl)amino, C₁₋₆alkylcarbonyl, C₁₋₆alkylcarbonylamino, C₁₋₆alkylthio, C₁₋₆alkylsulfonylamino, halogen, hydroxy, cyano, trifluoromethyl and trifluoromethoxy;
X is selected from O or S;
R⁵ and R⁶ are independently selected from hydrogen or C₁₋₈alkyl; and
pharmaceutically acceptable enantiomers, diastereomers and salts thereof.

3. A compound of Formula (Ic): 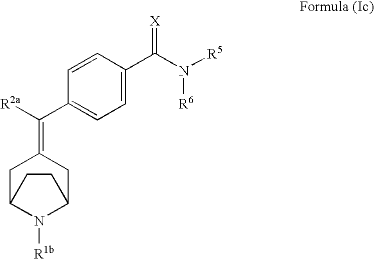
wherein:
R^{1b} is a substituent selected from the group consisting of (1-benzyl-1-amino)ethyl, 1-benzyl-1-(t-butoxycarbonylamino)ethyl, 2-(4-alkoxycarbonylpiperazin-1-yl)eth-1-yl, 3-dimethylaminocarbonyl-3,3-diphenylprop-1-yl, 3-cyano-3,3-diphenylprop-1-yl, tetrazolyl(C₁₋₃)alkyl, quinolinyl(C₁₋₃)alkyl, aryl(C₁₋₄)alkyl, aryl(C₁₋₄)alkylcarbonyl, heteroarylcarbonyl, (halo-arylcarbonyl)heteroarylcarbonyl(C₁₋₃)alkyl, (C₁₋₄)alkoxycarbonyl, cyano, cyano(C₁₋₃)alkyl, formyl, and aminoiminomethyl; wherein aryl and heteroaryl are substituted with one to three substituents independently selected from the group consisting of C₁₋₆alkylcarbonylamino, carboxy, and nitro;
R^{2a} is a substituent selected from hydrogen, halogen, cyano, [1,3]-benzodioxolyl, quinolinyl, tetrazolyl, or aryl; wherein aryl is substituted with one to three substituents independently selected from the group consisting of C₁₋₆alkyl, carboxy, amino and carboxy, nitro, di(C₁₋₆alkyl)aminocarbonyl, (C₁₋₆alkyl)aminocarbonyl, aminocarbonyl, aminosulfonyl, or tetrazolyl; and wherein alkyl is substituted with one to three substituents selected from amino, hydroxy, or carboxy;
X is selected from O or S;
R⁵ and R⁶ are independently selected from hydrogen or C₁₋₈alkyl; and
pharmaceutically acceptable enantiomers, diastereomers and salts thereof.

4. A compound according to claim 1 wherein R^{1a} is selected from the group consisting of hydrogen, —CH₂—C₂₋₆alkenyl, heterocyclyl(C₁₋₃)alkyl, heteroaryl(C₁₋₃)alkyl, aryl(C₁₋₃)alkyl, aryl(C₂₋₃)alkynyl; and wherein aryl and heteroaryl are independently and optionally substituted with one to three substituents independently selected from the group consisting of C₁₋₆alkyl, C₁₋₆alkoxy, C₁₋₆alkylcarbonylamino, halogen, hydroxy, C₁₋₆alkylcarbonyl, and cyano.

5. A compound according to claim 3 wherein R^{1a} is selected from the group consisting of hydrogen, —CH₂—C₂₋₆alkenyl, heterocyclyl(C₁₋₃)alkyl, heteroaryl(C₁₋₃)alkyl, aryl(C₁₋₃)alkyl, aryl(C₂₋₃)alkynyl; and wherein aryl and heteroaryl are independently and optionally substituted with one to three substituents independently selected from the group consisting of C₁₋₆alkyl, C₁₋₆alkoxy, C₁₋₆alkylcarbonylamino, halogen, hydroxy, C₁₋₆alkylcarbonyl, and cyano.

6. A compound according to claim 1 wherein R^{1a} is selected from the group consisting of hydrogen, 3,3-dimethallyl, (1,3)-benzodioxol-5-yl(C₁₋₃)alkyl, phenyl(C₁₋₃)alkyl, phenyl(C₂₋₃)alkynyl, imidazolinyl(C₁₋₃)alkyl, furyl(C₁₋₃)alkyl, thiophenyl(C₁₋₃)alkyl, thiazolyl(C₁₋₃)alkyl, imidazolyl(C₁₋₃)alkyl, and pyridinyl(C₁₋₃)alkyl; and wherein thiophenyl, furyl, imidazolyl, and phenyl are independenitly and optionally substituted with one to three substituents selected from halogen, C₁₋₃alkylcarbonylamino, and C₁₋₃alkyl.

7. A compound according to claim 3 wherein R^{1a} is selected from the group consisting of hydrogen, 3,3-dimethallyl, (1,3)-benzodioxol-5-yl(C₁₋₃)alkyl, phenyl(C₁₋₃)alkyl, phenyl(C₂₋₃)alkynyl, imidazolinyl(C₁₋₃)alkyl, furyl(C₁₋₃)alkyl, thiophenyl(C₁₋₃)alkyl, thiazolyl(C₁₋₃)alkyl, imidazolyl(C₁₋₃)alkyl, and pyridinyl(C₁₋₃)alkyl; and wherein thiophenyl, furyl, imidazolyl, and phenyl are independenitly and optionally substituted with one to three substituents selected from halogen, C₁₋₃alkylcarbonylamino, and C₁₋₃alkyl.

8. A compound according to claim 1 wherein, R¹¹ is independently selected from the group consisting of hydrogen, C₁₋₈alkyl and aryl.

9. A compound according to claim 3 wherein, R¹¹ is independently selected from the group consisting of hydrogen, C₁₋₈alkyl and aryl.

10. A compound according to claim 1 wherein R¹¹ is independently selected from the group consisting of hydrogen, methyl, and phenyl.

11. A compound according to claim 3 wherein R¹¹ is independently selected from the group consisting of hydrogen, methyl, and phenyl.

12. A compound according to claim 1 wherein R^{1a} is selected from the group consisting of hydrogen, 3,3-dimethallyl, phenethyl, phenylpropyl, imidazolyl methyl, thiophenyl methyl, (1,3)-benzodioxol-5-ylmethyl, pyridinylmethyl, thiazolylmethyl, and furylmethyl; wherein phenyl and thiophenyl are optionally substituted with one to two substituents selected from halogen, acetamido, or methyl.

13. A compound according to claim 3 wherein R^{1a} is selected from the group consisting of hydrogen, 3,3-dimethallyl, phenethyl, phenylpropyl, imidazolyl methyl, thiophenyl methyl, (1,3)-benzodioxol-5-ylmethyl, pyridinylmethyl, thiazolylmethyl, and furylmethyl; wherein phenyl and thiophenyl are optionally substituted with one to two substituents selected from halogen, acetamido, or methyl.

14. A compound according to claim 1 wherein R^{2a} is selected from the group consisting of hydrogen, halogen, cyano, phenyl, tetrazolyl, 1,3-benzodioxolyl, and quinolinyl; wherein phenyl is substituted with one to three substituents independently selected from the group consisting of C₁₋₃alkyl, amino (when said phenyl is also substituted with carboxy), aminocarbonyl, C₁₋₆alkylaminocarbonyl, di(C₁₋₆alkyl)aminocarbonyl, aminosulfonyl, heteroaryl, nitro, and carboxy; wherein alkyl is substituted with one to three substituents independently selected from amino, C₁₋₆alkylamino, di(C₁₋₆alkyl)amino, hydroxy, or carboxy.

15. A compound according to claim 3 wherein R^{2a} is selected from the group consisting of hydrogen, halogen, cyano, phenyl, tetrazolyl, 1,3-benzodioxolyl, and quinolinyl; wherein phenyl is substituted with one to three substituents independently selected from the group consisting of C₁₋₃alkyl, amino (when said phenyl is also substituted with carboxy), aminocarbonyl, C₁₋₆alkylaminocarbonyl, di(C₁₋₆alkyl)aminocarbonyl, aminosulfonyl, heteroaryl, nitro, and carboxy; wherein alkyl is substituted with one to three substituents independently selected from amino, C₁₋₆alkylamino, di(C₁₋₆alkyl)amino, hydroxy, or carboxy.

16. A compound according to claim 1 wherein R^{2a} is selected from the group consisting of hydrogen, halogen, cyano, phenyl, tetrazolyl, and (1,3)-benzodioxolyl; wherein phenyl is optionally substituted with one to three substituents independently selected from the group consisting of C₁₋₄alkyl, aminocarbonyl, alkylaminocarbonyl, di(C₁₋₆alkyl)aminocarbonyl, aminosulfonyl, heteroaryl, nitro, carboxy, and cyano; wherein tetrazolyl is optionally substituted with C₁₋₃alkyl; and wherein alkyl is substituted with one to three substituents independently selected from amino, hydroxy, and carboxy.

17. A compound according to claim 3 wherein R^{2a} is selected from the group consisting of hydrogen, halogen, cyano, phenyl, tetrazolyl, and (1,3)-benzodioxolyl; wherein phenyl is optionally substituted with one to three substituents independently selected from the group consisting of C₁₋₄alkyl, aminocarbonyl, alkylaminocarbonyl, di(C₁₋₆alkyl)aminocarbonyl, aminosulfonyl, heteroaryl, nitro, carboxy, and cyano; wherein tetrazolyl is optionally substituted with C₁₋₃alkyl; and wherein alkyl is substituted with one to three substituents independently selected from amino, hydroxy, and carboxy.

18. A compound according to claim 1 wherein R^{2a} is selected from the group consisting of hydrogen, bromine, cyano, phenyl, tetrazolyl, and (1,3)-benzodioxolyl; wherein phenyl is optionally substituted with one to three substituents independently selected from the group consisting of aminocarbonyl, ethylaminocarbonyl, dimethylaminocarbonyl, hydroxymethyl, carboxyethyl, carboxy(1-amino)ethyl, aminosulfonyl, tetrazolyl, nitro, and carboxy.

19. A compound according to claim 3 wherein R^{2a} is selected from the group consisting of hydrogen, bromine, cyano, phenyl, tetrazolyl, and (1,3)-benzodioxolyl; wherein phenyl is optionally substituted with one to three substituents independently selected from the group consisting of aminocarbonyl, ethylaminocarbonyl, dimethylaminocarbonyl, hydroxymethyl, carboxyethyl, carboxy(1-amino)ethyl, aminosulfonyl, tetrazolyl, nitro, and carboxy.

20. A compound according to claim 2 wherein R^{1b} is selected from the group consisting of aryl(C₁₋₄)alkylcarbonyl, heteroaryl(C₁₋₄)alkyl, heteroarylcarbonyl, cyano(C₁₋₄)alkyl, quinolinyl(C₁₋₃)alkyl, (3-dimethylaminocarbonyl-3,3-diphenylprop-1-yl, (1-benzyl-1-amino)ethyl, 2-(4-alkoxycarbonylpiperazin-1-yl)eth-1-yl, 3-cyano-3,3-diphenylprop-1-yl, (halo-arylcarbonyl)heteroarylcarbonyl(C₁₋₃)alkyl, tetrazolyl(C₁₋₃)alkyl, (C₁₋₄)alkoxycarbonyl, and aminoiminomethyl; wherein heteroaryl is substituted with one to three substituents independently selected from carboxy, halogen, or nitro.

21. A compound according to claim 3 wherein R^{1b} is selected from the group consisting of aryl(C₁₋₄)alkylcarbonyl, heteroaryl(C₁₋₄)alkyl, heteroarylcarbonyl, cyano(C₁₋₄)alkyl, quinolinyl(C₁₋₃)alkyl, (3-dimethylaminocarbonyl-3,3-diphenylprop-1-yl, (1-benzyl-1-amino)ethyl, 2-(4-alkoxycarbonylpiperazin-1-yl)eth-1-yl, 3-cyano-3,3-diphenylprop-1-yl, (halo-arylcarbonyl)heteroarylcarbonyl(C₁₋₃)alkyl, tetrazolyl(C₁₋₃)alkyl, (C₁₋₄)alkoxycarbonyl, and aminoiminomethyl; wherein heteroaryl is substituted with one to three substituents independently selected from carboxy, halogen, or nitro.

22. A compound according to claim 2 wherein R^{1b} is selected from the group consisting of quinolinyl(C₁₋₃)alkyl, aminoiminomethyl, aryl(C₁₋₄)alkylcarbonyl, and heteroaryl(C₁₋₄)alkyl wherein heteroaryl is substituted with nitro.

23. A compound according to claim 3 wherein R^{1b} is selected from the group consisting of quinolinyl(C₁₋₃)alkyl, aminoiminomethyl, aryl(C₁₋₄)alkylcarbonyl, and heteroaryl(C₁₋₄)alkyl wherein heteroaryl is substituted with nitro.

24. A compound according to claim 2 wherein R^{1b} is selected from thiophenylcarbonyl, 5-nitro-thiophen-3-yl, quinolin-2-ylmethyl, benzylcarbonyl, or aminoiminomethyl.

25. A compound according to claim 3 wherein R^{1b} is selected from thiophenylcarbonyl, 5-nitro-thiophen-3-yl, quinolin-2-ylmethyl, benzylcarbonyl, or aminoiminomethyl.

26. A compound according to claim 2 wherein R^{2b} is selected from aryl or heteroaryl; wherein aryl and heteroaryl are optionally substituted with C₁₋₆alkyl, amino, C₁₋₆alkylcarbonylamino, halogen, and cyano.

27. A compound according to claim 3 wherein R^{2b} is selected from aryl or heteroaryl; wherein aryl and heteroaryl are optionally substituted with C₁₋₆alkyl, amino, C₁₋₆alkylcarbonylamino, halogen, and cyano.

28. A compound according to claim 2 wherein R^{2b} is selected from aryl, pyridinyl, pyrimidinyl, or pyrazinyl; wherein aryl is optionally substituted with amino, C₁₋₆alkylcarbonyl, C₁₋₆alkylcarbonylamino, halogen, or cyano.

29. A compound according to claim 3 wherein R^{2b} is selected from aryl, pyridinyl, pyrimidinyl, or pyrazinyl; wherein aryl is optionally substituted with amino, C₁₋₆alkylcarbonyl, C₁₋₆alkylcarbonylamino, halogen, or cyano.

30. A compound according to claim 2 wherein R^{2b} is selected from phenyl or pyridinyl; wherein phenyl is optionally substituted with a substituent selected from amino, methylcarbonyl, methylcarbonylamino, fluorine, or cyano.

31. A compound according to claim 3 wherein R^{2b} is selected from phenyl or pyridinyl; wherein phenyl is optionally substituted with a substituent selected from amino, methylcarbonyl, methylcarbonylamino, fluorine, or cyano.

32. A compound according to claim 1 wherein X is O.

33. A compound according to claim 1 wherein R⁵ and R⁶ are independently selected from the group consisting of hydrogen and C₁₋₄alkyl.

34. A compound according to claim 1 wherein R⁵ and R⁶ are independently selected from the group consisting of hydrogen, methyl, and ethyl.
